(19) United States
(12) Patent Application Publication
(10) Pub. No.: US 2024/0285687 A1
(43) Pub. Date: Aug. 29, 2024

(54) NON-NATURAL NKG2D RECEPTORS THAT DO NOT DIRECTLY SIGNAL THE CELLS TO WHICH THEY ARE ATTACHED

(71) Applicant: XYPHOS BIOSCIENCES INC., South San Francisco, CA (US)

(72) Inventors: Kaman Kim, San Bruno, CA (US); David W. Martin, JR., Mill Valley, CA (US); Steven Williams, San Francisco, CA (US)

(21) Appl. No.: 18/606,822

(22) Filed: Mar. 15, 2024

Related U.S. Application Data
(63) Continuation of application No. 16/674,705, filed on Nov. 5, 2019, now Pat. No. 11,957,714.
(60) Provisional application No. 62/755,776, filed on Nov. 5, 2018.

(51) Int. Cl. A61K 35/17; A61K 47/64; C07K 14/705; C07K 19/00; C12N 5/07; C12N 5/078

(52) U.S. Cl. CPC A61K 35/17; A61K 47/6425; C07K 14/705; C07K 14/7056; C07K 14/70596; C07K 19/00; C12N 5/06; C12N 5/0634; C07K 2319/02; C07K 2319/03; C07K 2319/75

(57) ABSTRACT

The present disclosure relates to non-natural NKG2D receptors attached to mammalian cell surfaces wherein the non-natural receptors do not directly signal or directly activate the cell when the receptor is bound by cognate non-natural α1-α2 domains of NKG2D ligands modified to specifically bind the non-natural NKG2D receptors. The non-natural α1-α2 domains of NKG2D ligands may be attached to heterologous atoms or molecules including polypeptides, in some embodiments cytokines or modified cytokines, antibodies or fragments of antibodies.

### FIELD OF THE INVENTION

This application relates generally to a non-natural ectodomain of a non-natural NKG2D receptor attached to a mammalian cell wherein the receptor does not directly activate or directly signal the mammalian cell when bound by a non-natural NKG2D ligand modified to specifically bind the non-natural NKG2D receptor and to which heterologous molecules are attached to the modified α1-α2 domains of NKG2D ligand.

### CROSS REFERENCE TO RELATED APPLICATIONS

This application is a continuation of U.S. patent application Ser. No. 16/674,705, filed Nov. 5, 2019, which claims priority benefit of U.S. Provisional Patent Application No. 62/755,776, filed Nov. 5, 2018, each of which is incorporated herein in its entirety.

### INCORPORATION BY REFERENCE OF MATERIAL SUBMITTED ELECTRONICALLY

A Sequence Listing, which is a part of the present disclosure, is submitted concurrently with the specification in electronic format. The name of the file containing the Sequence Listing is “50004A_SeqListing.xml.” The Sequence Listing was created on Mar. 14, 2024, and is 387,589 bytes in size. The subject matter of the Sequence Listing is incorporated by reference herein in its entirety.

### BACKGROUND INFORMATION

NKG2D is an activating receptor expressed as a type II homodimeric integral protein on the surface of Natural Killer (NK) cells and certain T cells and macrophages. When bound to one of its eight natural ligands expressed primarily on the surfaces of distressed cells, the NKG2D activates the NK cell to kill the stressed cell, or when on T cells, the ligand-occupied NKG2D co-stimulates an activated T-cell to carry out its effector function. The three-dimensional structures have been solved for the ectodomain of human natural NKG2D, several of its soluble natural ligands and, in some cases, the bound complex of soluble ligand and receptor ectodomain. The monomeric α1-α2 domains of NKG2D ligands bind specifically to the two ectodomains of the natural NKG2D homodimer.

### SUMMARY OF THE INVENTION

The present disclosure relates to non-natural NKG2D receptors attached to mammalian cell surfaces wherein the non-natural receptors do not directly signal or directly activate the cell when the receptor is bound by cognate non-natural α1-α2 domains of NKG2D ligands modified to specifically bind the non-natural NKG2D receptors. The non-natural α1-α2 domains of NKG2D ligands may be attached to heterologous atoms or molecules including polypeptides, in some embodiments cytokines or modified cytokines, antibodies or fragments of antibodies. Direct activation of or direct signaling to the cell is not mediated by the attached non-natural NKG2D receptor and does not occur even when immunologic synapses have occurred.

### BRIEF DESCRIPTION OF THE DRAWINGS

FIGS. 1A-1B: (FIG. 1A) Alignment of the natural NKG2D.wt ectodomain (SEQ ID NO: 17) along with the NKG2D.YA (SEQ ID NO: 18) and NKG2D.AF (SEQ ID NO: 25) non-natural variants. Indicated are the locations of Y152 and Y199 and highlighted in gray are the mutated residues present in the non-natural variants. (FIG. 1B) Alignment of the α1-α2 domain of natural/wild-type ULBP2 (SEQ ID NO: 4) and non-natural variants of ULBP2 including ULBP2.R80W (SEQ ID NO: 108). Highlighted in gray are the residues critical for binding of non-natural ULBP2 variants to the non-natural NKG2D.YA or NKG2D.AF receptors. Indicated are the locations of residue R80 as well as the M154-F159 region that was explored for orthoganal variants binding to NKG2D.YA (ULBP2. S3, SEQ ID NO.: 127) or NKG2D.AF (ULBP2.C, SEQ ID NO.: 111; ULBP2.R, SEQ ID NO.: 113; ULPB2. AA, SEQ ID NO.: 115; and ULBP2. AB, SEQ ID NO.: 117).

FIG. 2: Size-exclusion chromatography comparison of non-natural Fc-NKG2D fusion proteins analyzed on an Akta HiLoad 16/600 Superdex 200 column. Migration of correctly assembled material was exemplified by a discrete, symmetrical peak that eluted at higher volumes while aggregated material eluted sooner at lower volumes. The site and nature of the modifications are indicated by the amino acid numbers Y152, Y199, or both (SEQ ID NOs: 48, 43, 42, 58, 41, and 40 starting from the top of the figure).

FIG. 3: Size-exclusion chromatography profiles of non-natural Fc-eNKG2D variants with one or two amino acid changes were analyzed on an Akta Superdex 200 Increase 10/300 GL column. Migration of correctly assembled material is exemplified by a discrete, symmetrical peak eluting at higher volumes while aggregated material—characterized by a low amplitude broad peak or series of peaks—eluted at lower volumes. The letters in parentheses represent the amino acids at positions 152 and 199 (SEQ ID NOs: 57, 56, 55, 54, 53, 52, 51, 50, 49, 48, 47, 46, 45, 44, and 40 in order from the top), respectively.

FIG. 4: ELISA binding of ULBP2 wild-type, MICwed- and MIC25-rituximab MicAbodies to Fc-eNKG2D candidates. Key is indicated at the top of the figure, but since many of the curves overlapped, individual curves were also labeled in each graph.

FIG. 5: Binding of eNKG2D variants to wild-type ligands. Wild-type ligands (all in Fc-fusion format) were captured onto Octet AHC biosensors, and each natural NKG2D, NKG2D.Y152A, or eNKG2D5 (Y152A/Y199F) as an Fc-fusion was titrated from 300 nM to 0.41 nM. Maximal binding responses were quantified by Octet. (Note the different ordinates for each graph.).

FIG. 6: Titration ELISA of individual phage variants to confirm selective binding to Fc-NKG2D.AF and reduced or eliminated binding to Fc-NKG2D.wt. Mutations are detailed in FIG. 22.

FIG. 7: ELISA data of four non-natural α1-α2 ULBP2 variant MicAbodies binding to NKG2D.wt, NKG2D.YA, and NKG2D.AF. The Fc-NKG2D variants were used as capture agents. MicAbodies were titrated in and detected with HRP-conjugated anti-human kappa.

FIGS. 8A-8B: ULBP2.C (SEQ ID NO: 111), ULBP2.R (SEQ ID NO: 113), ULBP2. AA (SEQ ID NO: 115), and ULBP2. AB (SEQ ID NO: 117) were examined for changes in peptide-MHCI immunogenicity relative to wild-type ULBP2 (SEQ ID NO: 4) using the NetMHC4.0 Server, querying against the HLA supertype representatives. For the input sequence, the variable region (residues 154-159 according to alignment in FIG. 1B) for each variant along with upstream and downstream nine residues (24 residues total) was entered and 9-mer peptide windows examined for predicted immunogenicity. Dark gray boxes correspond to peptides strongly predicted to bind the MHCI pocket (defined as having % rank<0.5) and therefore have a strong chance of being presented. Light gray boxes correspond to predicted weak binders (% rank<2). See Example 5 text for additional details.

FIGS. 9A-91: ULBP2.C (SEQ ID NO: 111), ULBP2.R (SEQ ID NO: 113), ULBP2. AA (SEQ ID NO: 115), and ULBP2. AB (SEQ ID NO: 117) were examined for changes in peptide-MHC class II immunogenicity relative to wild-type ULBP2 (SEQ ID NO: 4) using the NetMHCII 2.3 Server, querying against HLA-DR, HLA-DQ, HLA-DP. For the input sequence, the variable region (residues 154-159 according to alignment in FIG. 1B) for each variant along with upstream and downstream 15 residues (36 residues total) was entered and 15-mer peptide windows examined for predicted immunogenicity. Dark gray boxes correspond to peptides strongly predicted to bind the MHCII pocket and therefore likely to be presented and immunogenic. Light gray boxes correspond to predicted weak binders.

FIGS. 10A-10B: ELISA-measured binding of rituximab-MicAbodies comprised of ULBP2.wt (wild-type), ULBP2.R80W (which has enhanced affinity for wild-type NKG2D), ULPB2. S3 (NKG2D.YA-selected orthogonal variant), or ULBP2.R (NKG2D.AF-selected orthogonal variant) to natural NKG2D.wt, to NKG2D.YA, and to NKG2D.AF. (FIG. 10A) ELISA curves. The reduction in 458 nm absorption for some assays at higher concentrations is an artifact that is often seen with high affinity engagers at higher concentrations due to precipitation of the TMB-Ultra ELISA development reagent. (FIG. 10B) EC50 values (reported in nM) as determined in GraphPad Prism based upon the curves in (A). nd=not determined due to the lack of relationship between increased concentration and binding.

FIGS. 11A-11C: In vitro cytolytic assays with CD8 effector cells that were either untransduced or transduced with NKG2D.wt, NKG2D.YA, or NKG2D.AF CAR constructs consisting of the CD8a hinge/transmembrane domain and intracellular 4-1BB and CD3zeta signaling domains. Target cells were pre-loaded with calcein and exposed to effector cells at increasing effector to target (E:T) ratios. Released calcein was quantified after five hours. (FIG. 11A) Cell lysis of HeLa cells, (FIG. 11B) lysis of HeLa cells transfected to over-express surface ULBP1, and (FIG. 11C) cytolysis of HeLa cells expressing non-natural ULBP2.R on their surface. Error bars correspond to standard deviation of technical replicates in the experiment.

FIGS. 12A-12B: MicAbody directed cytolysis of tumor lines by NKG2D-CAR CD8 T cells. (FIG. 12A) Ramos cells—which express CD20, the target of rituximab—were preloaded with calcein and exposed to NKG2D.AF- or NKG2D.YA-CAR cells at an E:T ratio of 20:1 along with increasing concentrations of either ULBP2. S3 or ULBP2.R rituximab-Micabody. The level of cytolysis was quantified after two hours of coincubation. (FIG. 12B) The mouse tumor line CT26 transfected to express human Her2 was used as a cytolysis target in parallel with Ramos cells. NKG2D.AF-CAR CD8 T cells were pre-armed with a saturating concentration (5 nM) of rituximab-ULBP2.R, trastuzumab-ULBP2.R, or an equimolar mixture of the two MicAbodies. Unbound MicAbody was removed by washing and CD8 cells added to target cells at two different E:T ratios. Cytolysis was measure after two hours.

FIGS. 13A-13D: Illustrations of potential MicAbody and MicAdaptor formats. (FIG. 13A) Various antibody Fc variants utilized in developing MicAbody and MicAdaptor reagents and include (a) wild-type human IgG1 Fc, (b) two mutations that render the Fc ADCC-deficient, and (c) electrostatic steering mutations in each Fc—Fc1 or Fc2—that allow for the generation of heterodimeric-Fc molecules which also contains the ADCC-deficiency mutations. (FIG. 13B) Example of how orthogonal ligands can be fused to the C-terminus of the (a) heavy- or (b) light-chain to generate MicAbody reagents. (FIG. 13C) MicAdaptor examples with direct orthogonal ligand fusion without antibody components. (FIG. 13D) Illustration of the variety of MicAdaptor molecules that can be generated in the context of a human IgG1 Fc for enhanced serum stability and may (a, b, c; denoted as “Fc1/Fc2” in text and legends) or may not (d, e) include the heterodimeric-Fc mutations depending upon the valency of the desired molecule for either the heterologous cargo or orthogonal ligand. This may also include utilizing the full antibody structure depending upon desired cargo, valency, and functionality (f, g) and may be either heavy- or light-chain fusions.

FIG. 14: Schematic of CAR constructs, silent CARs, and other CAR variants. SEQ ID NOs for each construct as indicated.

FIGS. 15A-15C: Selective delivery of cytokine fusions to NKG2D.YA-CAR expressing CD8 T cells. (FIG. 15A) CD8 cell expressing either the NKG2D.wt-CAR (SEQ ID NO: 151) or NKG2D.YA-CAR SEQ ID NO: 153) were exposed to 30 or 300 IUe/mL of recombinant human IL2 (rhIL2) or IL15 (rhIL15), or variations of mutant-IL2/-IL15 orthogonal ligand direct fusions or fusions in the context of a heterodimeric Fc (Fc1/Fc2). After three days proliferation was quantified by WST cell proliferation reagent. Data here are shown normalized to the no cytokine control. (FIG. 15B) Human T-cells transduced with a vector encoding NKG2D.YA-CAR were exposed to various ligand-cytokine fusion molecules for seven days and the percentage of GFP+ CAR− cells in the culture tracked over time. (FIG. 15C) Orthogonal ligand enhances delivery of IL21 and mutant-IL21 reagents to NKG2D.YA-CAR cells and promotes their expansion over untransduced cells over three days of culture as determined by WST assay. IUe/mL of cytokine or cytokine-MicAdaptor indicated in parentheses in the abscissa legend. See FIG. 24 for MicAdaptor SEQ ID NOs.

FIGS. 16A-16C: Data exploring sufficiency of the NKG2D.YA ectodomain alone in promoting orthogonal ligand-cytokine-fusion delivery to cells. (FIG. 16A) NKG2D.YA ectodomain alone (SEQ ID NO: 157) was incapable of directing killing of Ramos cells with the rituximab-ULBP2. S3 MicAbody (SEQ ID NOs: 98 and 129). (FIG. 16B) WST proliferation assay after three days of exposure to various cytokine reagents. (FIG. 16C) WST proliferation assay demonstrated that engagement of NKG2D.YA-CAR with orthogonal ligand but in the absence of a fused cytokine or cytokine mutant was insufficient for driving cell expansion. See FIG. 24 for MicAdaptor SEQ ID NOs. IUE/mL amounts of cytokines and cytokine-MicAdaptors indicated in parentheses in abscissa legends.

FIGS. 17A-17C: (FIG. 17A) WST proliferation assay of various costimulatory domain mutants (SEQ ID NOs: 161, 163, and 165) after incubation with the designated cytokine or cytokine-MicAdaptor for three days. (FIG. 17B) Same costimulatory domain CAR mutants examined for their ability to effectively lyse calcein-loaded Ramos target cells in the presence of rituximab-ULBP2. S3 MicAbody (SEQ ID NOs: 98 and 129). (FIG. 17C) Co-expression of the NKG2D.YA ectodomain (NKG2D.YA-ecd) with a complete CD19scFv-CAR is sufficient for promoting proliferation as assessed by WST assay after three days incubation with cytokine reagents. See FIG. 24 for MicAdaptor SEQ ID NOs.

FIG. 18: Summary of candidate, non-natural Fc-eNKG2D variant mutations and protein aggregation properties determined by Size-Exclusion Chromatography (SEC).

FIG. 19: Percent saturation (Rmax) of eNKG2D variants normalized to wild-type NKG2D binding by either MICwed-MicAbody or to MIC25-MicAbody. Wild-type Fc-NKG2D and each Fc-eNKG2D receptor were captured on AHC biosensors then exposed to trastuzumab-specific MicAbodies at 20 nM. Dissociation kinetics were monitored and the Rmax values of the Fc-eNKG2D fusions ranked. Those samples not tested (nt) were due to either severe aggregation or inadequate amount of material expressed or recovered after SEC fractionation.

FIG. 20: EC50 values (nM) for Fc-eNKG2D ELISAs shown in FIG. 3. nt=not tested; nb=no binding or very low binding even at 300 nM so EC50 value not calculated.

FIGS. 21A-21B: Subset of combinatorial mutations within ULBP2 that resulted in phage clones with selective binding to NKG2D.AF versus natural NKG2D.wt as verified by spot ELISA. Mutants were ranked by frequency of appearance among the selected phages.

FIG. 22: Specificity of NKG2D.AF-selected ULBP2 variants in rituximab-MicAbody format retained their binding to NKG2D.AF by quantitative ELISA. The specific amino acid modifications of each ULBP2 variant are shown as are the ratios of their binding to Fc-NKG2D.wt fusion versus Fc-NKG2D.AF fusion. The amino acid residue locations of ULBP2 are those of FIG. 1B.

FIG. 23: Selected mutations at the indicated amino acid locations of ULBP2.R80W (FIG. 1B; SEQ ID NO: 108) that resulted in Y152A-specific phage clones.

FIG. 24: MicAdaptor SEQ ID NOs and Purification Method from Transient Transfections.

### DETAILED DESCRIPTION OF THE INVENTION

Natural killer (NK) cells and certain (CD8+ αβ and γδ) T cells of the immune system have important roles in humans and other mammals as first-line, innate defense against neoplastic and infected cells (Cerwenka, A., and L. L. Lanier. 2001. NK cells, viruses and cancer. Nat. Rev. Immunol. 1:41-49). NK cells and certain T cells exhibit on their surfaces NKG2D, a prominent, homodimeric, surface immunoreceptor responsible for recognizing a target cell and activating the innate defense against the pathologic cell (Lanier, L L, 1998. NK cell receptors. Ann. Rev. Immunol. 16: 359-393; Houchins J P et al. 1991. DNA sequence analysis of NKG2, a family of related cDNA clones encoding type II integral membrane proteins on human NK cells. J. Exp. Med. 173: 1017-1020; Bauer, S et al., 1999. Activation of NK cells and T cells by NKG2D, a receptor for stress-inducible MICA. Science 285: 727-730). The human NKG2D molecule possesses a C-type lectin-like extracellular (ecto-)domain that binds to its eight distinct cognate ligands, the most studied ligands being the 84% sequence identical or homologous, monomeric MICA and MICB, polymorphic analogs of the Major Histocompatibility Complex (MHC) Class I chain-related glycoproteins (MIC) (Weis et al. 1998. The C-type lectin superfamily of the immune system. Immunol. Rev. 163: 19-34; Bahram et al. 1994. A second lineage of mammalian MHC class I genes. PNAS 91:6259-6263; Bahram et al. 1996a. Nucleotide sequence of the human MHC class I MICA gene. Immunogenetics 44: 80-81; Bahram and Spies T A. 1996. Nucleotide sequence of human MHC class I MICB cDNA. Immunogenetics 43: 230-233). Non-pathologic expression of MICA and MICB is restricted to some intestinal epithelium, keratinocytes, endothelial cells and monocytes, but aberrant surface expression of these MIC proteins occurs in response to many types of cellular stress such as proliferation, oxidation and heat shock and marks the cell as pathologic (Groh et al. 1996. Cell stress-regulated human MHC class I gene expressed in GI epithelium. PNAS 93: 12445-12450; Groh et al. 1998. Recognition of stress-induced MHC molecules by intestinal γδ T cells. Science 279: 1737-1740; Zwirner et al. 1999. Differential expression of MICA by endothelial cells, fibroblasts, keratinocytes and monocytes. Human Immunol. 60: 323-330). Pathologic expression of MIC proteins also seems involved in some autoimmune diseases (Ravetch, JV and Lanier L L. 2000. Immune Inhibitory Receptors. Science 290: 84-89; Burgess, S J. 2008. Immunol. Res. 40: 18-34). The differential regulation of NKG2D ligands, such as the polymorphic MICA and MICB, is important to provide the immunity system with a means to identify and respond to a broad range of emergency cues while still protecting healthy cells from unwanted attack (Stephens H A, (2001) MICA and MICB genes: can the enigma of their polymorphism be resolved? Trends Immunol. 22: 378-85; Spies, T. 2008. Regulation of NKG2D ligands: a purposeful but delicate affair. Nature Immunol. 9: 1013-1015).

Viral infection is a common inducer of MIC protein expression and identifies the viral-infected cell for NK or T cell attack (Groh et al. 1998; Groh et al. 2001. Co-stimulation of CD8+αβT cells by NKG2D via engagement by MIC induced on virus-infected cells. Nat. Immunol. 2: 255-260; Cerwenka, A., and L. L. Lanier. 2001). In fact, to avoid such an attack on its host cell, cytomegalovirus and other viruses have evolved mechanisms that prevent the expression of MIC proteins on the surface of the cell they infect in order to escape targeting by the innate immunity system (Lodoen, M., K. Ogasawara, J. A. Hamerman, H. Arase, J. P. Houchins, E. S. Mocarski, and L. L. Lanier. 2003. NKG2D-mediated NK cell protection against cytomegalovirus is impaired by gp40 modulation of RAE-1 molecules. J. Exp. Med. 197:1245-1253; Stern-Ginossar et al., (2007) Host immune system gene targeting by viral miRNA. Science 317: 376-381; Stern-Ginossar et al., (2008) Human microRNAs regulate stress-induced immune responses mediated by the receptor NKG2D. Nature Immunology 9: 1065-73; Slavuljica, I A Busche, M Babic, M Mitrovic, I Gasparovic, D Cekinovic, E Markova Car, EP Pugel, A Cikovic, VJ Lisnic, WJ Britt, U Koszinowski, M Messerle, A Krmpotic and S Jonjic. 2010. Recombinant mouse cytomegalovirus expressing a ligand for the NKG2D receptor is attenuated and has improved vaccine properties. J. Clin. Invest. 120: 4532-4545).

In spite of their stress, many malignant cells, such as those of lung cancer and glioblastoma brain cancer, also avoid the expression of MIC proteins and as a result may be particularly aggressive as they too escape the innate immune system (Busche, A et al. 2006, NK cell mediated rejection of experimental human lung cancer by genetic over expression of MHC class I chain-related gene A. Human Gene Therapy 17: 135-146; Doubrovina, E S, MM Doubrovin, E Vider, RB Sisson, RJ O'Reilly, B Dupont, and YM Vyas, 2003. Evasion from NK Cell Immunity by MHC Class I Chain-Related Molecules Expressing Colon Adenocarcinoma (2003) J. Immunology 6891-99; Friese, M. et al. 2003. MICA/NKG2D-mediated immunogene therapy of experimental gliomas. Cancer Research 63: 8996-9006; Fuertes, MB, MV Girart, LL Molinero, Cl Domaica, LE Rossi, MM Barrio, J Mordoh, GA Rabinovich and NW Zwirner. (2008) Intracellular Retention of the NKG2D Ligand MHC Class I Chain-Related Gene A in Human Melanomas Confers Immune Privilege and Prevents NK Cell-Mediated Cytotoxicity. J. Immunology, 180: 4606-4614).

The high resolution structure of human MICA bound to NKG2D has been solved and demonstrates that the α3 domain of MICA has no direct interaction with the NKG2D (Li et al. 2001. Complex structure of the activating immunoreceptor NKG2D and its MHC class I-like ligand MICA. Nature Immunol. 2: 443-451; Protein Data Bank accession code 1HYR). The α3 domain of MICA, like that of MICB, is connected to the α1-α2 platform domain by a short, flexible linker peptide, and itself is positioned naturally as “spacer” between the platform and the surface of the MIC expressing cell. The three-dimensional structures of the human MICA and MICB α3 domains are nearly identical (root-mean square distance <1 Å on 94 C-αα's) and functionally interchangeable (Holmes et al. 2001. Structural Studies of Allelic Diversity of the MHC Class I Homolog MICB, a Stress-Inducible Ligand for the Activating Immunoreceptor NKG2D. J Immunol. 169: 1395-1400).

T cells, NK-cells, and macrophages can be modified using gene transfer technologies to directly and stably express on their surface binding domains of an antibody that confer novel antigen specificities (Saar Gill & Carl H. June. Going viral: Chimeric Antigen Receptor (CAR) T cell therapy for hematological malignancies. Immunological Reviews 2015. Vol. 263: 68-89; Wolfgang Glienke, Ruth Esser, Christoph Priesner, Julia D. Suerth, Axel Schambach, Winfried S. Wels, Manuel Grez, Stephan Kloess, Lubomir Arseniev and Ulrike Koehl. 2015. Advantages and applications of CAR-expressing natural killer cells. Front. Pharmacol. doi: 10.3389/fphar.2015.00021). CAR-T cells are applications of this approach that combines an antigen recognition domain of a specific antibody along with a fused intracellular domain of the CD3-zeta chain. The CD3-zeta chain is the primary transmitter of signals from the ectodomain of endogenous T cell Receptors (TCRs) to the intracellular space. CARs constructed with the CD3-zeta chain and co-stimulatory molecules such as CD27, CD28, ICOS, 4-1BB, or OX40 trigger CAR-T cell activation upon binding the targeted antigen in a manner similar to an endogenous T cell receptor but independent of the major histocompatibility complex (MHC).

Certain non-natural α1-α2 domains of NKG2D ligands modified to bind the natural human NKG2D receptor with higher affinities than do natural α1-α2 domains have been described (Candice S. E. Lengyel, Lindsey J. Willis, Patrick Mann, David Baker, Tanja Kortemme, Roland K. Strong and Benjamin J. McFarland. Mutations Designed to Destabilize the Receptor-Bound Conformation Increase MICA-NKG2D Association Rate and Affinity. Journal of Biological Chemistry Vol. 282, no. 42, pp. 30658-30666, 2007; Samuel H. Henager, Melissa A. Hale, Nicholas J. Maurice, Erin C. Dunnington, Carter J. Swanson, Megan J. Peterson, Joseph J. Ban, David J. Culpepper, Luke D. Davies, Lisa K. Sanders, and Benjamin J. McFarland. Combining different design strategies for rational affinity maturation of the MICA-NKG2D interface. Protein Science 2012 VOL 21:1396-1402. Herein we describe the attachment of non-natural NKG2D receptors to the surface of mammalian cells in a format that retains the specific binding of modified non-natural NKG2D ligands with attached heterologous molecules, but the non-natural receptors avoid the direct or cis activation of or intracellular signaling to the mammalian cell even when the cell forms an immunologic synapse with a cell or other surface targeted by the heterologous molecule. The non-natural NKG2D receptors themselves have been mutated at one or two specific sites, each of which results in compromised or loss of binding to all natural α1-α2 domains of NKG2D ligands (David J. Culpepper, Michael K. Maddox, Andrew B. Caldwell, and Benjamin J. McFarland. Systematic mutation and thermodynamic analysis of central tyrosine pairs in polyspecific NKG2D receptor interactions. Mol Immunol. 2011 January; 48(4): 516-523; USPTO application 14/562,534; USPTO provisional application 62/088,456)). The instant invention creates CARs that when attached to a mammalian cell surface provide a silenced receptor that can serve as a surrogate high affinity receptor for the attachment to the cell surface of heterologous atoms or molecules Accordingly, via attached non-natural ligands specific for the non-natural modified NKG2D receptor, heterologous molecules comprising a defective cytokine, for example, can be delivered specifically to the silent receptor on the surface of the mammalian cell but not to cells lacking the cognate silent receptor. Once bound to the cell bearing the silent receptor, the defective heterologous molecule may bind to its respective receptor subunits on the cell surface to which binding has been retained and thereby directly signal the cell as if it were stimulated by the wildtype ligand.

Of course, a CAR comprised of an inert non-natural NKG2D, CD3-zeta and costimulatory domain such as CD28, 4-1BB, ICOS, or OX40 on a mammalian cell is capable of directly stimulating and activating the CAR-cell upon forming an immunologic synapse. The activation of such a second or third generation CAR-T cell is dependent upon the function of its CD3-zeta domain and that of at least one costimulatory domain, e.g. 4-1BB or CD28. However, such a CAR can, as a silent CAR, serve as a surrogate high affinity receptor for the binding of cognate non-natural ligand-attached heterologous molecules that have defective binding to their respective natural receptor or receptor subunit(s). This high affinity binding enables the heterologous molecule attached to the non-natural ligand to transmit signals to the cell via their respective other receptor subunits for which binding has been retained.

Importantly, when the CD3-zeta domain of such a direct activation-competent CAR is selectively inactivated, it can still act as a silent CAR and enable the cognate non-natural ligand-attached heterologous molecules that have defective binding to their respective natural receptor or receptor subunit(s) to transmit signals to the cell via their respective other receptor subunits. When the CAR costimulatory domain such as 4-1BB is inactivated and an active CD3− zeta domain is retained, the CAR cannot serve as a silent receptor. That is, although CD3-zeta is not required, a functional costimulatory domain is required to enable a heterologous molecule such as a defective cytokine attached to a cognate non-natural ligand bound to the receptor to mediate its respective signal to the CAR cell.

The instant invention revealed the unexpected need for a costimulatory domain but not CD3-zeta to enable the heterologous defective cytokine attached to a cognate non-natural ligand to mediate its respective signal to the CAR cell. Furthermore, the invention discloses that the costimulatory domain can act in cis or trans to the silent receptor to which is attached the cognate ligand fused to the defective heterologous defective molecule.

When a heterologous molecule such as an antibody or antibody fragment that targets a specific molecule is attached to a cognate non-natural NKG2D ligand which in turn attaches to the silent receptor, the silent receptor-bearing mammalian cell will home to the surface to which the targeting heterologous molecule directs it. Even when a “synapse” is effected between the silent receptor-bearing cell and the targeted cell surface, the former will not be activated by the silent receptor.

Because there are many copies of the non-natural NKG2D-based silent receptors of the instant invention on the cell surface, the homing and/or the selective activation by heterologous molecules can be multiplexed or changed sequentially during manufacturing processes or treatment protocols.

A cell bearing a silent receptor CAR may also express another receptor(s) or CAR orthogonal to the silent CAR and act independently of the silent CAR to specifically and directly activate or otherwise signal that same cell when appropriately stimulated. The other or “second” orthogonal CAR may be a traditional single chain-Fv (scFv)-CAR or a second orthogonal, non-natural modified NKG2D-based CAR with its own cognate non-natural α1-α2 ligand(s). (AF provisional reference). The ability to create effector cells of the immunity system with more than one orthogonal non-natural CAR, silent or active, and multiple cognate non-natural ligands with attached heterologous molecules or atoms, greatly expands the utility, flexibility, and control of Adoptive Cell Therapy (ACT).

In the process of characterizing the silent CAR on a cell and its dependency on a cis or trans acting costimulatory domain, such as 4-1BB, it was observed that compared to an unmodified human T-cell, a human T-cell expressing a silent CAR with a costimulatory domain exhibited a significantly enhanced response to natural IL-2 or to a cognate non-natural ligand fused to either a natural or to a mutant IL-2 with low affinity to its receptor α-subunit. This observation has important utility in the ex vivo or in vivo preferential expansion of cells expressing a CAR comprised of a costimulatory domain with or without a CD3-zeta domain.

As used herein, a “soluble MIC protein”, “soluble MICA” and “soluble MICB” refer to a MIC protein containing the α1-α2 domains with or without α3 domain of the MIC protein but without the transmembrane or intracellular domains. The NKG2D ligands, ULBP1-6, do not naturally possess an α3 domain (Cerwenka A, Lanier L L. 2004. NKG2D ligands: unconventional MHC class I-like molecules exploited by viruses and cancer. Tissue Antigens 61 (5): 335-43. doi:10.1034/j.1399-0039.2003.00070.x. PMID: 12753652). An “α1-α2 domain” of an NKG2D ligand refers to the protein domain of the ligand that binds an NKG2D receptor.

In some embodiments, the α1-α2 domains of the non-natural NKG2D ligand proteins of the invention are at least 80% identical or homologous to the native or natural α1-α2 domain of an NKG2D ligand(SEQ ID NOs: 1-9 for MICA, MICB, ULBP1, ULBP2, ULBP3, ULBP4, ULBP5, ULBP6, and OMCP, respectively). In other embodiments, the modified α1-α2 domain is 85% identical to a native or natural α1-α2 domain of an NKG2D ligand. In yet other embodiments, the modified α1-α2 domain is 90% identical to a native or natural α1-α2 domain of a natural NKG2D ligand protein and binds non-natural NKG2D.

Preferably the modified or non-natural α1-α2 domains of the non-natural MIC proteins of the invention are at least 80% identical or homologous to a native or natural α1-α2 domain of one of the 8 human NKG2D ligand proteins (SEQ ID NOs: 1-8) and bind the non-natural NKG2D ectodomain. In some embodiments, the non-natural α1-α2 domain is 85% identical to a native or natural α1-α2 domain of an NKG2D ligand protein and binds the non-natural NKG2D. In other embodiments, the non-natural α1-α2 platform domain is 90%, 95%, 96%, 97%, 98%, or 99% identical to a native or natural α1-α2 platform of a human natural α1-α2 domain protein and binds the non-natural NKG2D.

In some embodiments, a heterologous molecular tag may be fused to the N-terminus or C-terminus of a non-natural α1-α2 domain of a soluble MIC protein or to that of an attached heterologous peptide or protein to aid in the purification of the soluble ligand. Tag sequences include peptides such as a poly-histidine, myc-peptide, a FLAG tag, streptavidin-like tag, or a small molecule such as biotin. Such tags may be removed after isolation of the MIC molecule by methods known to one skilled in the art.

Specific mutations in α1-α2 domains of NKG2D ligands can be made to create non-natural α1-α2 domains that bind non-natural NKG2D receptors, themselves engineered so as to have reduced affinity for natural NKG2D ligands. This can be done, for example, through genetic engineering. A non-natural NKG2D receptor so modified can be used to create on the surface of NK-cells, T cells, macrophages or other cells of the immune system an NKG2D-based CAR that can bind to molecules comprised of the non-natural α1-α2 domains. These non-natural NKG2D receptors and their cognate non-natural NKG2D ligands will provide important safety, efficacy, and manufacturing advantages for treating cancer and viral infections as compared to the current CAR-T cells and CAR-NK cells, as described below. When the intracellular signaling of non-natural NKG2D receptors on the surface of mammalian cells has been silenced as in the instant invention, these invented CARs can act as surrogate high affinity receptors for otherwise defective heterologous molecules such as cytokines, chemokines, lymphokines, cytotoxins, and atoms fused or conjugated to the orthogonal NKG2D ligands. This provides delivery of the heterologous molecules directly and specifically to the silent receptor-bearing cell without the silent-receptor per se directly activating its host cell. Furthermore, heterologous molecules that bind specific targets and thereby cells or other surfaces bearing such targets can provide specific homing functions to the silent receptor-bearing cell without its unintended activation or stimulation.

CAR-T or CAR-NK cells comprised of ectodomains of non-natural NKG2D receptors that do not or only poorly bind natural NKG2D ligands will not be subject to activation by any natural ligands and thus will not be toxigenic as are cells expressing a CAR based on a natural NKG2D receptor. Furthermore, ectodomains of non-natural NKG2D receptors on cells will not be subject to down-regulation by natural NKG2D ligands in a soluble format or on Myeloid Derived Suppressor Cells (MDSC) (Deng W, Gowen B G, Zhang L, Wang L, Lau S, lannello A, Xu J, Rovis T L, Xiong N, Raulet D H, 2015. Antitumor immunity. A shed NKG2D ligand that promotes natural killer cell activation and tumor rejection. Science. 2015 Apr. 3; 348(6230):136-9. doi: 10.1126/science.1258867. Epub 2015 Mar. 5). However, when such CAR cells bearing ectodomains of non-natural NKG2D receptors are engaged by bispecific molecules with the cognate non-natural α1-α2 domains of the instant invention and its heterologous targeting motif which has found and bound its intended target, the CAR will be activated and the CAR-cell's effector functions expressed.

Because the CAR-T or CAR-NK cells comprised of non-natural NKG2D receptor ectodomains are not activated except in the presence of an engaged bispecific molecule comprised of a cognate non-natural α1-α2 domain, their activation can be controlled by the administered bispecific molecules, which as biopharmaceuticals will exhibit pharmacokinetics and pharmacodynamics well known in the field. In the event that an adverse event develops, the physician can simply modify the dosing regimen of the administered bispecific molecule rather than having to deploy an induced suicide mechanism to destroy the infused CAR cells as currently done (Monica Casucci and Attilio Bondanza. Suicide Gene Therapy to Increase the Safety of Chimeric Antigen Receptor-Redirected T Lymphocytes. J Cancer. 2011; 2: 378-382). Furthermore, such bispecific molecules with different specific targeting motifs can be administered simultaneously or sequentially to help address tumor resistance and escape as a results of target antigen loss without having to create, expand and infuse multiple different autologous CAR cells (Gill & June, 2015). Since all CAR constructions can be identical for all CAR cells and the targeting specificity determined simply by the targeting motif of the administered bispecific molecule of the instant invention, manufacturing processes will be simplified and less expensive.

Examples of parent or recipient proteins or polypeptides that are candidates for attachment to non-natural α1-α2 domains of NKG2D ligands include but are not limited to antibodies, proteins comprised of Ig folds or Ig domains, including modified Fc domains that recruit natural molecules or fail to recruit or bind natural molecules, globulins, albumens, fibronectins and fibronectin domains, integrins, fluorescent proteins, enzymes, outer membrane proteins, receptor proteins, T cell receptors, chimeric antigen receptors, viral antigens, virus capsids, viral ligands for cell receptors, hormones, cytokines and modified cytokines such as interleukins, knottins, cyclic peptides or polypeptides, major histocompatibility (MHC) family proteins, MIC proteins, lectins, and ligands for lectins. It is also possible to attach non-protein molecules such a polysaccharides, dendrimers, polyglycols, peptidoglycans, antibiotics, and polyketides to the modified α1-α2 domains of NKG2D ligands.

Thus, the instant invention expands the diversity and practicality of this remarkable, very promising immunologic approach to managing cancer with CAR-T cells, CAR-NK cells, and CAR-macrophage-like cells while overcoming many of these current, recognized difficulties.

As used herein “peptide”, “polypeptide”, and “protein” are used interchangeably; and a “heterologous molecule”, “heterologous peptide”, “heterologous sequence” or “heterologous atom” is a molecule, peptide, nucleic acid or amino acid sequence, or atom, respectively, that is not naturally or normally found in physical conjunction with the subject molecule. As used herein, “non-natural” and “modified” are used interchangeably. As used herein, “natural”, “native”, and “wild-type” are used interchangeably and “NKG2D” and “NKG2D receptor” are used interchangeably. The term “antibody” herein is used in the broadest sense and specifically covers monoclonal antibodies, multispecific antibodies (e.g. bispecific antibodies), and antibody fragments, so long as they exhibit the desired biological activity. “Antibody fragments” comprise a portion of an antibody, preferably comprising the antigen binding region thereof. Examples of antibody fragments include Fab, Fab′, F(ab′)₂, Fv fragments and insertible Fv's; diabodies; linear antibodies; single-chain antibody molecules; and multi-specific antibodies formed from antibody fragment(s).

The term “comprising,” which is used interchangeably with “including,” “containing,” or characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

All references cited herein are hereby incorporated by reference in their entireties, whether previously specifically incorporated or not. As used herein, the terms “a”, “an”, and “any” are each intended to include both the singular and plural forms.

Having now fully described the invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and may be applied to the essential features hereinbefore set forth.

### EXAMPLES

### Modified NKG2D Receptor Ectodomain and Modified α1-α2 Domains of NKG2D Ligands

### Example 1. Modification of Tyrosine 152 to Alanine (Y152A) and Tyrosine 199 to Phenylalanine (Y199F) of the Human NKG2D Receptor to Create an Inert NKG2D Ectodomain

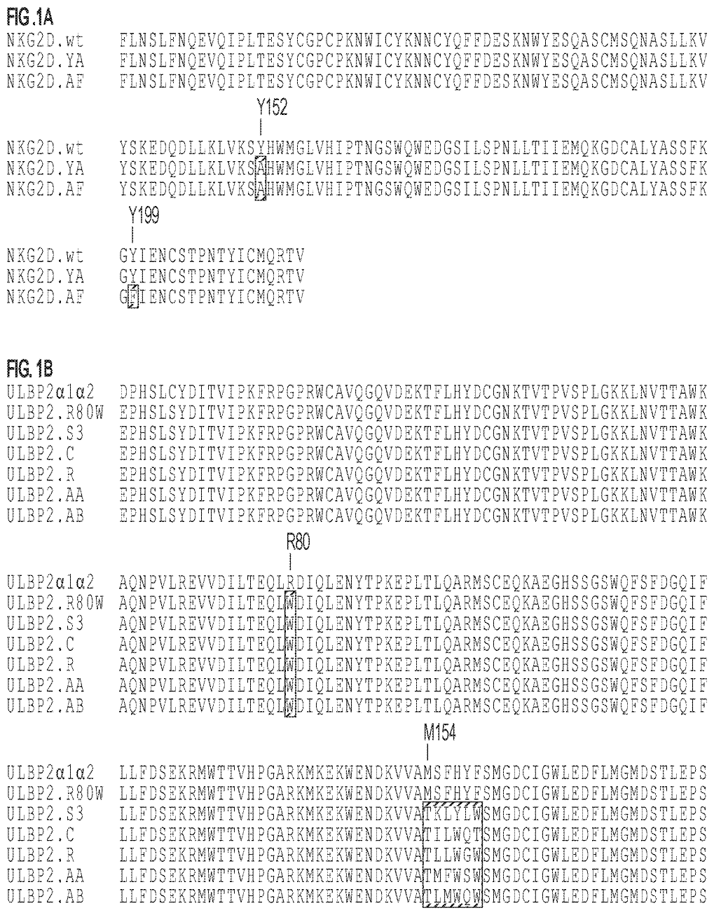

It had been demonstrated by others that mutations at tyrosine 152 or at tyrosine 199 in human NKG2D, the equivalent of positions 73 and 120 of the NKG2D ectodomain (FIG. 1A, SEQ ID NO.:17) can greatly reduce binding to the natural ligand, MICA (David J. Culpepper, Michael K. Maddox, Andrew B. Caldwell, and Benjamin J. McFarland. Systematic mutation and thermodynamic analysis of central tyrosine pairs in polyspecific NKG2D receptor interactions. Mol Immunol. 2011 January; 48(4): 516-523). We reasoned that while mutation of either tyrosine residue greatly affected the ability of NKG2D to bind to its natural ligands, simultaneous mutation at both tyrosine 152 (Y152) and tyrosine 199 (Y199) would virtually eliminate the receptor's ability to engage with all native ligands. We therefore sought to explore individual and combinatorial Y152 and Y199 substitutions and characterize them with regard to their biochemical behavior with the objective of identifying both single and double-mutant variants incapable of engaging any natural ligands. Those variants that also expressed and assembled well were of particular interest as these signified inert ligands that could be more easily produced for analysis.

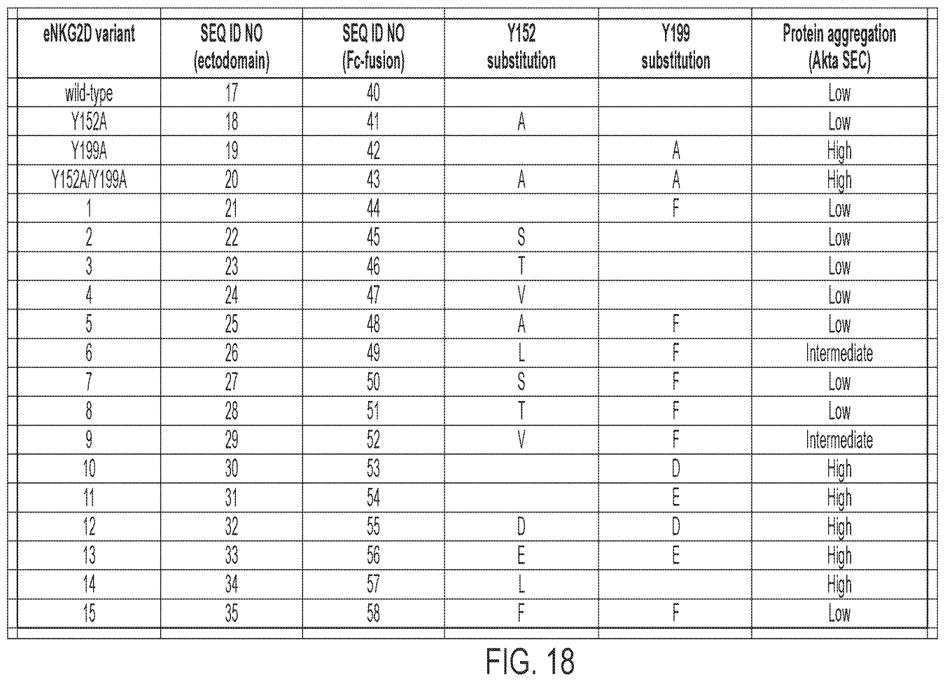

Natural NKG2D (wild-type) ectodomain (NKG2D.wt, SEQ ID NO: 17) and candidate non-natural NKG2D variant ectodomains (SEQ ID NOs: 18-35)—also termed “engineered NKG2D” or “eNKG2D” were cloned as fusions to the C-terminus of human IgG1 Fc (without Fab domains), via a short factor Xa recognizable Ile-Glu-Gly-Arg linker (SEQ ID NO: 38) and are interchangeably referred to as Fc-NKG2D.wt or NKG2D.wt and Fc-eNKG2D or eNKG2D (SEQ ID NOs: 40-58). gBlocks' DNA Fragments (Integrated DNA Technologies, San Diego, CA), corresponding to the MHCI signal sequence (SEQ ID NOs: 36 and 37), human IgG1 Fc with linker (SEQ ID NO: 39), and NKG2D ectodomain variants (SEQ ID NOs: 59-77) were synthesized and inserted into pD2610-V12 (ATUM, Newark, CA). DNA constructs exploring substitutions at Y152, Y199, or a combination of Y152/Y199 mutations (FIG. 18) were expressed transiently in Expi293™ cells (ThermoFisher Scientific, Waltham, MA) and secreted protein purified by Protein A affinity chromatography (cat. no. 20334, Pierce Biotechnology, Rockford, IL). Eluted material was characterized by size-exclusion chromatography (SEC) on Akta Pur Superdex columns and correctly assembled, size-appropriate material was fractionated and isolated from aggregate peaks prior to inclusion in assays.

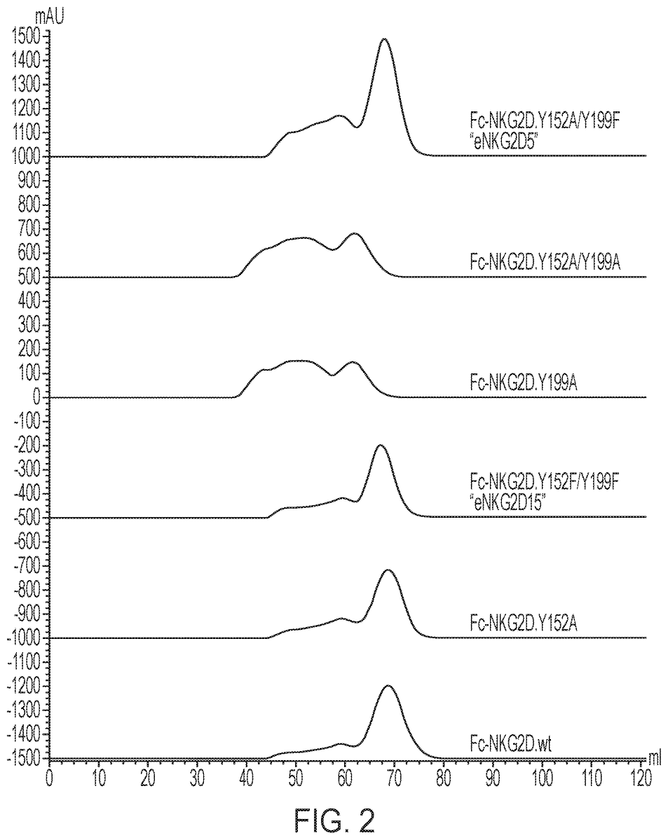

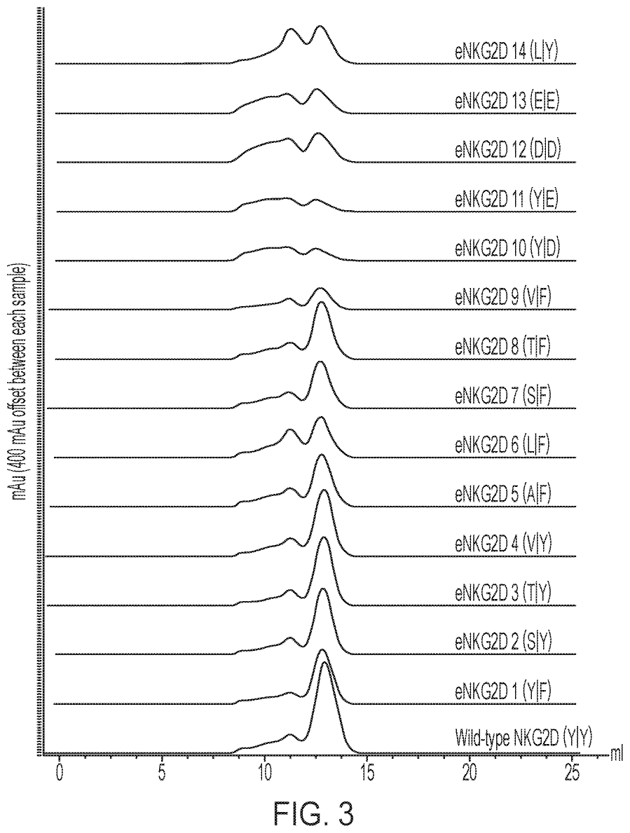

SEC characterization of purified NKG2D.Y199A-Fc fusion revealed a composition of predominantly aggregated material (FIG. 2). In comparison, both the natural Fc-NKG2D fusion and Fc-NKG2D.Y152A fusion material were distinguished by a discrete, non-aggregated peak that was readily differentiated from more rapidly migrating aggregate. The effect of the Y199A mutation on aggregation was also apparent in the Y152A/Y199A double-mutant Fc-NKG2D fusion variant, indicating that it had an overriding influence on protein misfolding (FIG. 2). This aspect of including Y199A with any combination of Y152 mutations in NKG2D variants therefore presented a challenge for the production of material necessary for subsequent engineering efforts and raised a concern about assembly and presentation on a cell surface. As a consequence, an effort was made to explore other substitutions at Y152 and Y199 that could be combined to yield a more robust molecule. eNKG2D combinatorial Y152 and Y199 mutant candidates were examined as Fc fusions and detailed in (FIG. 18). In addition, all purified and expressed Fc-eNKG2D fusion candidates were profiled by SEC and their chromatograms revealed varying levels of aggregate formation (FIGS. 2 and 3, FIG. 18). Of the single amino acid substitutions explored at residue 152 alanine, serine, threonine, and valine all did not impact assembly of the Fc-NKG2D molecule although Y152-leucine (Y152L) resulted in highly aggregated material. Similar to alanine, neither glutamate nor aspartate were tolerated at position 199, although phenylalanine only modestly increased aggregate formation. Of the combinations of mutations that were explored, Y152A/Y199F, Y152S/Y199F, Y152T/Y199F, and Y152F/Y199F did not negatively impact the desired dimer formation, whereas other combinations resulted in increased aggregation (FIG. 18, FIGS. 2 and 3).

### Example 2: Generation of Antibody-Based Bispecific Molecules, “MicAbodies”, with Non-Natural NKG2D Ligand Variants

To generate non-natural MicA variants fused to human IgG1, the DNA polynucleotides encoding the α1-α2 domains of, for example, MICwed (SEQ ID NO: 79) and MIC25 (SEQ ID NO: 81), were PCR amplified using primers that also introduced the polynucleotide encoding either an APTSSSGGGGS linker for fusion to C-terminal kappa light chain (SEQ ID NO: 84) or for a GGGS linker for fusion to C-terminal heavy chain of human IgG1 (SEQ ID NO: 82). Furthermore, two mutations were introduced into the CH2 domain of the heavy chain—D265A/N297A (Kabat numbering; FIGS. 13A and 13B)—that reduce binding to all FcγR receptors thus eliminating antibody-dependent cell cytotoxicity (ADCC) function (Shields et al., 2001 JBC, 276:6591-6604). The polynucleotide encoding the α1-α2 domain of wild-type ULBP2 (ULBP2.wt) without its GPI-linkage (SEQ ID NO: 12) was similarly cloned and fused to the DNA polynucleotides encoding the linkers and the IgG1 heavy chain or light chain. These bispecific antibodies termed “MicAbody™” in the singular, “MicAbodies” in the plural—are bivalent for the fused α1-α2 domain. Examples of antibodies used to generate MicAbodies for the purposes of exploring eNKG2D engineering include but were not limited to trastuzumab (SEQ ID NOs: 94 and 96) and ritixumab (SEQ ID NOs: 98 and 100) and subsequently termed “trastuzumab-MicAbody” (e.g. SEQ ID NOs: 102 and 104) and “rituximab-MicAbody” (e.g. SEQ ID NO: 106), respectively. The fusion constructs were inserted individually into pD2610-V12 (ATUM, Newark, CA) via Gibson cloning (New England Biolabs Inc., Ipswich, MA). For a given antibody recognizing a specific antigen, the plasmid encoding the heavy chain and the plasmid encoding the light chain fused to either natural or non-natural NKG2D ligand were co-transfected for transient expression in Expi293™ cells (ThermoFisher Scientific, Waltham, MA). Alternatively, the plasmid encoding the heavy chain fused to either natural or non-natural NKG2D ligand and the plasmid for light chain were co-transfected. Secreted bispecific antibodies were purified by Protein A affinity chromatography (cat. no. 20334, Pierce Biotechnology, Rockford, IL), eluted material was characterized by size-exclusion chromatography (SEC) on Akta Pur Superdex columns, and fractionation performed as needed. In addition, SDS-PAGE analysis was performed on purified samples to verify the expected molecular weights of the fused heavy chain and fused light chain species.

### Example 3: Identifying Modified NK2GD Variants Incapable of Binding to Either Natural NKG2D-Binding Ligands or to Non-Natural Ligands that have Enhanced Binding to Wild-Type NKG2D

The binding affinities of α1-α2 variants to the extracellular domains of natural (wild-type) NKG2D and non-natural eNKG2D proteins were analyzed using a plate-based ELISA method. Each of the SEC fractionated natural Fc-NKG2D and non-natural Fc-eNKG2D fusions were coated overnight at 4° C. onto separate wells of Nunc Maxisorp 96 well plates (Thermo Fisher Scientific, Waltham, MA) using a coating concentration of 1 pg/mL in phosphate-buffered saline (PBS). The plates were washed three times in PBS/0.05% Tween-20 (PBS-T) at 20-22° C., and blocked with 0.5% bovine serum albumin in PBS (PBS-B) for 2 hours at 20-22° C. MicAbodies were titrated against the plate-bound natural or non-natural Fc-NKG2D fusions for 60 minutes at 20-22° C. in PBS/0.5% bovine serum albumin (BSA)/0.05% Tween-20 (PBS-BT), washed 3 times with PBS-T at 20-22° C., and the bound bispecific proteins detected using an HRP-conjugated anti-human kappa in PBS-BT (Abcam, Cambridge MA) and developed with 1-Step™ Ultra TMB ELISA Substrate Solution (Thermo Fisher Scientific, Waltham, MA). The binding of the ULBP2.wt rituximab-MicAbody (SEQ ID NOs: 98 and 106) discriminated between wild-type NKG2D and eNKG2D variants with reduced binding to the latter, and ligand variants—MICwed (SEQ ID NOs: 96 and 102) and MIC25 (SEQ ID NOs: 96 and 104)—were more stringent at identifying eNKG2D variants with abolished ligand binding. The binding behaviors for each eNKG2D variant against all three bispecific ligands revealed the combinations of NKG2D modifications that led to the greatest reduction in binding of wild-type and variant ligands and enabled the selection of lead inert NKG2D variants.

Additional biophysical analysis of eNKG2D variant binding to ligands was also performed with Bio-Layer Interferometry (BLI) using the ForteBio Octet system (all ForteBio LLC, Fremont, CA). For these experiments human NKG2D ligands MICA-Fc, MICB-Fc, ULBP1-Fc, ULBP2-Fc, ULBP3-Fc, and ULBP4-Fc were purchased from R&D Systems, Inc. (Minneapolis, MN). Ligands in the MicAbody format were captured on anti-human IgG Fc capture (AHC) biosensor tips. After a baselines were established, tips were exposed to a titration series of Fc-eNKG2D fusion proteins ranging from 300 nM to 0.41 nM and association/dissociation kinetics monitored with all steps performed in PBS-BT. Subsequently, Fc-eNKG2D fusion proteins were captured onto AHC tips and MicAbodies were titrated to characterize binding kinetics.

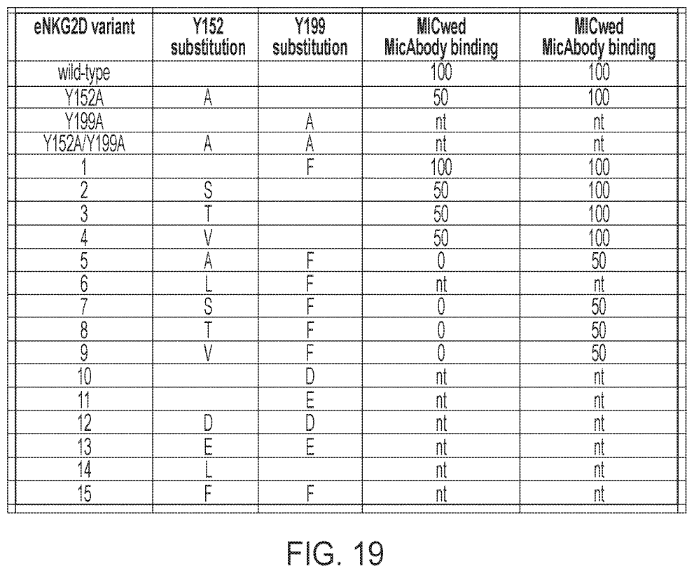

To determine the maximum response as defined by binding of natural NKG2D to either MlCwed or MIC25, natural Fc-NKG2D fusions were captured onto AHC biosensors and 20 nM trastuzumab-MlCwed or 20 nM trastuzumab-MIC25 MicAbodies were incubated for two minutes and then dissociation kinetics observed for 30 seconds. Binding analysis under the same conditions was then performed with Fc-eNKG2D fusion receptors as the capture agent, and the level of binding for each eNKG2D ranked as a percentage of the maximal binding response established by Fc-NKG2D.wt (FIG. 19). For MlCwed, the responses of all single mutant Fc-eNKG2D variants, except for Y199F, were diminished to 50%. Y199F maintained 100% binding response. However, all double-mutant Fc-eNKG2D variants had completely abolished binding to MlCwed. For MIC25, all single mutant Fc-eNKG2D variants and Y152V/Y199F maintained 100% binding response relative to wild-type Fc-NKG2D binding. However, binding was reduced to 50% with several of the double-mutant Fc-eNKG2D variants including Y152A/Y199F, Y152S/Y199F, and Y152T/Y199F.

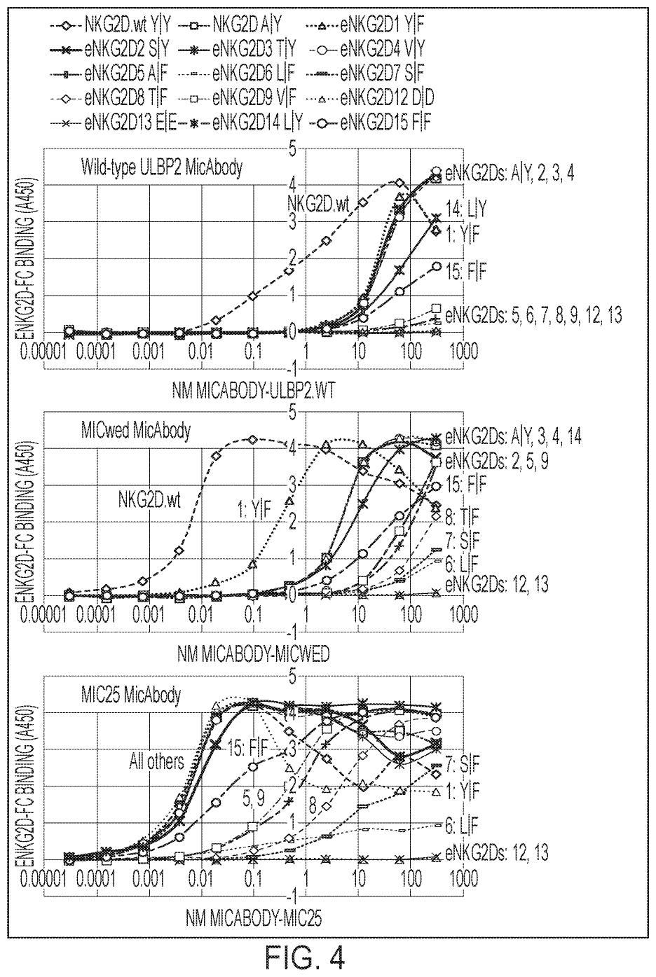

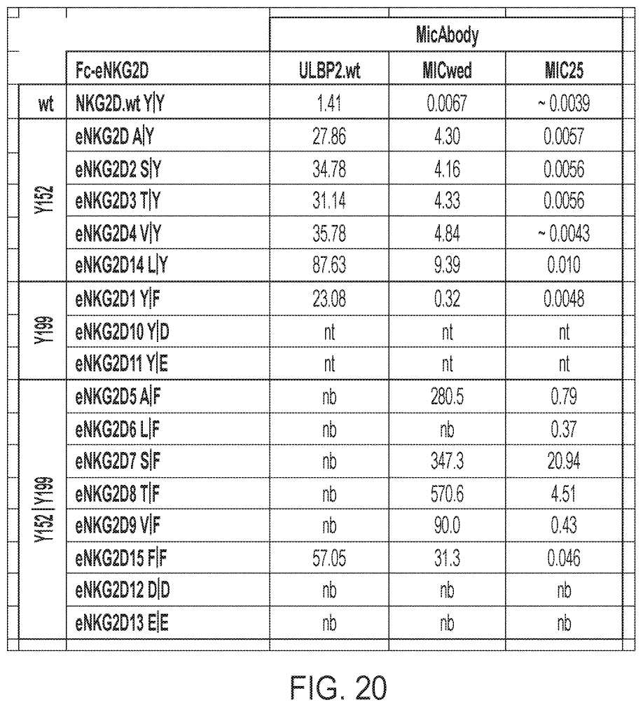

ELISA assays with Fc-eNKG2D fusions as capture agents were performed with ULBP2.wt, MlCwed, MIC25 MicAbodies titrated starting at 300 nM (FIG. 4). EC₅₀ values were calculated when possible using GraphPad Prism (FIG. 20). Natural NKG2D bound to ULBP2, MlCwed, and MIC25-based MicAbodies with affinities calculated as Kds values of 1.4, 0.007, and 0.005 nM, respectively. While affinity was diminished for ULBP2 and MlCwed MicAbodies with all single mutant eNKG2D candidates, binding of MIC25 to eNKG2D candidates was retained. However, all double-mutant eNKG2D candidates had eliminated or significantly reduced binding to all three ligands—ULBP2, MlCwed, and MIC25—in Micabody formats.

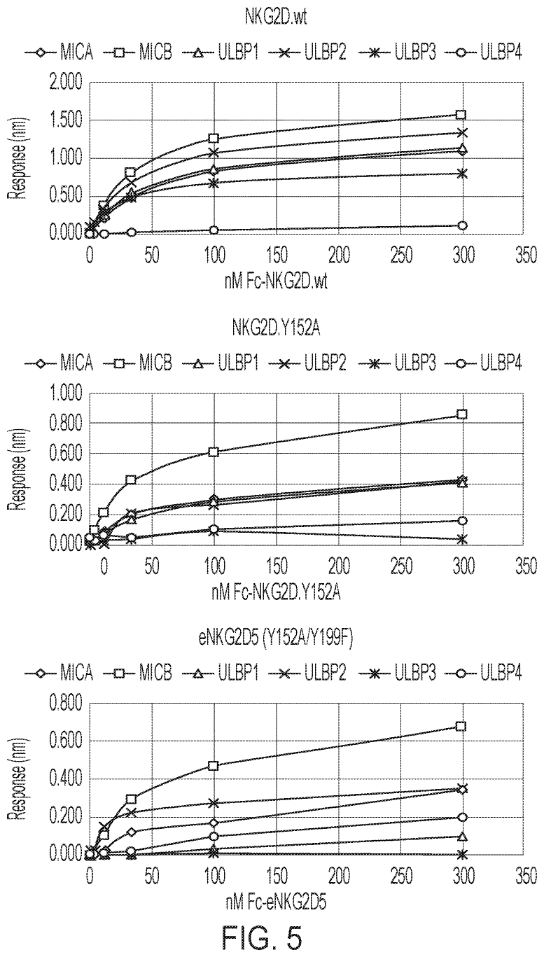

eNKG2D variants eNKG2D5 (Y152A/Y199F), eNKG2D7 (Y152S/Y199F), eNKG2D8 (Y152T/Y199F), and eNKG2D9 (Y152V/Y199F) had reduced or abolished binding to ULBP2, MlCwed, and MIC25-based MicAbodies by both Octet analysis and ELISA (FIGS. 19 and 20). Furthermore, eNKG2Ds 5, 7, and 8 had the least amount of aggregation, suggestive of more robust protein assembly upon 293T expression (FIG. 18). eNKG2D5 (SEQ ID NO: 48) was examined more closely for binding to wild-type ligands as MicAbodies captured on Octet AHC tips. Single mutant Fc-NKG2D.Y152A (SEQ ID NO: 41) had reduced binding to all natural ligands relative to natural (SEQ ID NO: 40) NKG2D (FIG. 5). The response curve for binding of eNKG2D5 (Y152A/Y199F) was reduced even further relative to Y152A eNKG2D. eNKG2D5 (Y152A/Y199F, henceforth referred to as “AF” or “NKG2D.AF”) was chosen as the lead NKG2D variant for which to engineer cognate selective, orthogonal, non-natural ligands.

### Example 4: Constructing Orthogonal Non-Natural α1-α2 Domains with Selective Binding to Non-Natural NKG2D.AF Ectodomain

We employed phage display to engineer orthogonal non-natural α1-α2 domains that exhibit selective binding to the NKG2D.AF (SEQ ID NO: 48) receptor. As a starting point, the non-natural ULBP2.R80W α1-α2 domain (FIG. 1B; SEQ ID NO: 108) with high affinity for natural, wild-type NKG2D (NKG2D.wt) ectodomain was selected as the parent domain for further mutagenesis and screening by phage display. Synthetic DNA libraries were generated for the α1-α2 domain of ULBP2.R80W (SEQ ID NO: 108) which additionally has a C8S mutation to eliminate the potential for disulfide linkages. Codons of amino acid residues of the ligand that in the bound state are positioned in close proximity to the Y152 and Y199 positions on the natural NKG2D receptor were replaced with NNK codons; the libraries consisted of NNK codons at positions 154-159 (FIG. 1B; SEQ ID NO: 110). Libraries were cloned as fusions to the pill minor coat protein of M13 phage, and phage particles displaying the mutagenized α1-α2 domain variants were produced in SS320 E. coli cells according to standard methodologies (Andris-Widhopf, J., Steinberger, P., Fuller, R., Rader, C., and Barbas, C. F., 3rd. (2011). These α1-α2 phage display libraries were sorted for high binding affinity to the non-natural NKG2D.AF receptor by selectively capturing phage clones bound to biotinylated Fc-NKG2D.AF protein in the presence of non-biotinylated natural Fc-NKG2D.wt competitor protein. Selective clones were enriched by cycling through multiple rounds of competitive selection with increasing concentrations of the non-biotinylated natural Fc-NKG2D.

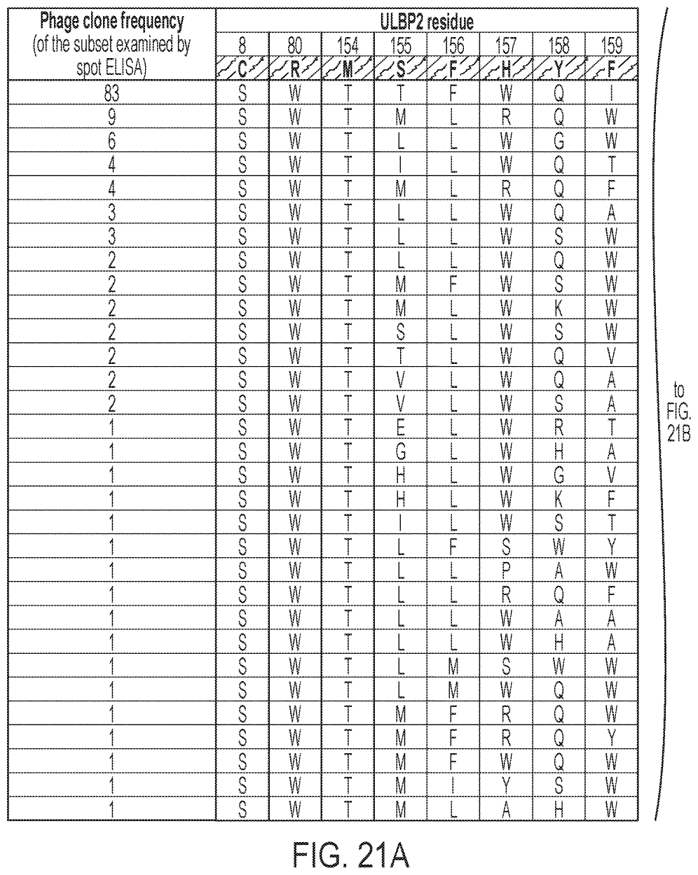

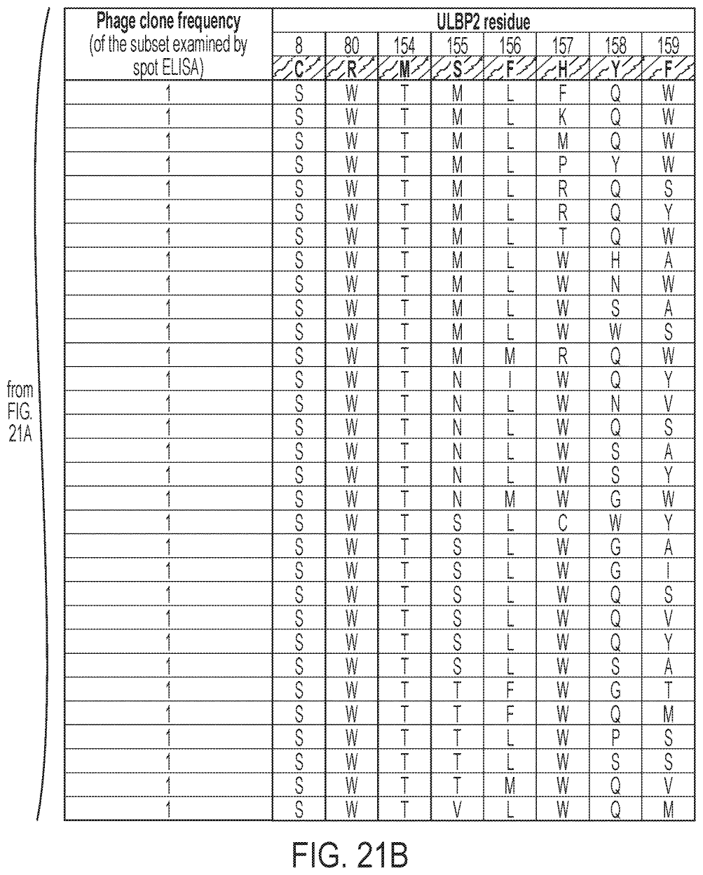

After four rounds of selection, phage clones were individually arrayed in 96-well format, spot ELISAs were performed to verify preferred differential binding to plate-bound non-natural NKG2D.AF versus NKG2D.wt. Bound phages were detected with biotinylated M13 phage coat protein monoclonal antibody E1 (ThermoFisher Scientific, Waltham, MA), streptavidin-HRP detection (R&D Systems, Minneapolis, MN), and 1-Step Ultra TMB ELISA development (ThermoFisher Scientific, Waltham, MA). The spot ELISA signal for each clone was expressed as a ratio of phage binding NKG2D.AF to phage binding NKG2D.wt. Those phages with a ratio greater than or equal to 14 were sequenced to identify the specific mutations within the NNK mutagenized regions. FIGS. 21A-21B show the selected amino acid residues for each α1-α2 phage variant that selectively bound NKG2D.AF. In instances where multiple clones representing the same sequence were identified, the ratio of ELISA signals was plotted, and consistency of phage clones was verified by the clustering of data points (data not shown).

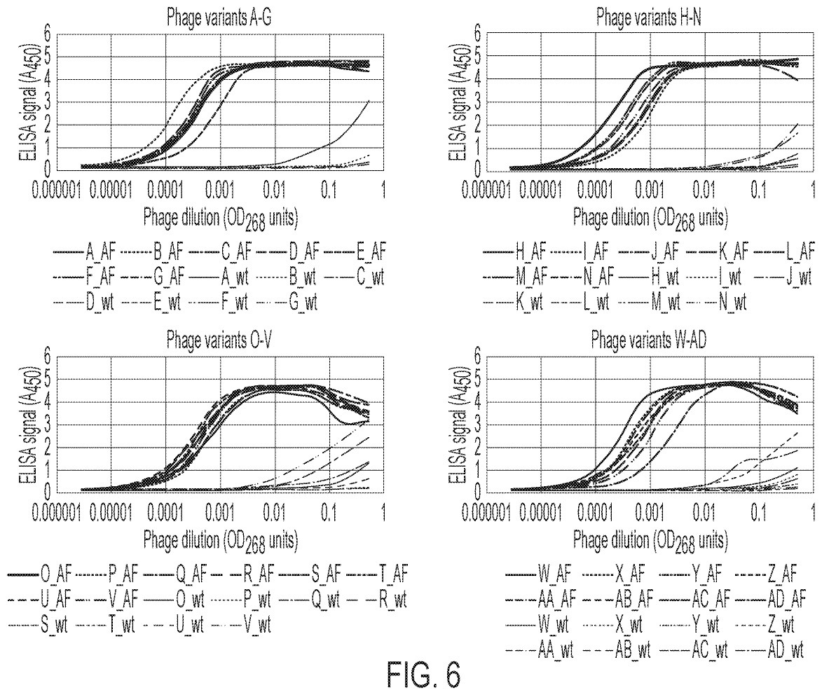

Thirty of the variants identified in ELISAs were expanded in individual monocultures to generate high titer microbatches of phage. Purified phage concentrations were normalized to an OD₂₆₈=0.5 then subject to 1:3 dilution series against plate-bound Fc-NKG2D.AF or Fc-NKG2D.wt with phage detection and ELISA development performed as described above. All thirty variants assayed in this manner consistently demonstrated selective binding to NKG2D.AF with little to no binding to NKG2D.wt (FIG. 6) even at the highest concentrations of phage assayed. The selected phages also exhibited a shift of two or more logs of phage concentration to achieve half-maximal binding between NKG2D.AF and NKG2D.wt.

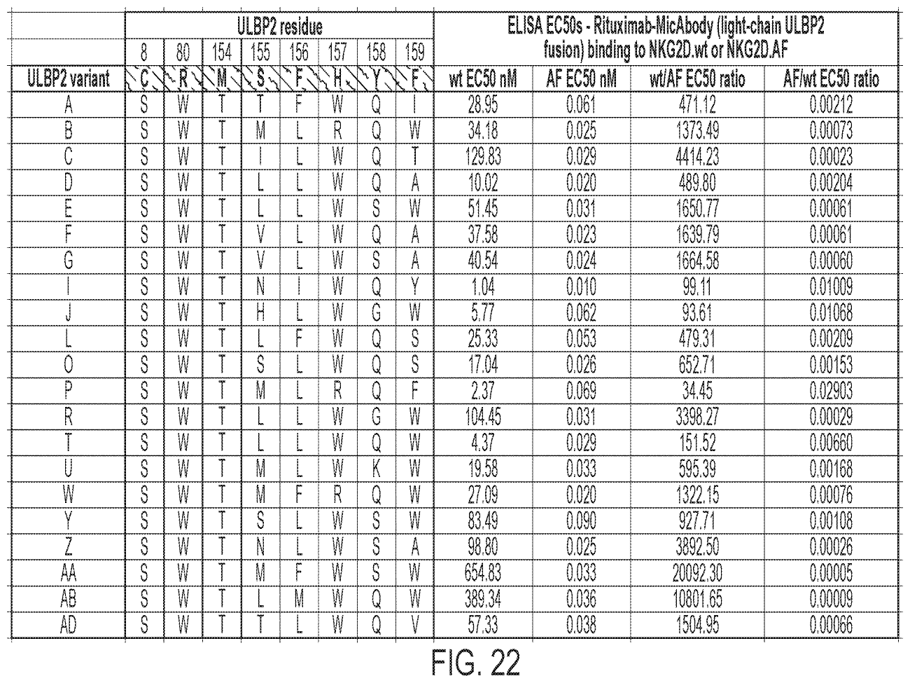

To confirm that the NKG2D.AF-selective α1-α2 domain variants retained specific binding properties in the context of antibody fusions, 21 variants (FIG. 22; e.g. SEQ ID NOs: 111-118) were cloned as C-terminal fusions with an APTSSSGGGGS linker to the light chain of the rituximab antibody (SEQ ID NOs: 119-126). The resulting fusions were cloned into the mammalian expression vector pD2610-V12 (ATUM, Newark, CA) via Gibson cloning (New England Biolabs Inc., Ipswich, MA) and co-expressed with the heavy chain of the parent antibody (SEQ ID NO: 99) as paired full IgG antibodies. Transient expressions were carried out in Expi293™ cells (ThermoFisher Scientific, Waltham, MA) according to the manufacturer's protocol, and purified using standard protein-A affinity chromatography (cat. no. 20334, Pierce Biotechnology, Rockford, IL). ELISAs measuring the binding of each variant ULBP2 α1-α2 antibody fusions to non-natural Fc-NKG2D.AF and to natural Fc-NKG2D.wt demonstrated their significantly greater binding affinity toward NKG2D.AF relative to the natural NKG2D.wt (FIG. 22). Collectively, these data demonstrated the invention of non-natural, orthogonal α1-α2 domains that possessed high affinity binding to the non-natural NKG2D.AF receptor and significantly reduced binding affinity to the natural NKG2D receptor. Furthermore, fusions of these orthogonal α1-α2 domains to antibody polypeptides retained their selective binding properties and were used, for example, in the context of chimeric antigen receptor (CAR) T cells, to redirect non-natural NKG2D.AF receptors toward specific antigens.

### Example 5: Identifying Non-Natural NKG2D Ligands that can Discriminate Between Non-Natural NKG2D Receptor Variants by Selectively Binding One or the Other

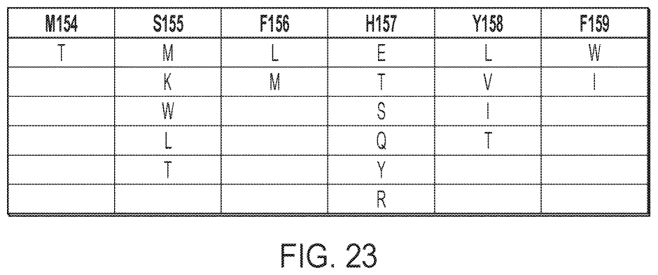

Phage display to engineer orthogonal non-natural α1-α2 domains with selective binding to NKG2D.Y152A (henceforth referred to as NKG2D.YA, receptor was performed with non-natural ULBP2.R80W α1-α2 domain (SEQ ID NO: 108) as the starting point as described above. The α1-α2 phage display libraries were panned for high binding affinity to the non-natural Fc-NKG2D.YA receptor by selectively capturing phage clones bound to biotinylated Fc-NKG2D.YA (SEQ ID NO: 41) protein in the presence of non-biotinylated natural Fc-NKG2D.wt (SEQ ID NO: 40) competitor protein. Additional phage clone validation work resulted in the identification of variants with preferential binding to Fc-NKG2D.YA versus Fc-NKG2D.wt (FIG. 23). ULBP2. S3 (SEQ ID NO: 127), for example, consistently demonstrated selective binding by ELISA and Octet analysis (both in monomeric His-tagged and bispecific antibody fused format) to non-natural NKG2D.YA relative to natural NKG2D.wt. This represented a distinct form of the invention of non-natural orthogonal α1-α2 domains possessing high affinity binding to non-natural NKG2D receptors (in this case NKG2D.YA as opposed to NKG2D.AF as in Example 4). Furthermore, fusions of orthogonal α1-α2 domains to antibody polypeptides retained their selective binding properties and were used to selectively redirect non-natural NKG2D receptors towards specific molecules determined by fused heterologous peptides such as antibodies.

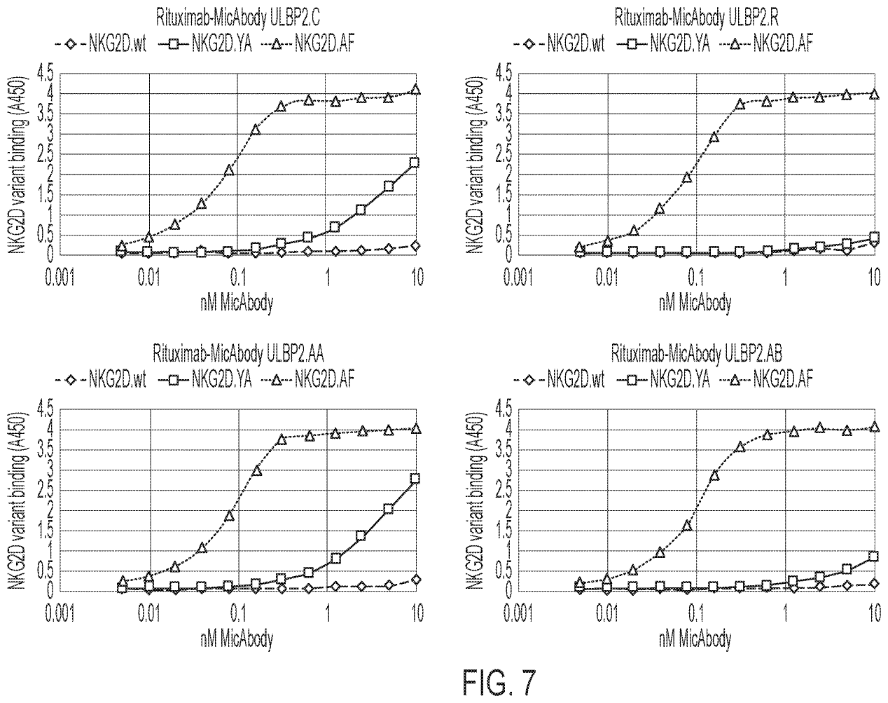

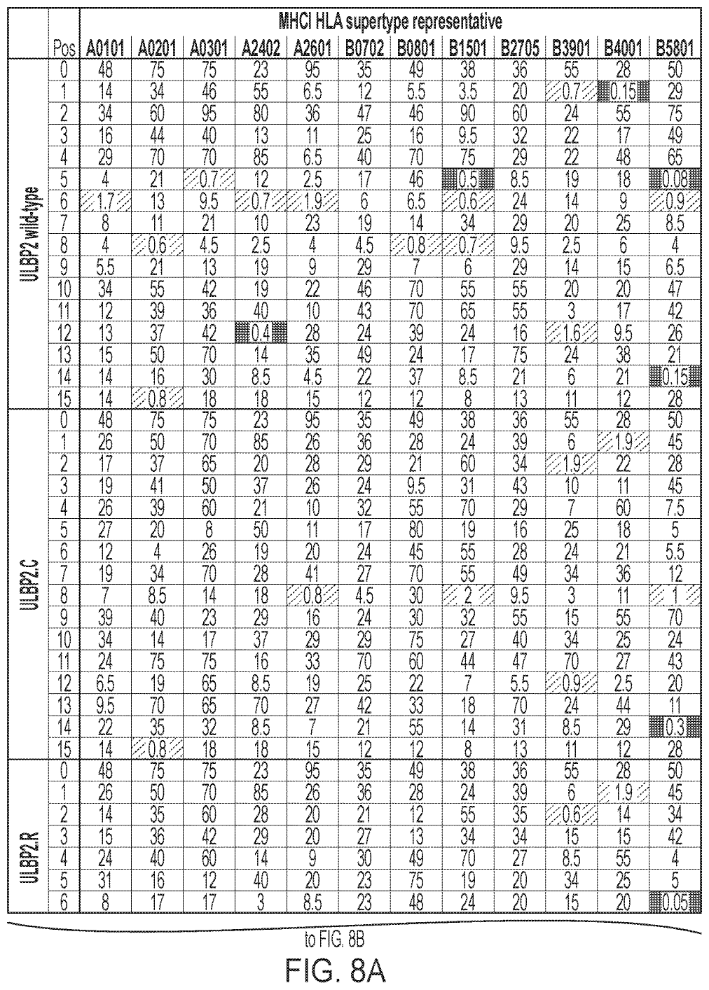

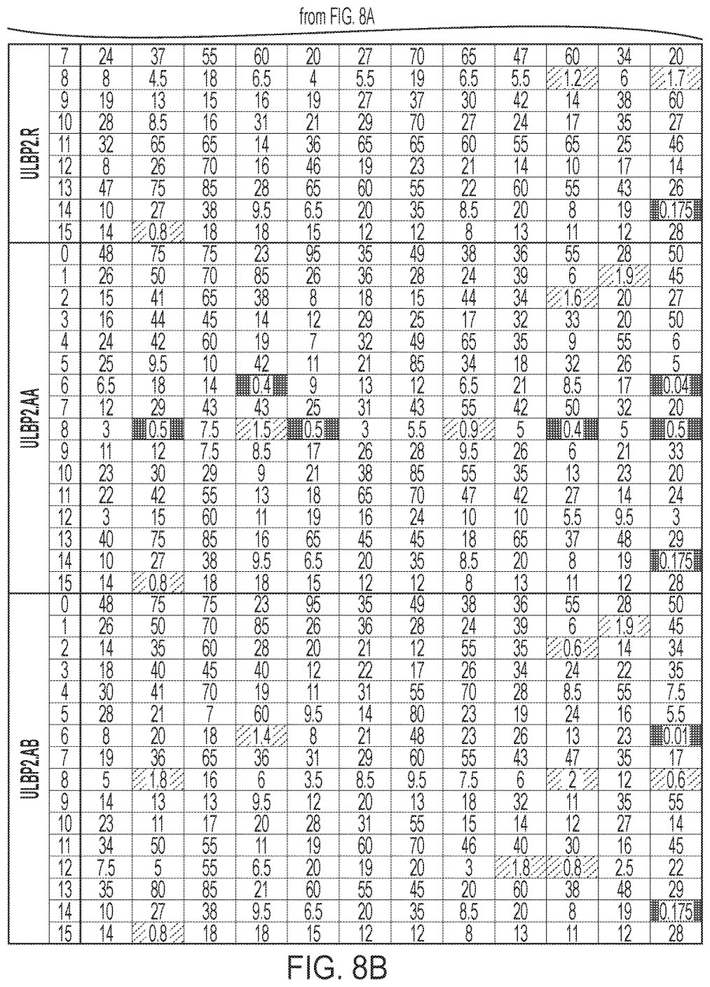

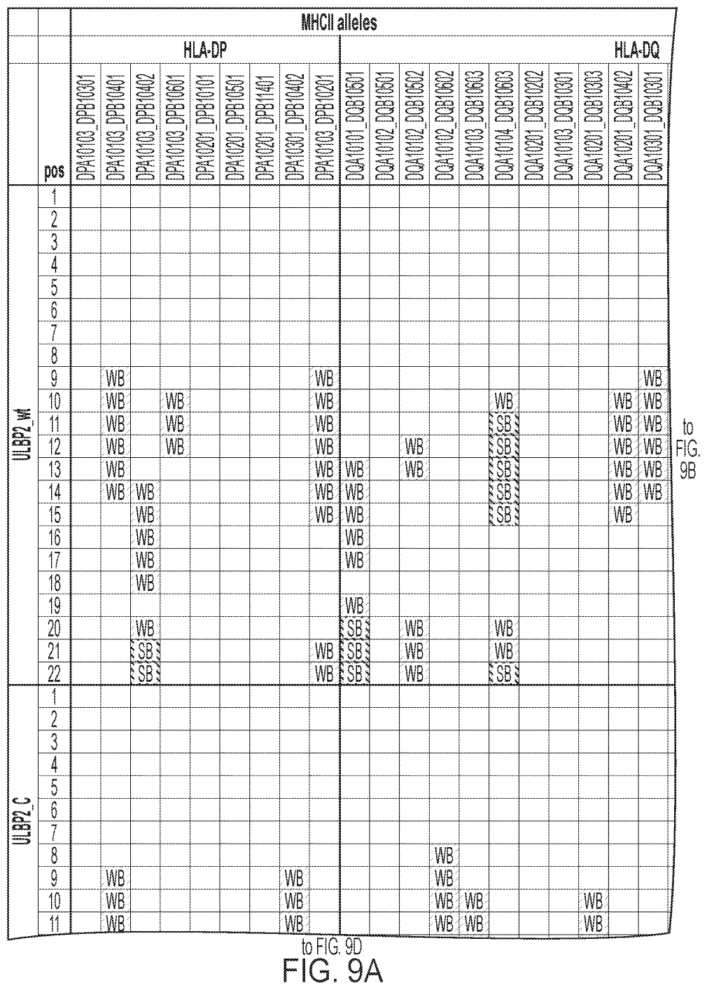

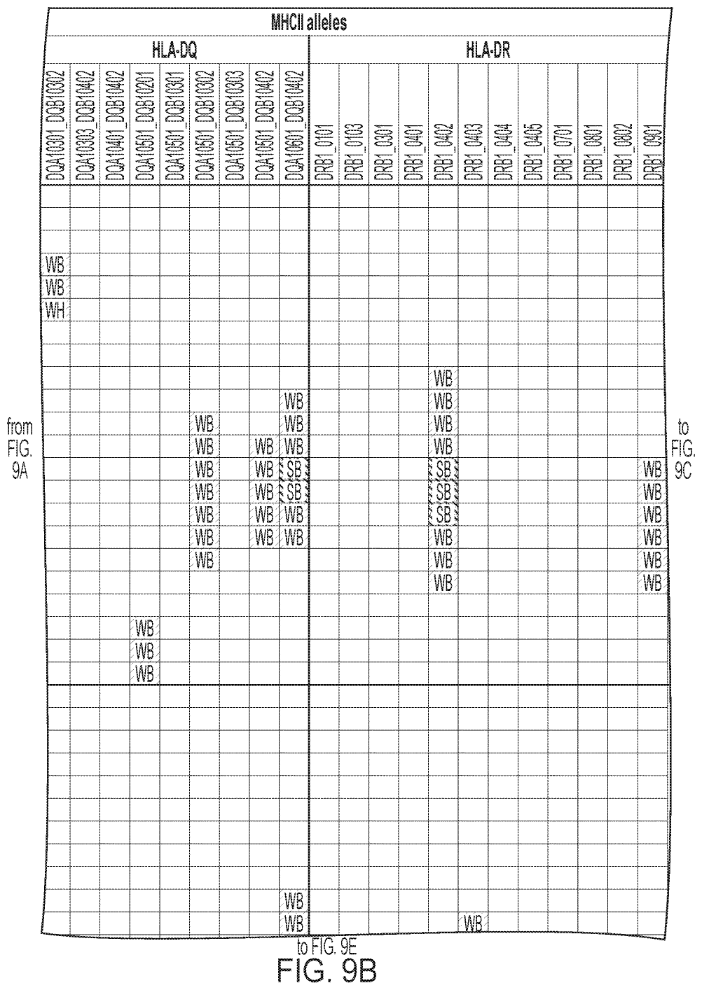

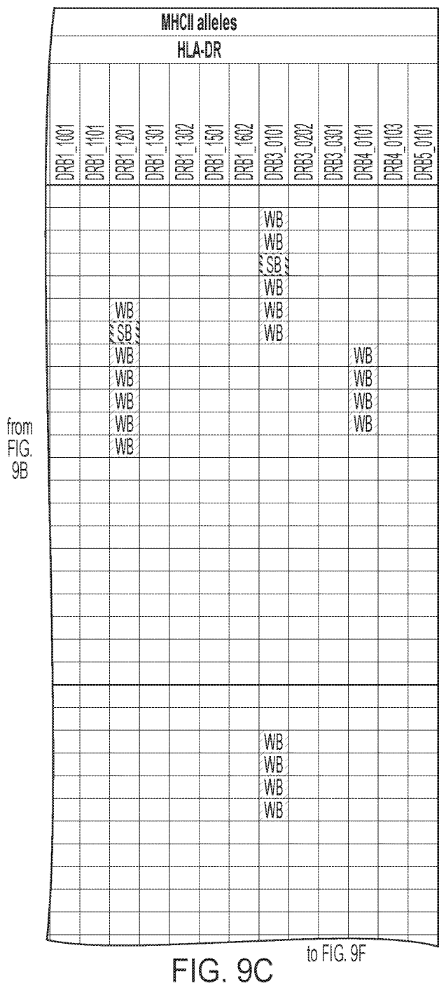

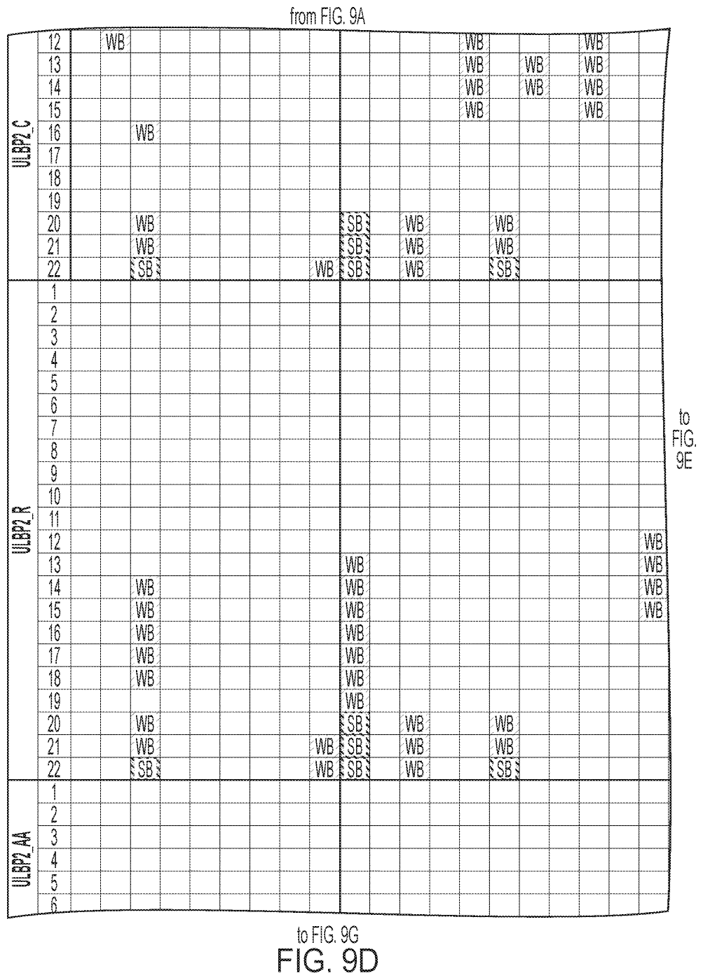

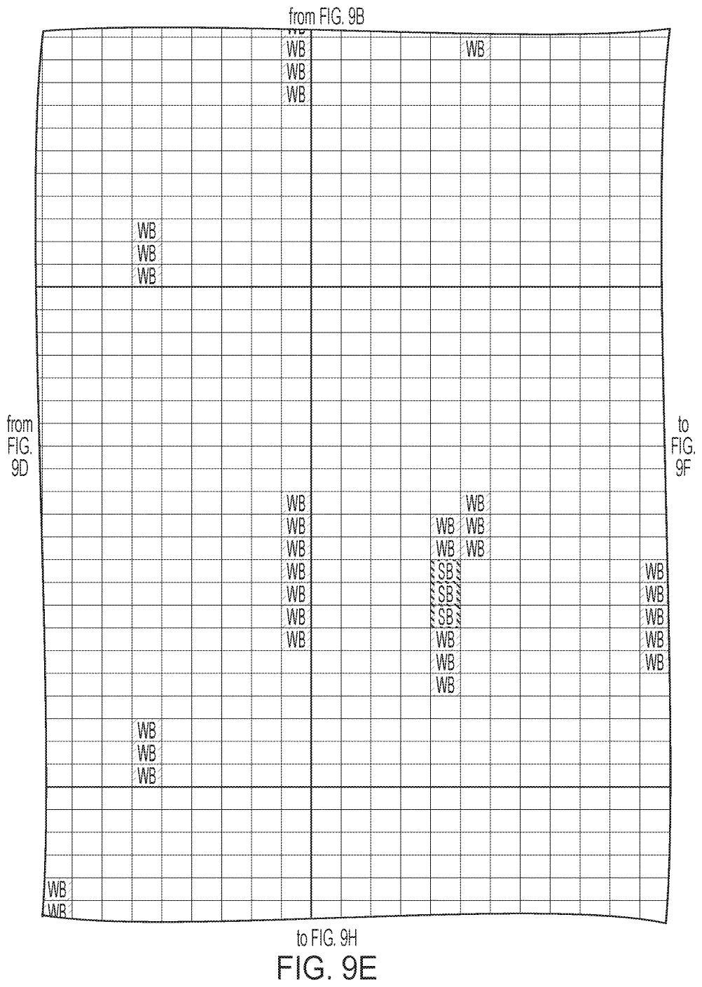

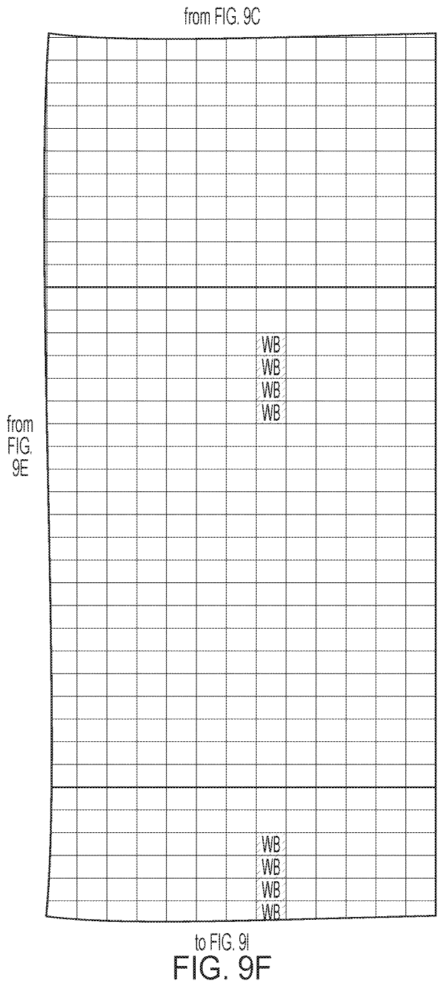

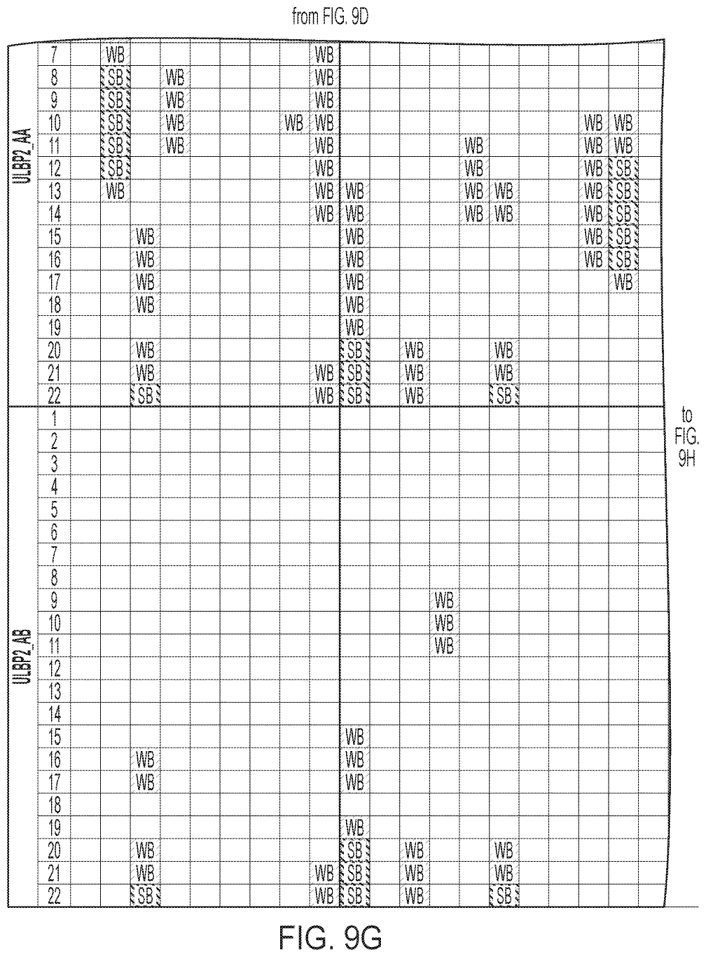

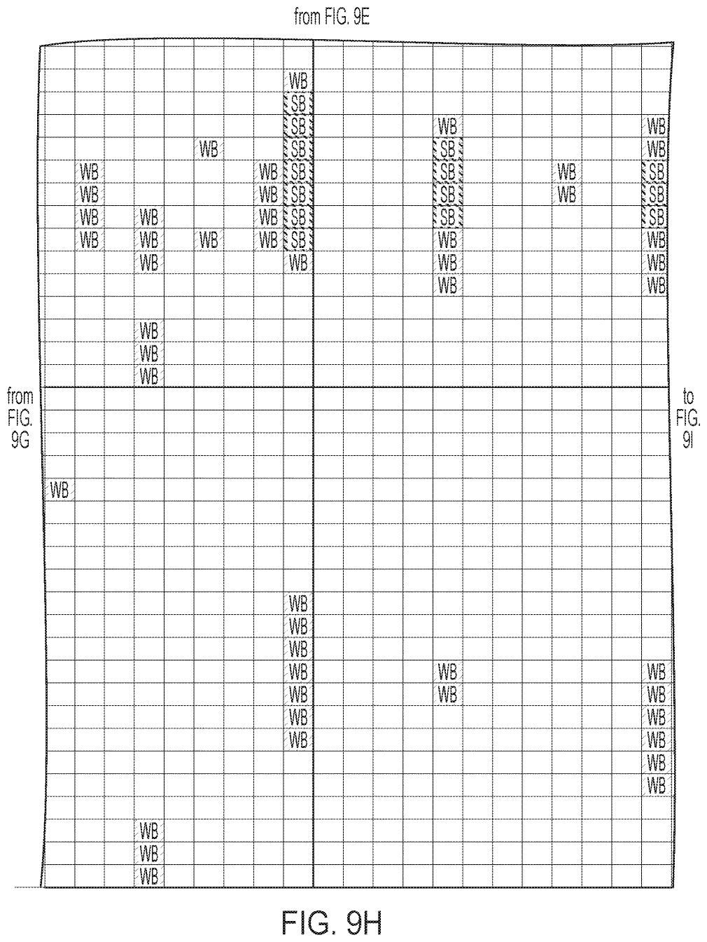

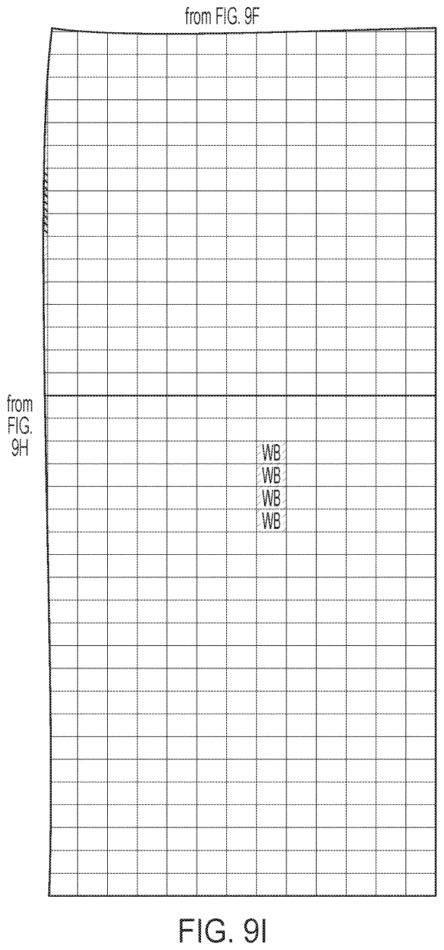

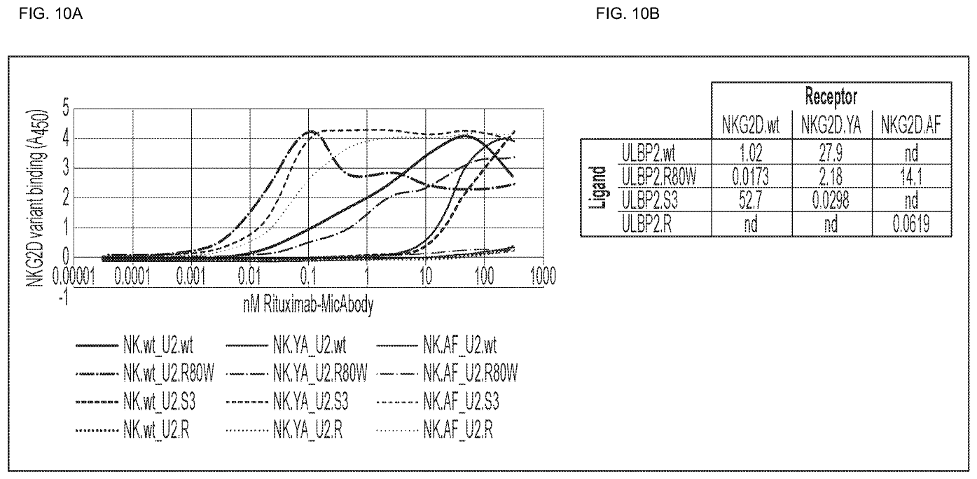

In order to determine whether a non-natural α1-α2 domain with selective binding to NKG2D.YA (ULBP2. S3, SEQ ID NO: 127) and the non-natural α1-α2 domains with selective binding to NKG2D.AF could discriminate between these two non-natural receptor variants, titration ELISAs were performed. All 21 of the selected α1-α2 variants that bound NKG2D.AF were directly compared for binding to NKG2D.AF versus NKG2D.YA. Of these, four demonstrated the properties of inability to bind NKG2D.wt, strong affinity for NKG2D.AF, and greatly reduced (15-20 fold) or eliminated binding to NKG2D.YA relative to NKG2D.AF (FIG. 7). These four non-natural ULBP2 α1-α2 variants—ULBP2.C, ULBP2.R, ULBP2. AA, and ULBP2. AB (SEQ ID NOs: 111, 113, 115, and 117)—were also examined for alterations in predicted immunogenicity profile relative to the wild-type ULBP2 peptide sequence (SEQ ID NO: 4) using the NetMHC4.0 Server (for peptide-MHC class I binding querying against all the HLA supertype representatives with 9-mer peptide analysis; http://www.cbs.dtu.dk/services/NetMHC/) and NetMHCII 2.3 Server (for peptide-MHC class II binding querying against HLA-DR, HLA-DQ, HLA-DP haplotypes with 15-mer peptide analysis; http://www.cbs.dtu.dk/services/NetMHCII/), both algorithms which were developed by the Technical University of Denmark (http://www.bioinformatics.dtu.dk/; Andreatta M and Nielsen M, Gapped sequence alignment using artificial neural networks: application to the MHC class I system, 2016 Bioinformatics, 32:511, PMID: 26515819; Jensen K K, Andreatta M, Marcatili P, Buus S, Greenbaum J A, Yan Z, Sette A, Peters B, and Nielsen M, Improved methods for predicting peptide binding affinity to MHC class I molecules, 2018 Immunology, PMID: 29315598). The mutations incorporated into ULBP2.C, ULBP2.R, and ULBP2. AB did not increase predicted immunogenicity while that of ULPB2. AA was increased slightly for a few haplotypes (FIGS. 8 and 9). As a consequence of the specificity of ULBP2.R for NKG2D.AF and its lack of predictable immunogenicity, ULBP2.R was selected for further ELISA analysis to directly compare its binding behavior with ULBP2. S3 (the NKG2D.YA-selected, non-natural, orthogonal ligand), ULBP2.R80W (non-natural ligand with enhanced affinity for wild-type NKG2D), and wild-type ULBP2 (ULBP2.wt). Binding of the four rituximab-MicAbody reagents (SEQ ID NOs: 98 and 121, 98 and 129, 131 and 100, and 98 and 106 as heavy chain and light chain for ULBP2.R, ULBP2. S3, ULBP2.R80W, and ULBP2.wt, respectively) was assayed against wild-type NKG2D (NKG2D.wt) and the two inert, non-natural variants NKG2D.YA and NKG2D.AF (FIGS. 10A-10B). The data demonstrated that NKG2D.YA-selected variant ULBP2. S3 as a MicAbody bound with high affinity to NKG2D.YA but did not engage NKG2D.AF or natural NKG2D. Furthermore, the NKG2D.AF-selected variant ULBP2.R in MicAbody format bound with high affinity to NKG2D.AF but did not engage NKG2D.YA or natural NKG2D. These results demonstrated the tremendous potential of exploring the NKG2D-MIC ligand axis and for developing unique pairs of novel, selective non-natural NKG2D receptors and their respective, cognate non-natural MIC ligand binding partners.

### Example 6: The Targeting and Killing Activity of CAR-T Cells Expressing the Non-Natural NKG2D.AF Ectodomain are Controlled by Orthogonal α1-α2 Domains Fused to Heterologous Targeting Polypeptides

Means to selectively control CAR-T cell therapies are highly sought after to mitigate toxicity and improve efficacy against tumors (Gill and June, op cit). Previous attempts have been made to develop CARs using the ectodomain of CD16 which can then be engaged through the Fc domain of therapeutic monoclonal antibodies, allowing for antibody-based control of CAR-T targeting (Chang et al., op cit). However, CD16-based CAR-T cells can recognize nearly all endogenous antibody molecules in blood and tissues, and the therapeutic antibodies used to control these cells will encounter competition from endogenous CD16 receptors on NK cells, PMN's, monocytes and macrophages. Both of these features contribute problems of off-tumor toxicity and poor pharmacokinetics, respectively.

Natural NKG2D ligands are present on certain healthy tissues and many stressed tissues, creating an extreme risk for toxicity using current NKG2D CAR approaches (VanSeggelen et al. 2015). The Y152A non-natural NKG2D receptor specifically bound to non-natural α1-α2 domain NKG2D ligands constituting an example of a means by which the activity of a non-natural NKG2D CAR could be selectively controlled using bispecific proteins comprised of the invented non-natural α1-α2 domain of NKG2D ligands.

We engineered CAR-T cells with a Receptor comprised of a modified Y152A/Y199F (“AF”) ectodomain of NKG2D which lacks binding to all natural NKG2D ligands or previously described non-natural α1-α2 domains orthogonal and cognate to Y152A modified NKG2D (NKG2D.YA). The invented cognate non-natural α1-α2 domains bound with high affinity to the non-natural NKG2D.AF ectodomain and avoided binding to natural NKG2D ectodomains and to the NKG2D.YA ectodomain. Thus, engineered α1-α2 domains that exhibited strong selectivity for non-natural NKG2D.AF ectodomain over natural NKG2D and non-natural NKG2D.YA represent an ideal system for selective control of non-natural NKG2D CAR receptors, or any receptor or protein fused to non-natural NKG2D ectodomains that can be selectively engaged by the non-natural α1-α2 domains of the instant invention. The instant invention further enables single cells expressing two distinct CARs—one comprised of NKG2D.YA and the other of NKG2D.AF—each signaling with distinctly different intracellular domains. These distinct CARs would possess independent, dual controls of the cell's activities by extracellular exposure to the respective, cognate orthogonal MicAbody or another non-antibody fusion polypeptide.

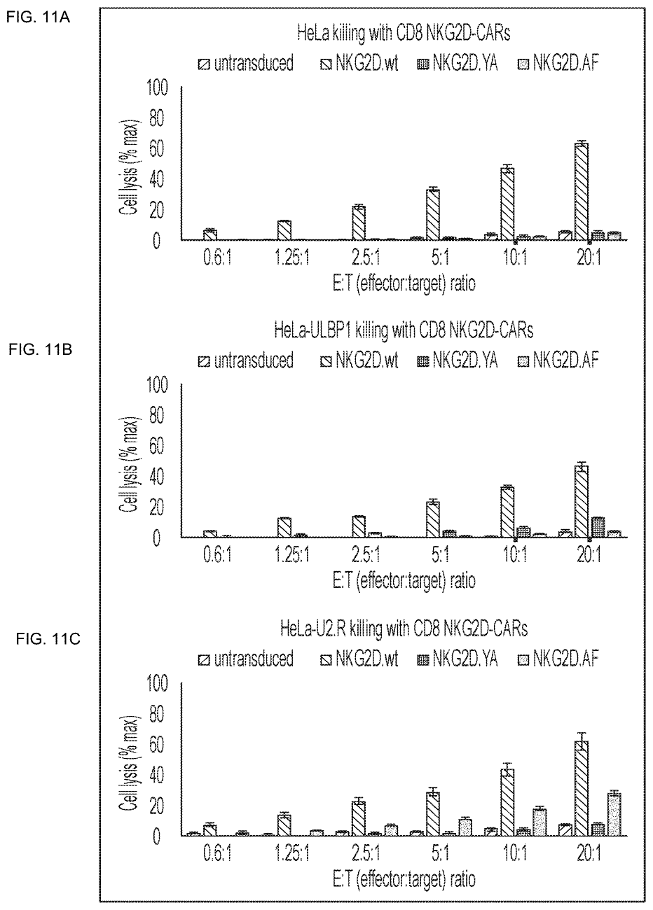

To demonstrate selective control of CAR-T cells constructed with a chimeric receptor deploying the non-natural NKG2D.AF ectodomain, we constructed CARs with either the natural NKG2D.wt (SEQ ID NO: 135), non-natural NKG2D.YA (SEQ ID NO: 137), or the non-natural NKG2D.AF (SEQ ID NO: 139) ectodomains based on previous work using 4-1BB/CD3-zeta CAR constructs (Campana U.S. Pat. No. 8,399,645) fusing the respective NKG2D ectodomains to the CD8 hinge region of CARs (SEQ ID NOs: 151, 153, 155). These constructs (SEQ ID NOs: 152, 154, 156) were cloned into a lentiviral vector and expressed in primary human CD8-positive T cells using lentiviral transduction. HeLa cells have constitutively upregulated levels of MIC ligands on their surface including MICA, MICB, ULBP3, and ULBP2/5/6 (the antibody used to ascertain this cannot distinguish between these three ULBPs; Human ULBP-2/5/6 Antibody, R&D Systems, Minneapolis, MN). HeLa cells were transfected to over-express either natural ULBP1 or the NKG2D.AF-selected variant ULBP2.R on their surface, and these cells were used as a target for in vitro killing assays. HeLa target cells were pre-loaded with calcein and exposed to NKG2D.wt-CAR, NKG2D.YA-CAR, or NKG2D.AF-CAR CD8 cells at increasing effector to target (E:T) ratios for five hours, after which the amount of calcein released into the supernatant was quantified and normalized to the total calcein released upon detergent treatment (FIGS. 11A-11C). Due to the elevated levels of MIC ligands naturally expressed on the surface of HeLa cells, the CD8 cells expressing natural NKG2D (NKG2D.wt) as the CAR engaged the HeLa cells via this over-expressed natural ligand and effected cytolysis. However, both the NKG2D.YA- and NKG2D.AF-CAR transduced CD8 cells demonstrated very little lysis of natural HeLa cells even at high E:T ratios, a level of activity that is on par with untransduced CD8 T cells. When ULBP1 is overexpressed on the surface of HeLa cells, only the NKG2D.wt-CAR CD8 T cells significantly lysed them. There is some additional killing at high E:T ratio with NKG2D.YA-CAR cells, but this is non-existent with NKG2D.AF-CAR cells showing that the double mutation Y152A/Y199F renders NKG2D even more inert than the single Y152A mutation. In HeLa cells over-expressing the NKG2D.AF-selective non-natural ULBP2.R, NKG2D.wt-CAR cells direct lysis (due to recognition of endogenous MIC ligands) while NKG2D.AF-CAR cells directed significant levels of lysis consistent with engagement of the receptor and its selective ligand.

In order to demonstrate that lysis of either NKG2D.YA- or NKG2D.AF-CAR cells could only be directed by the appropriate, cognate targeting MicAbody, Ramos cells were used as a target for cytolysis in combination with rituximab-based MicAbodies linked to either non-natural ULBP2. S3 or ULBP2.R orthogonal ligands. As demonstrated in FIG. 12A, the rituximab-ULBP2. S3 MicAbody could direct the cell killing activity of NKG2D.YA-CAR CD8 cells but not NKG2D.AF-CAR cells, while the rituximab-ULBP2.R MicAbody could direct the activity of NKG2D.AF-CAR but not NKG2D.YA-CAR cells. This further demonstrates the selectivity of the two non-natural ULBP2 variants for their cognate non-natural NKG2D variants for which they were engineered as preferred partners. In order to demonstrate the specificity of the antibody portion of the MicAbody, in vitro killing assays were performed with NKG2D.AF-CAR CD8 T-cells that were pre-armed by incubation with either rituximab-ULBP2.R, trastuzumab-ULPB2.R (SEQ ID NOs: 95 and 133, heavy and light chain, respectively), or an equimolar combination of the two at a saturating total concentration of MicAbody. After unbound MicAbody was removed by washing, the CD8 cells were applied to either Ramos cells (expressing CD20, the target of rituximab) or to CT26-Her2 (a mouse cell line transfected to express human Her2) that had been pre-loaded with calcein. After a two hour incubation at two different E:T ratios, the amount of calcein released was quantified. As shown in FIG. 12B, when cells were pre-armed with rituximab-MicAbody, only Ramos cells were lysed while trastuzumab-MicAbody directed cytolytic activity only against CT26-Her2 cells. However, when NKG2D.AF-CAR CD8 cells were simultaneously pre-armed with both rituximab- and trastuzumab-ULBP2.R MicAbodies, both target cells lines were lysed demonstrating that these CAR cells—by virtue of the selective, privileged partnering that has been engineered between receptor and ligand—were readily multiplexed and thereby directed to engage different tumor targets simultaneously.

### Example 7: Orthogonal α1-α2 Domains as a Means of Selectively Delivering Cytokines to Non-Natural Engineered NKG2D-Expressing T Cells

Bispecific MicAbodies (FIG. 13B) utilizing the antigen targeting Fv domains of antibodies and the privileged interaction between orthogonal α1-α2 domains and engineered non-natural NKG2D.YA or NKG2D.AF (SEQ ID NOs: 137 and 139, respectively) can effectively direct the cytolytic capabilities of NKG2D.YA- or NKG2D.AF-CAR bearing T cells (SEQ ID NOs: 153 and 155, respectively) to eliminate antigen-expressing target cells. Additionally, the highly selective interaction between cognate orthogonal ligands and non-natural, modified (also called “engineered”) NKG2D (eNKG2D) can be utilized to selectively deliver molecules to eNKG2D-ectodomain (eNKG2D-ecd)—bearing cells. If the heterologous atom or molecule (payload/cargo) to which the orthogonal ligand is fused is inherently bioactive with potentially undesirable functions when delivered to a non-eNKG2D expressing cell, mutations in the bioactive molecule can be explored that reduce interactions with native receptors or targets. When fused with an orthogonal ligand, the final molecule would therefore be effectively inert for all biological function except when cells bearing cognate eNKG2D receptor are present. Only when the high affinity interaction between orthogonal ligand and eNKG2D receptor occurs is interaction between the affinity-reduced mutant of the bioactive molecule and its natural target or receptor encouraged and residual function of the bioactive molecule enabled. All orthogonal ligand fusions to entities for the purpose of directing them to eNKG2D-expressing cells are collectively referred to as MicAdaptors and can, in principle, take the form of direct protein fusions as a single polypeptide between the payload and the orthogonal ligand at either the N- or C-terminus with or without a linker or tag (e.g. His tag) for assays or purification (FIG. 13C). Additionally, if enhanced serum stability is desired the antibody Fc domain may be included either as just the CH2-CH3 domains or in the format of a complete antibody, depending upon the desired number of unique payloads, the valency of either orthogonal ligand or heterologous cargo, and general experimentally determined architecture that promotes the greatest functionality of all components (FIG. 13D).

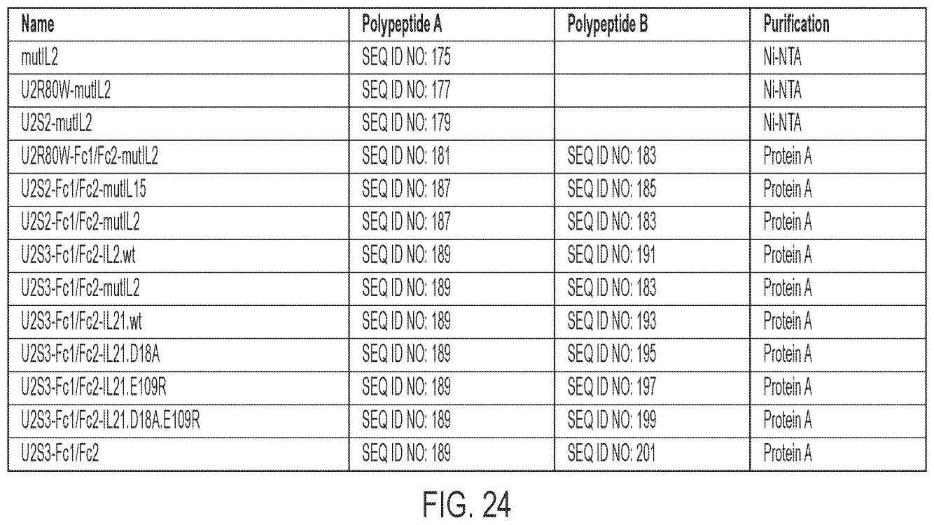

To determine whether cytokines can be selectively delivered to NKG2D.YA-CAR expressing cells (SEQ ID NO: 153), the cognate orthogonal ligand ULBP2. S3 (U2S3, SEQ ID NO: 127) was expressed fused to the N-terminus of Fc1 which has been altered to at two residues to express negatively charged aspartic acid residues at the interface of Fc homo-dimerization (SEQ ID NO: 189). A mutant form of human IL2 (mutIL2), containing two mutations R38A/F42K that significantly reduced the affinity of the cytokine to IL2R-alpha complex (K. M. Heaton, G. Ju, and E. A. Grimm, Human Interleukin 2 Analogues That Preferentially Bind the Intermediate-Affinity Interleukin 2 Receptor Lead to Reduced Secondary Cytokine Secretion: Implications for the Use of These Interleukin 2 Analogues in Cancer Immunotherapy, 1993 Cancer Res 53:2597, PMID: 8495422; K. Sauvé et al., Localization in Human Interleukin 2 of the Binding Site to the Alpha Chain (P55) of the Interleukin 2 Receptor, 1991 PNAS 88:4636, PMID: 2052547), was fused to the C-terminus of Fc2 which had been altered at two residues to express positively charged lysine residues at the interface of Fc homo-dimerization (SEQ ID NO: 183). Both were cloned independently into the mammalian expression vector pD2610-V12 (ATUM, Newark, CA), co-transfected into Expi293™ cells (ThermoFisher Scientific, Waltham, MA) according to the manufacturer's protocol, and purified using standard protein-A affinity chromatography (cat. no. 20334, Pierce Biotechnology, Rockford, IL). Purified material was fractionated by size-exclusion chromatography (SEC) on Akta Pur Superdex columns. The negatively charged residues on Fc1 and the positive charges on Fc2 provided an electrostatic steering effect (Kannan Gunasekaran et al., Enhancing Antibody Fc Heterodimer Formation through Electrostatic Steering Effects: Applications to Bispecific Molecules and Monovalent IgG, 2010 J Biol Chem 285:19637, PMID: 20400508) that promoted heterodimeric assembly of a molecule that was mono-valent for the orthogonal U2S3 ligand and mutIL2 (FIG. 13D, antibody (a)). Additionally, MicAdaptors comprised of other combinations of ULBP2 orthogonal ligands with cytokine fusions were explored—either as heterodimeric Fc1/Fc2 fusions or as a single polypeptide where both components were directly fused—and their DNAs similarly cloned, co-expressed, and purified as detailed in FIG. 24 and as described above. Recombinant human IL2 (rhIL2, Peprotech) and recombinant human IL15 (rhIL15, Peprotech) were included as controls in assays as needed.

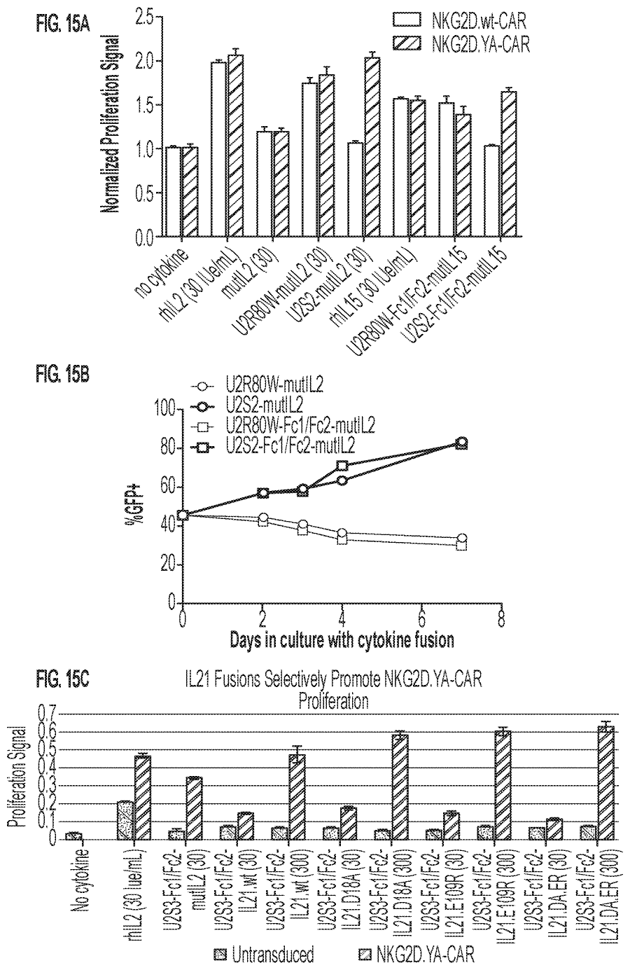

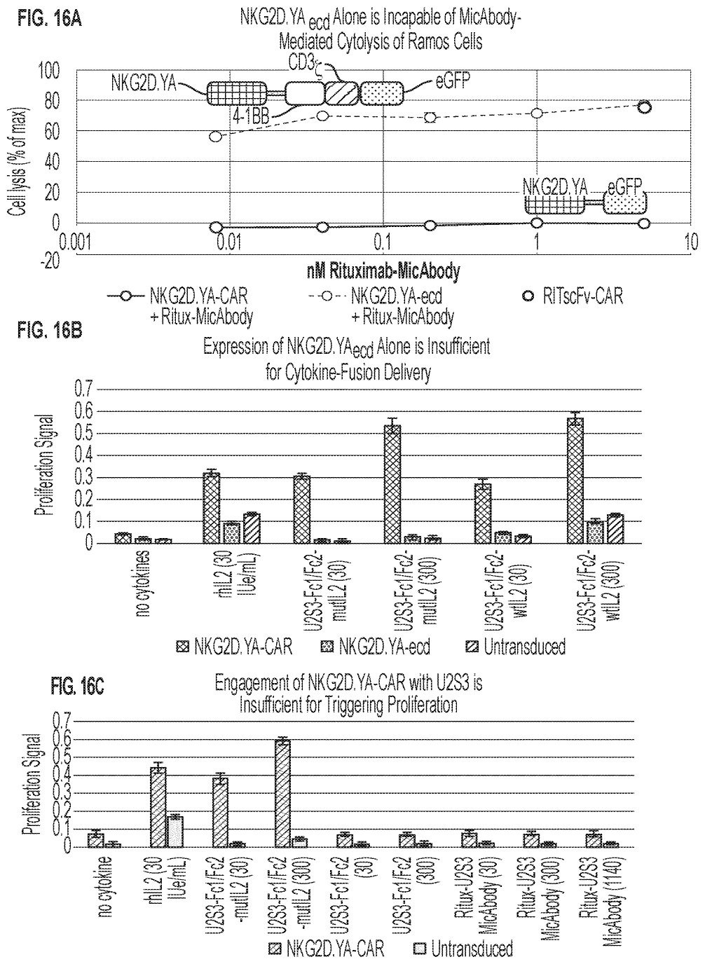

CD8 human T cells were transduced to express either the NKG2D.wt-CAR construct (SEQ ID NO: 151) or the NKG2D.YA-CAR construct (SEQ ID NO: 153) and exposed to 30 IUe/mL of control cytokine or various MicAdaptors for three days and the level of cell proliferation quantified with the WST-1 Cell Proliferation Reagent (Millipore Sigma). Control rhIL2 promoted proliferation of both CAR-expressing cell while mutIL2 alone did not as would be expected from reduced ability to engage IL2R-alpha and therefore reduced ability to signal through IL2R-beta/gamma-C(FIG. 15A). When the MicAdaptor was comprised of the non-selective U2R80W ligand (U2R80W-mutIL2, SEQ ID NO: 177), which engaged with high affinity to both wild-type and the modified NKG2D.YA receptors, cells expressing either of the CARs responded by proliferating. However, when the mutIL2 was fused to an orthogonal ligand, such as ULBP2. S2 (SEQ ID NO: 179) that selectively engages only NKG2D.YA, the MicAdaptor, U2S2-mutIL2) that only engages with NKG2D.YA, only NKG2D.YA-CAR cells responded by proliferating. Similar results were obtained when the cytokine fused to U2S2 was a mutant version of IL15 with a V49D mutation (Bernard, J. et al., Identification of an Interleukin-15c Receptor-binding Site on Human Interleukin-15, 2004 Journal of Biological Chemistry, 279:24313, PMID: 15039446) that reduced engagement with IL15R-alpha (FIG. 15A). When NKG2D.YA-CAR-T cells were co-cultured for seven days with these reagents, the % GFP+ (indicative of the percentage of CAR-expressing cells present as compared to the untransduced, GFP-negative cells) increased but only when the orthogonal ULBP2. S2 ligand (U2S2-mutIL2 or U2S2-Fc1/Fc2-mutIL2, FIG. 24 for SEQ ID NOs) was present and not with the non-selective U2R80W variant (which bound natural wild-type NKG2D receptor constitutively present on human CD8 cells) (FIG. 15B). IL21 was also explored as either a wild-type fusion (IL21.wt), with single mutations that affected IL21R-alpha binding (D18A or E109R), or a version with both mutations included (D18A/E109R) (Kang, L. et al., Rational Design of Interleukin-21 Antagonist through Selective Elimination of the γC Binding Epitope, 2010 Journal of Biological Chemistry, 285:12223, PMID: 20167599). In a comparison between untransduced CD8 cells or NKG2D.YA-CAR-expressing cells, only NKG2D.YA-bearing cells responded to all versions of IL21 fusions (see FIG. 24 for SEQ ID NOs), including to the IL21.wt fusion (FIG. 15C). Interestingly, while both untransduced and NKG2D.YA-CAR cells expanded in response to rhIL2 as compared to the no cytokine control, the amount of proliferation was higher with NKG2D.YA-CAR cells. The proliferative response of NKG2D.YA-CAR expressing cells was independent of a generalized binding or engagement of the NKG2D.YA domain since a U2S3-Fc1/Fc2 (heterodimeric Fc molecule with a single U2S3 domain and no attached cytokines or cytokine mutants) and the ritixumab-MicAbody (bivalent for the U2S3 domain but lacking any cytokine component, FIG. 13B, MicAbody (b)) did not induce proliferation over the course of three days of culture, even at IUe/mL concentrations much higher than with the rhIL2 control (FIG. 16C).

### Example 8: Presence of Intracellular Costimulatory Domains, Either in Cis or Trans, Promotes the Responsiveness of Non-Natural, Modified NKG2D-Bearing Cells to Cytokines and Cytokine MicAdaptors

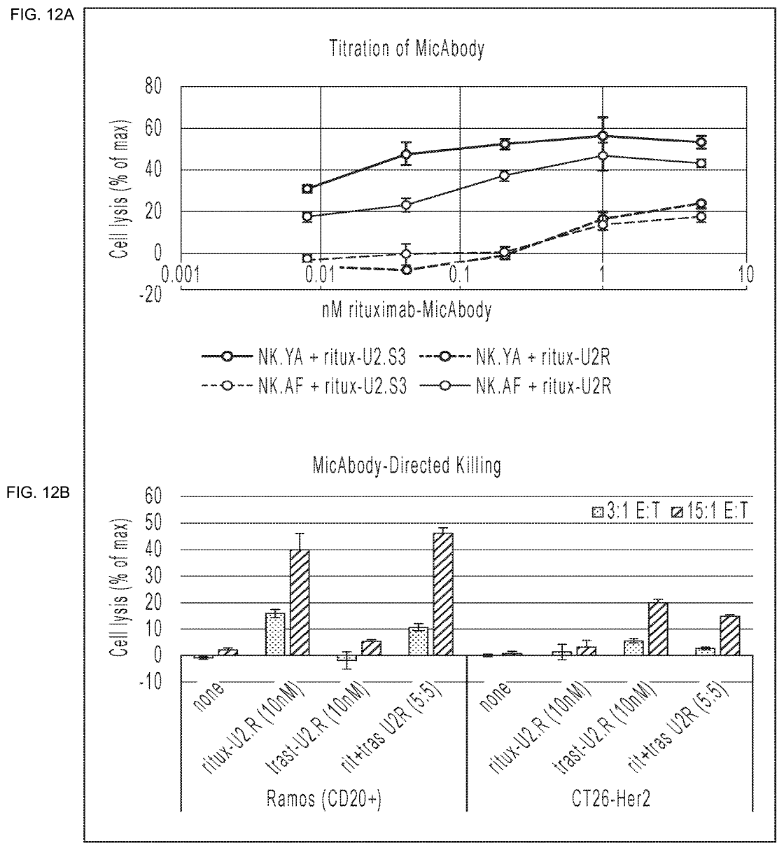

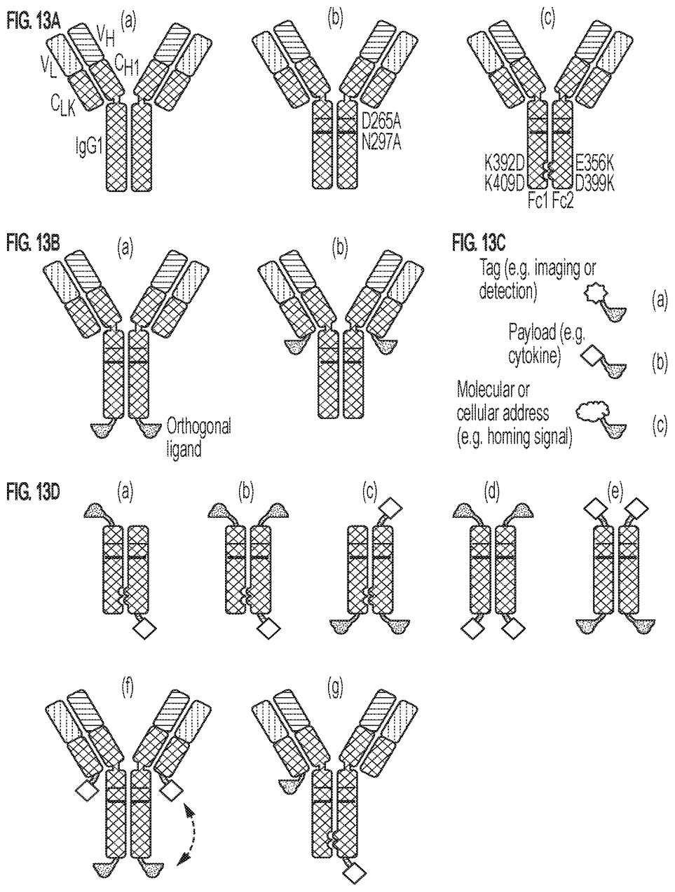

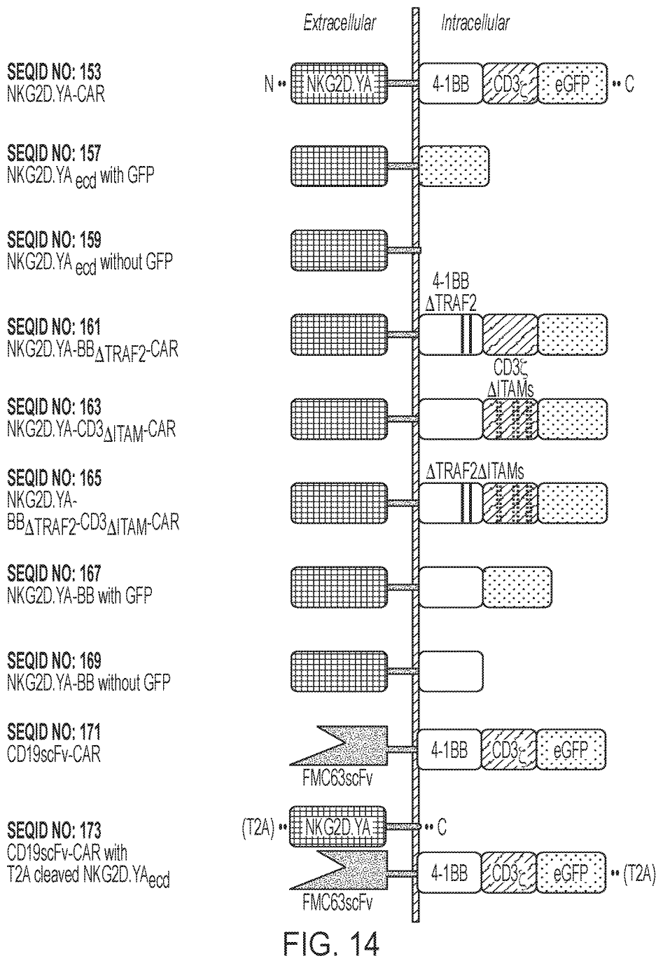

Upon demonstration that the modified NKG2D.YA domain, in the context of a chimeric antigen receptor construct, did in fact serve as a highly selective docking site for delivery of heterologous cargo attached to an orthogonal ligand, we sought to determine if the NKG2D.YA receptor was not only necessary but also sufficient for targeted delivery of cytokines that could then act on the receiving cell. The NKG2D.YA extracellular domain (NKG2D.YA-ecd) was expressed as a transmembrane domain stripped of all intracellular components with the exception of the retention of an intracellular eGFP tag (FIG. 14, SEQ ID NO: 157). CD8 cells were transduced to express this “silent CAR” and demonstrated to be unable to direct killing of Ramos target cells in the presence of a rituximab-ULBP2. S3 MicAbody (SEQ ID NOs: 98 and 129) as would be expected without costimulatory domains (FIG. 16A). Importantly, cells expressing this completely inert or silent CAR did not proliferate when exposed to U2S3-Fc1/Fc2-mutIL2 (SEQ ID NOs: 189 and 193), but responded at a level comparable to untransduced cells (FIG. 16B). This observation—in addition to (a) the consistent observation of greater levels of NKG2D.YA-CAR proliferation with rhIL2 compared to untransduced cells (FIGS. 15C, 16B, 16C) and (b) the observation that only NKG2D.YA-CAR cells responded to all forms of IL21-MicAdaptor including IL21.wt—led us to speculate that the intracellular domains present in the NKG2D.YA-CAR construct enhanced responsiveness to these cytokines and cytokine MicAdaptors.

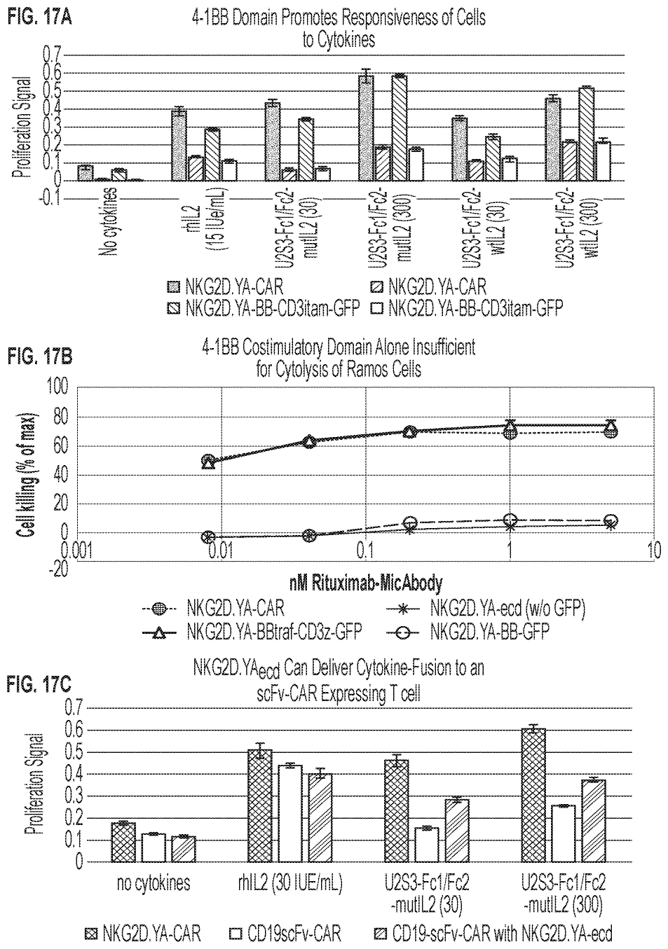

To examine this, a series of CAR constructs were generated where the signaling motifs of the intracellular domains of the CAR were mutated—either the two TRAF2 consensus-binding sites of 4-1BB (SEQ ID NO: 161), the three pairs of ITAM motifs in CD3-zeta (SEQ ID NO: 163), or combined 4-1BB/CD3-zeta mutants (SEQ ID NO: 165). These constructs (FIG. 14) were transduced into CD8 cells, co-incubated with the indicated cytokine reagents and proliferation quantified after three days (FIG. 17A). NKG2D.YA-BB-CD3_{ΔITAM}-GFP (SEQ ID NO: 163) retained a proliferative response on par with NKG2D.YA-CAR under all conditions tested, thereby demonstrated that the CD3-zeta domain (SEQ ID NO: 145) is dispensable for responsiveness to both cytokines and cytokine-MicAdaptors in the context of a CAR. However, NKG2D.YA-BB_{ΔTRAF2}-CD3zeta-GFP receptor-expressing cells (SEQ ID NO: 161) had significantly reduced responsiveness to all cytokines and cytokine-MicAdaptors as did the NKG2D.YA-BB_{ΔTRAF2}-CD3_{ΔITAM}-GFP (SEQ ID NO: 165) receptor bearing mutations in both intracellular domains. This indicated that the costimulatory 4-1BB (SEQ ID NO: 143) played a role in responsiveness of NKG2D.YA-CAR cells to both cytokines and cytokine-MicAdaptors and that a silent CAR expressed on a cell surface needed to also have the an intracellular costimulatory domain present to promote cytokine-MicAdaptor responsiveness. The inability of an NKG2D.YA-BB silent CAR (SEQ ID NO: 169, FIG. 14) to direct MicAbody-mediated cell killing (FIG. 17B) demonstrated that this CAR architecture that lacked CD3-zeta was suitable as the silent docking site for MicAdaptors and enabled cytokine-MicAdaptors signaling but did not function to mediate cytolysis of a MicAbody targeted cell.

To further explore how the 4-1BB domain contributes to cytokine and cytokine-MicAdaptor responsiveness, a construct was generated where a CD19scFv-CAR (based on FMC63 Fv's), containing the full complement of functional 4-1BB and CD3-zeta domains (SEQ ID NO: 173). The CD19scFv-CAR was co-expressed with the NKG2D.YA-ecd (FIG. 14). Both components were expressed as a single polypeptide with a T2A self-cleaving peptide motif separating the upstream CD19scFv-CAR construct from the downstream NKG2D.YA-ecd which possessed an independent GMCSFR alpha chain signal sequence, CD8a hinge, and CD8a transmembrane domain. This construct was transduced into CD8 T-cells, and the co-expression of the CD19scFv-CAR and the NKG2D.YA-ecd on the surface was verified by flow cytometry examining GFP signal and phycoerythrin-conjugated MicAbody staining, respectively. These cells were incubated for three days with cytokines and cytokine-MicAdaptors and proliferation quantified by WST assay. The NKG2D.YA-CAR responded to both rhIL2 and U2S3-Fc1/Fc2-mutIL2 as expected although control cells harboring only the CD19scFv-CAR (SEQ ID NO: 171) expanded with rhIL2 and to a lesser degree to the highest concentration (300 IUe/mL) of U2S3-Fc1/Fc2-mutIL2 (FIG. 17C). Coexpression of the NKG2D.YA-ecd on CD19scFv-CAR expressing cells (SEQ ID NO: 173) exhibited greater proliferative response to the U2S3-Fc1/Fc2-mutIL2 cytokine-MicAdaptor than cells expressing only CD19scFv-CAR (SEQ ID NO: 171). In this context, the 4-1BB domain was constitutively provided in trans to NKG2D.YA-ecd. These data demonstrated that responsiveness of NKG2D.YA-ecd expressing cells to cytokines and cytokine-MicAdaptors was promoted by the 4-1BB domain either in cis or trans and that the NKG2D.YA-ecd domain can be coexpressed with a costimulatory 4-1BB-containing CAR cell to provide added versatile functionality to engineered cells of adoptive cell therapy strategies. This functionality is not only limited to engagement of surface receptors upon ligand/silent CAR engagement, but could be extended to intracellular delivery by incorporating cytoplasmic sequence motifs (K. N. Pandey, Functional roles of short sequence motifs in the endocytosis of membrane receptors, 2009 Front Biosci, 14:5339, PMID: 19482617) that promote the turnover of non-natural NKG2D variants such that any bound MicAdaptor would be co-internalized and the heterologous cargo delivered intracellularly.

1.-7. (canceled)

8. A non-natural, modified NKG2D receptor attached to a mammalian cell wherein a costimulatory domain is attached intracellularly to the modified NKG2D receptor without the presence of an active CD3-zeta domain, wherein the receptor binds non-natural, modified α1-α2 domains of NKG2D ligands but not natural ligands of NKG2D.

9. The non-natural, modified α1-α2 domains of claim 8 to which a heterologous atom or molecule is attached wherein the heterologous atom or molecule when delivered to the mammalian cell by a modified α1-α2 domain, can be internalized by the mammalian cell wherein the internalized heterologous atom or molecule either bound or unbound to the modified α1-α2 domain affects the mammalian cell.

10. The non-natural, modified α1-α2 domains of claim 8 to which a heterologous atom or molecule is attached wherein the heterologous atom or molecule provides to the mammalian cell a targeting or homing function that promotes the delivery of the mammalian cell to a target-bearing cell or target-bearing surface without directly leading to the activation of the mammalian cell when it reaches its targeted surface or environment.

11. The NKG2D-attached costimulatory domain of claim 8 wherein the costimulatory domain enhances the function of an effector molecule introduced to the mammalian cell wherein the effector molecule is not attached to a modified α1-α2 domain.


SEQUENCE LISTING (202 sequences)

SEQ ID NO: 1 - protein, 183 aa, synthetic construct
  REGION 1..183: note = Synthetic peptide MICA alpha1-alpha2 domain
AAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRT

SEQ ID NO: 2 - protein, 182 aa, synthetic construct
  REGION 1..182: note = Synthetic peptide MICB alpha1-alpha2 domain
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRT

SEQ ID NO: 3 - protein, 187 aa, synthetic construct
  REGION 1..187: note = Synthetic peptide ULBP1 alpha1-alpha2 domain
AAEPHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPMV

SEQ ID NO: 4 - protein, 189 aa, synthetic construct
  REGION 1..189: note = Synthetic peptide ULBP2 alpha1-alpha2 domain
AAEPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPPMV

SEQ ID NO: 5 - protein, 187 aa, synthetic construct
  REGION 1..187: note = Synthetic peptide ULBP3 alpha1-alpha2 domain
AAEPHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPMV

SEQ ID NO: 6 - protein, 187 aa, synthetic construct
  REGION 1..187: note = Synthetic peptide ULBP4 alpha1-alpha2 domain
AAEPHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPPMV

SEQ ID NO: 7 - protein, 198 aa, synthetic construct
  REGION 1..198: note = Synthetic peptide ULBP5 alpha1-alpha2 domain
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 8 - protein, 189 aa, synthetic construct
  REGION 1..189: note = Synthetic peptide ULBP6 alpha1-alpha2 domain
AAEPHSLCYDITVIPKFRPGPRWCAVQGQVDEKTFLHYDCGNKTVTPVSPLGKKLNVTTAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPPMV

SEQ ID NO: 9 - protein, 161 aa, synthetic construct
  REGION 1..161: note = Synthetic peptide OMCP alpha1-alpha2 domain
AAAEPHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRRTVPPMV

SEQ ID NO: 10 - DNA, 543 bp, synthetic construct
  misc_feature 1..543: note = Synthetic polynucleotide, encoding MICA alpha1-alpha2 domain
  source 1..543: mol_type = other DNA
gagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaaca

SEQ ID NO: 11 - DNA, 561 bp, synthetic construct
  misc_feature 1..561: note = Synthetic polynucleotide, encoding ULBP1 alpha1-alpha2 domain
  source 1..561: mol_type = other DNA
gctgctgagccccactgtctctgctacgactttattataactcctaagtcaagaccagagcctcagtggtgcgaagtacaaggtttggttgacgaaaggcctttccttcactacgattgtgtgaaccataaggcaaaggctttcgccagcctgggtaagaaggtaaacgttactaagacgtgggaggagcagacggaaaccctccgtgatgtggttgactttcttaagggtcagctcctcgatatccaagtggagaatttaatccctatcgaaccgctcactctgcaggccagaatgtcatgcgaacatgaagcacacggtcatggaagaggtagttggcaatttttatttaacggtcaaaaattcctgctgttcgactcaaacaaccgcaaatggactgcgctgcaccctggagctaagaagatgactgaaaaatgggagaagaacagagacgttaccatgttcttccagaagatttccctgggagattgtaagatgtggttagaggagttcttaatgtactgggaacagatgctggaccccacaaaaccccccatggtg

SEQ ID NO: 12 - DNA, 567 bp, synthetic construct
  misc_feature 1..567: note = Synthetic polynucleotide, encoding ULBP2 alpha1-alpha2 domain
  source 1..567: mol_type = other DNA
gctgctgagccccatagtctgtgttacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgcgcgacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcggctggtgccccccccatggtg

SEQ ID NO: 13 - DNA, 561 bp, synthetic construct
  misc_feature 1..561: note = Synthetic polynucleotide, encoding ULBP3 alpha1-alpha2 domain
  source 1..561: mol_type = other DNA
gctgctgagccccacagcttgtggtacaacttcaccattatccacttgccgagacatggccagcagtggtgcgaagtgcaatcgcaagtcgaccaaaaaaacttcttatcatacgactgcggcagcgataaggtcttatctatgggtcatttggaggaacagctctacgcgaccgacgcctggggtaaacagctcgagatgctccgtgaggttggacagaggctgagactggaactggctgacactgagctggaagatttcacacctagtggtccactcacattgcaagtacgcatgagctgcgagtgtgaggccgatggatacattaggggcagctggcagtttagcttcgacggaaggaaattcctgctcttcgacagtaacaataggaagtggactgttgtgcatgctggtgcgcgcagaatgaaggaaaagtgggagaaagatagcggcctgacgaccttcttcaagatggtgtctatgcgtgactgtaagagctggctcagagatttcctcatgcatcgcaagaagaggttagaacctaccgctccccccatggtg

SEQ ID NO: 14 - DNA, 561 bp, synthetic construct
  misc_feature 1..561: note = Synthetic polynucleotide, encoding ULBP4 alpha1-alpha2 domain
  source 1..561: mol_type = other DNA
gctgctgagccccactctctttgcttcaacttcaccattaaatccctgagcaggcctggtcagccgtggtgtgaggcgcaggtctttcttaacaagaatctcttcctccaatacaactctgataacaacatggtaaagccactgggtctcctgggtaaaaaagtctatgctacgagcacttggggagaactcacccagactcttggcgaggtaggaagagacctgcgcatgctcctctgcgatataaagccccaaattaagaccagtgatccgtccactttacaagtcgaaatgttctgccaaagggaggctgaacgctgcaccggagcctcttggcagttcgcgaccaatggcgaaaagtccctcttgttcgatgccatgaatatgacctggaccgtgatcaatcatgaggcctctaagatcaaggagacgtggaaaaaggaccgcggccttgaaaagtactttaggaagttgtctaaaggagactgcgaccattggttacgcgagttcctcggccattgggaagcgatgcccgagccaacggttagcccccccatggtg

SEQ ID NO: 15 - DNA, 567 bp, synthetic construct
  misc_feature 1..567: note = Synthetic polynucleotide, encoding ULBP6 alpha1-alpha2 domain
  source 1..567: mol_type = other DNA
gctgctgagccccactccttatgctatgatatcaccgtgattccaaagttccgaccaggaccccgatggtgcgccgtacagggacaggtcgacgaaaagacttttttacattacgactgcggtaacaagacagtcacaccggtaagtcctttgggaaaaaagttaaacgtaaccactgcttggaaggcccagaaccccgtccttcgagaagtagtggatattttgactgaacagctgcttgacatccagctggaaaactacacacccaaagagcccctgactcttcaagcgcgtatgtcgtgtgagcaaaaggccgaaggacacagctccggatcctggcagttcagtatcgacggtcagaccttcctcctcttcgattcagaaaagcgcatgtggactactgtgcaccccggcgctcgtaagatgaaggaaaagtgggagaatgataaggacgttgccatgagttttcattacattagtatgggagattgcatcggttggctggaagacttcctgatgggtatggatagtacccttgaacctagtgccggagctccccccatggtg

SEQ ID NO: 16 - DNA, 483 bp, synthetic construct
  misc_feature 1..483: note = Synthetic polynucleotide, encoding OMCP alpha1-alpha2 domain
  source 1..483: mol_type = other DNA
gctgctgctgagccccacaagcttgcgttcaacttcaatctggaaataaacggttcagatacccattcaaccgtggacgtttatttagacgattcgcagataatcacctttgacggcaaggacatccgcccaactatcccgttcatgataggtgacgaaatcttccttcctttttataagaatgtgttctctgagttcttcagtttgttccgccgcgtccctacctcaaccccctacgaagacttgacttatttctatgaatgcgactacaccgacaacaaatctacattcgatcaattctacctgtacaacggtgaagagtacaccgtgaagactcaagaggctactaacaagaacatgtggctgaccacttccgagttcagactgaagaagtggttcgacggcgaggactgtatcatgcaccttagaagtttagtgaggaaaatggaagatagcaagagaagaacagtgccccccatggtg

SEQ ID NO: 17 - protein, 139 aa, synthetic construct
  REGION 1..139: note = Synthetic peptide natural NKG2D ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 18 - protein, 139 aa, synthetic construct
  REGION 1..139: note = Synthetic peptide non-natural NKG2D Y152A ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 19 - protein, 139 aa, synthetic construct
  REGION 1..139: note = Synthetic peptide non-natural NKG2D Y199A ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGAIENCSTPNTYICMQRTV

SEQ ID NO: 20 - protein, 139 aa, synthetic construct
  REGION 1..139: note = Synthetic peptide non-natural NKG2D Y152A/Y199A ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGAIENCSTPNTYICMQRTV

SEQ ID NO: 21 - protein, 139 aa, synthetic construct
  REGION 1..139: note = Synthetic peptide non-natural NKG2D Y199F eNKG2D1 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGFIENCSTPNTYICMQRTV

SEQ ID NO: 22 - protein, 139 aa, synthetic construct
  REGION 1..139: note = Synthetic peptide non-natural NKG2D Y152S eNKG2D2 ectodomain
FLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGYIENCSTPNTYICMQRTV

SEQ ID NO: 23 - protein, 139 aa, synthetic construct
  REGION 1..139: note = Synthetic peptide non-natural NKG2D Y152T eNKG2D3 ectodomain
FLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGYIENCSTPNTYICMQRTV

SEQ ID NO: 24 - protein, 139 aa, synthetic construct
  REGION 1..139: note = Synthetic peptide non-natural NKG2D Y152V eNKG2D4 ectodomain
FLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGYIENCSTPNTYICMQRTV

SEQ ID NO: 25 - protein, 139 aa, synthetic construct
  REGION 1..139: note = Synthetic peptide non-natural NKG2D Y152A/Y199F eNKG2D5 ectodomain
FLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 26 - protein, 139 aa, synthetic construct
  REGION 1..139: note = Synthetic peptide non-natural NKG2D Y152L/Y199F eNKG2D6 ectodomain
FLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGFIENCSTPNTYICMQRTV

SEQ ID NO: 27 - protein, 139 aa, synthetic construct
  REGION 1..139: note = Synthetic peptide non-natural NKG2D Y152S/Y199F eNKG2D7 ectodomain
FLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGFIENCSTPNTYICMQRTV

SEQ ID NO: 28 - protein, 139 aa, synthetic construct
  REGION 1..139: note = Synthetic peptide non-natural NKG2D Y152T/Y199F eNKG2D8 ectodomain
FLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGFIENCSTPNTYICMQRTV

SEQ ID NO: 29 - protein, 139 aa, synthetic construct
  REGION 1..139: note = Synthetic peptide non-natural NKG2D Y152V/Y199F eNKG2D9 ectodomain
FLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGFIENCSTPNTYICMQRTV

SEQ ID NO: 30 - protein, 139 aa, synthetic construct
  REGION 1..139: note = Synthetic peptide non-natural NKG2D Y199D eNKG2D10 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGDIENCSTPNTYICMQRTV

SEQ ID NO: 31 - protein, 139 aa, synthetic construct
  REGION 1..139: note = Synthetic peptide non-natural NKG2D Y199E eNKG2D11 ectodomain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGEIENCSTPNTYICMQRTV

SEQ ID NO: 32 - protein, 139 aa, synthetic construct
  REGION 1..139: note = Synthetic peptide non-natural NKG2D Y152D/Y199D eNKG2D12 ectodomain
FLNSLFNQEVQIPLTESYCGPCPKNWICYKNNCYQFFDESKNWYESQASCMSQNASLLKVYSKEDQDLLKLVKSDHWMGLVHIPTNGSWQWEDGSILSPNLLTIIEMQKGDCALYASSFKGDIENCSTPNTYICMQRTV

SEQ ID NO: 33 - protein, 139 aa, synthetic construct
  REGION 1..139: note = Synthetic peptide non-natural NKG2D Y152E/Y199E eNKG2D13 ectodomain
FLNSLFNQEVQIPLTESYCGPCPKNWICYKNNCYQFFDESKNWYESQASCMSQNASLLKVYSKEDQDLLKLVKSEHWMGLVHIPTNGSWQWEDGSILSPNLLTIIEMQKGDCALYASSFKGEIENCSTPNTYICMQRTV

SEQ ID NO: 34 - protein, 139 aa, synthetic construct
  REGION 1..139: note = Synthetic peptide non-natural NKG2D Y152L eNKG2D14 ectodomain
FLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGYIENCSTPNTYICMQRTV

SEQ ID NO: 35 - protein, 139 aa, synthetic construct
  REGION 1..139: note = Synthetic peptide non-natural NKG2D Y152F/Y199F eNKG2D15 ectodomain
FLNSLFNQEVQIPLTESYCGPCPKNWICYKNNCYQFFDESKNWYESQASCMSQNASLLKVYSKEDQDLLKLVKSFHWMGLVHIPTNGSWQWEDGSILSPNLLTIIEMQKGDCALYASSFKGFIENCSTPNTYICMQRTV

SEQ ID NO: 36 - protein, 23 aa, synthetic construct
  REGION 1..23: note = Synthetic peptide MHCI signal sequence
MGLGPVFLLLAGIFPFAPPGAAA

SEQ ID NO: 37 - DNA, 69 bp, synthetic construct
  misc_feature 1..69: note = Synthetic polynucleotide, encoding MHCI signal sequence
  source 1..69: mol_type = other DNA
atgggccttggcccagtgtttctgctgttggcaggcattttcccttttgctccgcccggcgccgcagcc

SEQ ID NO: 38 - protein, 237 aa, synthetic construct
  REGION 1..237: note = Synthetic peptide human IgG1 Fc with IEGR linker
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGR

SEQ ID NO: 39 - DNA, 711 bp, synthetic construct
  misc_feature 1..711: note = Synthetic polynucleotide, encoding human IgG1 Fc with IEGR linker
  source 1..711: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgc

SEQ ID NO: 40 - protein, 376 aa, synthetic construct
  REGION 1..376: note = Synthetic peptide human IgG1 Fc-NKG2D fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 41 - protein, 376 aa, synthetic construct
  REGION 1..376: note = Synthetic peptide human IgG1 Fc-NKG2D Y152A fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 42 - protein, 376 aa, synthetic construct
  REGION 1..376: note = Synthetic peptide human IgG1 Fc-NKG2D Y199A ectodomain
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGAIENCSTPNTYICMQRTV

SEQ ID NO: 43 - protein, 376 aa, synthetic construct
  REGION 1..376: note = Synthetic peptide human IgG1 Fc-NKG2D Y152A/Y199A ectodomain
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGAIENCSTPNTYICMQRTV

SEQ ID NO: 44 - protein, 376 aa, synthetic construct
  REGION 1..376: note = Synthetic peptide human IgG1 Fc-NKG2D Y199F eNKG2D1 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGFIENCSTPNTYICMQRTV

SEQ ID NO: 45 - protein, 376 aa, synthetic construct; identical to SEQ ID NO: 44
  REGION 1..376: note = Synthetic peptide human IgG1 Fc-NKG2D Y152S eNKG2D2 fusion

SEQ ID NO: 46 - protein, 376 aa, synthetic construct
  REGION 1..376: note = Synthetic peptide human IgG1 Fc-NKG2D Y152T eNKG2D3 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGYIENCSTPNTYICMQRTV

SEQ ID NO: 47 - protein, 376 aa, synthetic construct
  REGION 1..376: note = Synthetic peptide human IgG1 Fc-NKG2D Y152V eNKG2D4 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGYIENCSTPNTYICMQRTV

SEQ ID NO: 48 - protein, 376 aa, synthetic construct
  REGION 1..376: note = Synthetic peptide human IgG1 Fc-NKG2D Y152A/Y199F eNKG2D5 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 49 - protein, 376 aa, synthetic construct
  REGION 1..376: note = Synthetic peptide human IgG1 Fc-NKG2D Y152L/Y199F eNKG2D6 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGFIENCSTPNTYICMQRTV

SEQ ID NO: 50 - protein, 376 aa, synthetic construct
  REGION 1..376: note = Synthetic peptide human IgG1 Fc-NKG2D Y152S/Y199F eNKG2D7 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSSHWMGLVHIPTNGSWQWEDGSILSPNLLTIIEMQKGDCALYASSFKGFIENCSTPNTYICMQRTV

SEQ ID NO: 51 - protein, 376 aa, synthetic construct
  REGION 1..376: note = Synthetic peptide human IgG1 Fc-NKG2D Y152T/Y199F eNKG2D8 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSTHWMGLVHIPTNGSWQWEDGSILSPNLLTIIEMQKGDCALYASSFKGFIENCSTPNTYICMQRTV

SEQ ID NO: 52 - protein, 376 aa, synthetic construct
  REGION 1..376: note = Synthetic peptide human IgG1 Fc-NKG2D Y152V/Y199F eNKG2D9 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSVHWMGLVHIPTNGSWQWEDGSILSPNLLTIIEMQKGDCALYASSFKGFIENCSTPNTYICMQRTV

SEQ ID NO: 53 - protein, 376 aa, synthetic construct
  REGION 1..376: note = Synthetic peptide human IgG1 Fc-NKG2D Y199D eNKG2D10 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGDIENCSTPNTYICMQRTV

SEQ ID NO: 54 - protein, 376 aa, synthetic construct
  REGION 1..376: note = Synthetic peptide human IgG1 Fc-NKG2D Y199E eNKG2D11 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGEIENCSTPNTYICMQRTV

SEQ ID NO: 55 - protein, 376 aa, synthetic construct
  REGION 1..376: note = Synthetic peptide human IgG1 Fc-NKG2D Y152D/Y199D eNKG2D12 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSDHWMGLVHIPTNGSWQWEDGSILSPNLLTIIEMQKGDCALYASSFKGDIENCSTPNTYICMQRTV

SEQ ID NO: 56 - protein, 376 aa, synthetic construct
  REGION 1..376: note = Synthetic peptide human IgG1 Fc-NKG2D Y152E/Y199E eNKG2D13 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSEHWMGLVHIPTNGSWQWEDGSILSPNLLTIIEMQKGDCALYASSFKGEIENCSTPNTYICMQRTV

SEQ ID NO: 57 - protein, 376 aa, synthetic construct
  REGION 1..376: note = Synthetic peptide human IgG1 Fc-NKG2D Y152L eNKG2D14 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSLHWMGLVHIPTNGSWQWEDGSILSPNLLTIIEMQKGDCALYASSFKGYIENCSTPNTYICMQRTV

SEQ ID NO: 58 - protein, 376 aa, synthetic construct
  REGION 1..376: note = Synthetic peptide human IgG1 Fc-NKG2D Y152F/Y199F eNKG2D15 fusion
MDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSFHWMGLVHIPTNGSWQWEDGSILSPNLLTIIEMQKGDCALYASSFKGFIENCSTPNTYICMQRTV

SEQ ID NO: 59 - DNA, 1128 bp, synthetic construct
  misc_feature 1..1128: note = Synthetic polynucleotide, encoding human IgG1 Fc-NKG2D fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 60 - DNA, 1128 bp, synthetic construct
  misc_feature 1..1128: note = Synthetic polynucleotide, encoding human IgG1 Fc-NKG2D Y152A fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 61 - DNA, 1128 bp, synthetic construct
  misc_feature 1..1128: note = Synthetic polynucleotide, encoding human IgG1 Fc-NKG2D Y199A ectodomain
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcgctatagaaaactgttcaactccaaatacatacatctgcatgcaaaggactgtg

SEQ ID NO: 62 - DNA, 1128 bp, synthetic construct
  misc_feature 1..1128: note = Synthetic polynucleotide, encoding human IgG1 Fc-NKG2D Y152A/Y199A ectodomain
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggcgctatagaaaactgttcaactccaaatacatacatctgcatgcaaaggactgtg

SEQ ID NO: 63 - DNA, 1128 bp, synthetic construct
  misc_feature 1..1128: note = Synthetic polynucleotide, encoding human IgG1 Fc-NKG2D Y199F eNKG2D1 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 64 - DNA, 1128 bp, synthetic construct; identical to SEQ ID NO: 63
  misc_feature 1..1128: note = Synthetic polynucleotide, encoding human IgG1 Fc-NKG2D Y152S eNKG2D2 fusion
  source 1..1128: mol_type = other DNA

SEQ ID NO: 65 - DNA, 1128 bp, synthetic construct
  misc_feature 1..1128: note = Synthetic polynucleotide, encoding human IgG1 Fc-NKG2D Y152T eNKG2D3 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaactcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 66 - DNA, 1128 bp, synthetic construct
  misc_feature 1..1128: note = Synthetic polynucleotide, encoding human IgG1 Fc-NKG2D Y152V eNKG2D4 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagtgcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 67 - DNA, 1128 bp, synthetic construct
  misc_feature 1..1128: note = Synthetic polynucleotide, encoding human IgG1 Fc-NKG2D Y152A/Y199F eNKG2D5 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagctcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 68 - DNA, 1128 bp, synthetic construct
  misc_feature 1..1128: note = Synthetic polynucleotide, encoding human IgG1 Fc-NKG2D Y152L/Y199F eNKG2D6 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcactgcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 69 - DNA, 1128 bp, synthetic construct
  misc_feature 1..1128: note = Synthetic polynucleotide, encoding human IgG1 Fc-NKG2D Y152S/Y199F eNKG2D7 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaagtcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 70 - DNA, 1128 bp, synthetic construct
  misc_feature 1..1128: note = Synthetic polynucleotide, encoding human IgG1 Fc-NKG2D Y152T/Y199F eNKG2D8 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcaactcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 71 - DNA, 1128 bp, synthetic construct
  misc_feature 1..1128: note = Synthetic polynucleotide, encoding human IgG1 Fc-NKG2D Y152V/Y199F eNKG2D9 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagtgcattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 72 - DNA, 1128 bp, synthetic construct
  misc_feature 1..1128: note = Synthetic polynucleotide, encoding human IgG1 Fc-NKG2D Y199D eNKG2D10 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggcgatatagaaaactgttcaactccaaatacatacatctgcatgcaaaggactgtg

SEQ ID NO: 73 - DNA, 1128 bp, synthetic construct
  misc_feature 1..1128: note = Synthetic polynucleotide, encoding human IgG1 Fc-NKG2D Y199E eNKG2D11 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcataccattggatgggactagtacacattccaacaaatggatcttggcagtgggaagatggctccattctctcacccaacctactaacaataattgaaatgcagaagggagactgtgcactctatgcctcgagctttaaaggcgagatagaaaactgttcaactccaaatacatacatctgcatgcaaaggactgtg

SEQ ID NO: 74 - DNA, 1128 bp, synthetic construct
  misc_feature 1..1128: note = Synthetic polynucleotide, encoding human IgG1 Fc-NKG2D Y152D/Y199D eNKG2D12 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagatcattggatgggactagtacacattccaacaaatggatcttggcagtgggaagatggctccattctctcacccaacctactaacaataattgaaatgcagaagggagactgtgcactctatgcctcgagctttaaaggcgatatagaaaactgttcaactccaaatacatacatctgcatgcaaaggactgtg

SEQ ID NO: 75 - DNA, 1128 bp, synthetic construct
  misc_feature 1..1128: note = Synthetic polynucleotide, encoding human IgG1 Fc-NKG2D Y152E/Y199E eNKG2D13 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcagagcattggatgggactagtacacattccaacaaatggatcttggcagtgggaagatggctccattctctcacccaacctactaacaataattgaaatgcagaagggagactgtgcactctatgcctcgagctttaaaggcgagatagaaaactgttcaactccaaatacatacatctgcatgcaaaggactgtg

SEQ ID NO: 76 - DNA, 1128 bp, synthetic construct
  misc_feature 1..1128: note = Synthetic polynucleotide, encoding human IgG1 Fc-NKG2D Y152L eNKG2D14 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcactgcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 77 - DNA, 1128 bp, synthetic construct
  misc_feature 1..1128: note = Synthetic polynucleotide, encoding human IgG1 Fc-NKG2D Y152F/Y199F eNKG2D15 fusion
  source 1..1128: mol_type = other DNA
atggacccgaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaagatcgaaggacgcttcttaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcattccattggatgggactagtacacattccaacaaatggatcttggcagtgggaagatggctccattctctcacccaacctactaacaataattgaaatgcagaagggagactgtgcactctatgcctcgagctttaaaggcttcatagaaaactgttcaactccaaatacatacatctgcatgcaaaggactgtg

SEQ ID NO: 78 - protein, 181 aa, synthetic construct
  REGION 1..181: note = Synthetic peptide MICwed alpha1-alpha2
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETKEWTMPQSSRAQTLAMNVRNFLKEDAMETDTHYHAMHADCLQELRRYLKSGVVLRRT

SEQ ID NO: 79 - DNA, 543 bp, synthetic construct
  misc_feature 1..543: note = Synthetic polynucleotide, encoding MICwed alpha1-alpha2
  source 1..543: mol_type = other DNA
gagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccaaggaatggactatgcctcagagctctcgggcacagactctcgctatgaacgttagaaacttccttaaggaggatgctatggagaccgatactcactaccacgccatgcacgccgactgcctccaggaactgcggagatatctgaagtccggcgtggttttgagaagaacc

SEQ ID NO: 80 - protein, 181 aa, synthetic construct
  REGION 1..181: note = Synthetic peptide MIC25 alpha1-alpha2
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLSELRRYLKSGVVLRRT

SEQ ID NO: 81 - DNA, 543 bp, synthetic construct
  misc_feature 1..543: note = Synthetic polynucleotide, encoding MIC25 alpha1-alpha2
  source 1..543: mol_type = other DNA
gagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccctcgaatggactatgcctcagagctctcgggcacagactctcgctatgaacgttagaaacttccttaaggaggatgctatggagaccgatactcactaccacgccatgcgcgccgactgcctctctgaactgcggagatatctgaagtccggcgtggttttgagaagaacc

SEQ ID NO: 82 - protein, 330 aa, synthetic construct
  REGION 1..330: note = Synthetic peptide human IgG1 heavy chain CH1-CH2-CH3 D265A/N297A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 990 bp, synthetic construct
  misc_feature 1..990: note = human IgG1 heavy chain CH1-CH2-CH3 D265A/N297A
  source 1..990: mol_type = other DNA
gcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 84 - protein, 107 aa, synthetic construct [sequence omitted]

SEQ ID NO: 85 - DNA, 321 bp, synthetic construct
  misc_feature 1..321: note = Synthetic polynucleotide, encoding human kappa light chain
  source 1..321: mol_type = other DNA
agaaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgc

SEQ ID NO: 86 - protein, 120 aa, synthetic construct [sequence omitted]

SEQ ID NO: 87 - DNA, 360 bp, synthetic construct
  misc_feature 1..360: note = Synthetic polynucleotide, encoding trastuzumab VH
  source 1..360: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcg

SEQ ID NO: 88 - protein, 107 aa, synthetic construct [sequence omitted]

SEQ ID NO: 89 - DNA, 321 bp, synthetic construct
  misc_feature 1..321: note = Synthetic polynucleotide, encoding trastuzumab VL
  source 1..321: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaag

SEQ ID NO: 90 - protein, 121 aa, synthetic construct [sequence omitted]

SEQ ID NO: 91 - DNA, 363 bp, synthetic construct
  misc_feature 1..363: note = Synthetic polynucleotide, encoding rituximab VH
  source 1..363: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgca

SEQ ID NO: 92 - protein, 106 aa, synthetic construct [sequence omitted]

SEQ ID NO: 93 - DNA, 318 bp, synthetic construct
  misc_feature 1..318: note = Synthetic polynucleotide, encoding rituximab VL
  source 1..318: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaag

SEQ ID NO: 94 - protein, 450 aa, synthetic construct
  REGION 1..450: note = Synthetic peptide trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1350 bp, synthetic construct
  misc_feature 1..1350: note = Synthetic polynucleotide, encoding trastuzumab heavy chain
  source 1..1350: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 96 - protein, 213 aa, synthetic construct
  REGION 1..213: note = Synthetic peptide trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - DNA, 642 bp, synthetic construct
  misc_feature 1..642: note = Synthetic polynucleotide, encoding trastuzumab light chain
  source 1..642: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaagaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgctag

SEQ ID NO: 98 - protein, 451 aa, synthetic construct
  REGION 1..451: note = Synthetic peptide rituximab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 1353 bp, synthetic construct
  misc_feature 1..1353: note = Synthetic polynucleotide, encoding rituximab heavy chain
  source 1..1353: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgcagcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggatgtgtcacacgaggacccggaggtcaagttcaattggtacgtggacggcgtggaagtgcataacgcaaagaccaagcctcgggaggaacagtacaactcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 100 - protein, 212 aa, synthetic construct
  REGION 1..212: note = Synthetic peptide rituximab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - DNA, 639 bp, synthetic construct
  misc_feature 1..639: note = Synthetic polynucleotide, encoding rituximab light chain
  source 1..639: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgctag

SEQ ID NO: 102 - protein, 634 aa, synthetic construct
  REGION 1..634: note = Synthetic peptide trastuzumab HC_MICwed
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETKEWTMPQSSRAQTLAMNVRNFLKEDAMETDTHYHAMHADCLQELRRYLKSGVVLRRT

SEQ ID NO: 103 - DNA, 1902 bp, synthetic construct
  misc_feature 1..1902: note = Synthetic polynucleotide, encoding trastuzumab HC_MICwed
  source 1..1902: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccaaggaatggactatgcctcagagctctcgggcacagactctcgctatgaacgttagaaacttccttaaggaggatgctatggagaccgatactcactaccacgccatgcacgccgactgcctccaggaactgcggagatatctgaagtccggcgtggttttgagaagaacc

SEQ ID NO: 104 - protein, 634 aa, synthetic construct
  REGION 1..634: note = Synthetic peptide trastuzumab HC_MIC25
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLSELRRYLKSGVVLRRT

SEQ ID NO: 105 - DNA, 1902 bp, synthetic construct
  misc_feature 1..1902: note = Synthetic polynucleotide, encoding trastuzumab HC_MIC25
  source 1..1902: mol_type = other DNA
gaagtccaattggtcgaatcaggcggtggactcgtgcaacctggaggttcgttacgcttatcatgtgctgcaagtggatttaatattaaagatacctacatccactgggtacgtcaagctcccggcaagggtctcgagtgggtcgcacgcatttaccccaccaacggatacacgcgctacgccgattcagtgaagggacgtttcacaatctctgctgatactagcaaaaataccgcatacctccagatgaactctcttagggccgaggacacagctgtgtactactgtagccgttggggaggagacggtttttacgcaatggattactggggccaaggaaccctggtcacagtttcatcggcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagcctcacagcctccggtataatttgactgtactctcttgggatggctccgtgcagtccggctttctgactgaagttcatctcgacggtcaacctttcctgcgctgcgaccgacaaaaatgccgcgccaagccccaagggcagtgggccgaagatgtactgggaaacaagacctgggaccgggagacacgagacctgacaggctggggcaaggacttgcgcatgacactcgcccatatcaaggaccagaaggaaggattgcactctttgcaagagattcgcgtgtgtgaaatccacgaggacaattcaacgaggagctcccagcacttctattacgatggagaactcttcttgtcacagaacttggaaaccctcgaatggactatgcctcagagctctcgggcacagactctcgctatgaacgttagaaacttccttaaggaggatgctatggagaccgatactcactaccacgccatgcgcgccgactgcctctctgaactgcggagatatctgaagtccggcgtggttttgagaagaacc

SEQ ID NO: 106 - protein, 404 aa, synthetic construct
  REGION 1..404: note = Synthetic peptide rituximab LC_ULBP2.wt
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPS

SEQ ID NO: 107 - DNA, 1212 bp, synthetic construct
  misc_feature 1..1212: note = Synthetic polynucleotide, encoding rituximab LC_ULBP2.wt
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgcgcgacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 108 - protein, 187 aa, synthetic construct
  REGION 1..187: note = Synthetic peptide ULPB2 alpha1-alpha2 variant R80W
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPPMV

SEQ ID NO: 109 - DNA, 540 bp, synthetic construct
  misc_feature 1..540: note = Synthetic polynucleotide, encoding ULPB2 alpha1-alpha2 variant R80W
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 110 - DNA, 513 bp, synthetic construct
  misc_feature 1..513: note = Synthetic polynucleotide, encoding ULPB2 alpha1-alpha2 variant R80W NNK library
  misc_feature 457..458: note = n is a, c, g, or t
  misc_feature 460..461: note = n is a, c, g, or t
  misc_feature 463..464: note = n is a, c, g, or t
  misc_feature 466..467: note = n is a, c, g, or t
  misc_feature 469..470: note = n is a, c, g, or t
  source 1..513: mol_type = other DNA
gagccccatagcctcagctatgacattacggtgattcccaaatttcgcccaggaccacgttggtgcgccgtccagggtcaggtagatgaaaagactttcctgcattatgattgcggcaataaaaccgtgacgccggtatcgccgttaggcaaaaaattgaatgtcacgacagcgtggaaagcacagaacccggtgttgcgcgaggtagtcgatattttgacggaacaactctgggacattcagctcgagaattacaccccaaaagaaccgctgacgctgcaagcgcgtatgtcgtgcgaacaaaaagcagaaggtcactctagcgggagttggcagttttccttcgatgggcagatttttctgctgtttgattcggagaaacgcatgtggactacagtccacccgggtgcccggaaaatgaaagagaagtgggagaatgataaagtggtggccactnnknnknnknnknnktccatgggcgattgcattggctggttagaggattttctcatg

SEQ ID NO: 111 - protein, 180 aa, synthetic construct
  REGION 1..180: note = Synthetic peptide, ULBP2 alpha1-alpha2 variant ULBP2.C
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATILWQTSMGDCIGWLEDFLMGMDSTLEPS

SEQ ID NO: 112 - DNA, 540 bp, synthetic construct
  misc_feature 1..540: note = Synthetic polynucleotide, encoding ULBP2 alpha1-alpha2 variant ULBP2.C
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactattctgtggcagacttcgatgggagactgcatcggttggctggaagatttcctcatgggtatggactccactttggagccatcg

SEQ ID NO: 113 - protein, 180 aa, synthetic construct
  REGION 1..180: note = Synthetic peptide, ULBP2 alpha1-alpha2 variant ULBP2.R
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 114 - DNA, 540 bp, synthetic construct
  misc_feature 1..540: note = Synthetic polynucleotide, encoding ULBP2 alpha1-alpha2 variant ULBP2.R
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 115 - protein, 180 aa, synthetic construct
  REGION 1..180: note = Synthetic peptide, ULBP2 alpha1-alpha2 variant ULBP2.AA
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATMFWSWSMGDCIGWLEDFLMGMDSTLEPS

SEQ ID NO: 116 - DNA, 540 bp, synthetic construct
  misc_feature 1..540: note = Synthetic polynucleotide, encoding ULBP2 alpha1-alpha2 variant ULBP2.AA
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactatgttttggagttggtcgatgggagactgcatcggttggctggaagatttcctcatgggtatggactccactttggagccatcg

SEQ ID NO: 117 - protein, 180 aa, synthetic construct
  REGION 1..180: note = Synthetic peptide, ULBP2 alpha1-alpha2 variant ULBP2.AB
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMWQWSMGDCIGWLEDFLMGMDSTLEPS

SEQ ID NO: 118 - DNA, 540 bp, synthetic construct
  misc_feature 1..540: note = Synthetic polynucleotide, encoding ULBP2 alpha1-alpha2 variant ULBP2.AB
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactcttatgtggcagtggtcgatgggagactgcatcggttggctggaagatttcctcatgggtatggactccactttggagccatcg

SEQ ID NO: 119 - protein, 404 aa, synthetic construct
  REGION 1..404: note = Synthetic peptide rituximab LC_ULBP2.C
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATILWQTSMGDCIGWLEDFLMGMDSTLEPS

SEQ ID NO: 120 - DNA, 1212 bp, synthetic construct
  misc_feature 1..1212: note = Synthetic polynucleotide, encoding rituximab LC_ULBP2.C
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactattctgtggcagacttcgatgggagactgcatcggttggctggaagatttcctcatgggtatggactccactttggagccatcg

SEQ ID NO: 121 - protein, 404 aa, synthetic construct
  REGION 1..404: note = Synthetic peptide rituximab LC_ULBP2.R
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 122 - DNA, 1212 bp, synthetic construct
  misc_feature 1..1212: note = Synthetic polynucleotide, encoding rituximab LC_ULBP2.R
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 123 - protein, 404 aa, synthetic construct
  REGION 1..404: note = Synthetic peptide rituximab LC_ULBP2.AA
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATMFWSWSMGDCIGWLEDFLMGMDSTLEPS

SEQ ID NO: 124 - DNA, 1212 bp, synthetic construct
  misc_feature 1..1212: note = Synthetic polynucleotide, encoding rituximab LC_ULBP2.AA
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactatgttttggagttggtcgatgggagactgcatcggttggctggaagatttcctcatgggtatggactccactttggagccatcg

SEQ ID NO: 125 - protein, 404 aa, synthetic construct
  REGION 1..404: note = Synthetic peptide rituximab LC_ULBP2.AB
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMWQWSMGDCIGWLEDFLMGMDSTLEPS

SEQ ID NO: 126 - DNA, 1212 bp, synthetic construct
  misc_feature 1..1212: note = Synthetic polynucleotide, encoding rituximab LC_ULBP2.AB
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactcttatgtggcagtggtcgatgggagactgcatcggttggctggaagatttcctcatgggtatggactccactttggagccatcg

SEQ ID NO: 127 - protein, 180 aa, synthetic construct
  REGION 1..180: note = Synthetic peptide, ULBP2 alpha1-alpha2 variant ULBP2.S3
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 128 - DNA, 540 bp, synthetic construct
  misc_feature 1..540: note = Synthetic polynucleotide, encoding ULBP2 alpha1-alpha2 variant ULBP2.S3
  source 1..540: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcg

SEQ ID NO: 129 - protein, 404 aa, synthetic construct
  REGION 1..404: note = Synthetic peptide rituximab LC_ULBP2.S3
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 130 - DNA, 1212 bp, synthetic construct
  misc_feature 1..1212: note = Synthetic polynucleotide, encoding rituximab LC_ULBP2.S3
  source 1..1212: mol_type = other DNA
cagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcg

SEQ ID NO: 131 - protein, 634 aa, synthetic construct
  REGION 1..634: note = Synthetic peptide rituximab HC_ULBP2.R80W
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPS

SEQ ID NO: 132 - DNA, 1902 bp, synthetic construct
  misc_feature 1..1902: note = Synthetic polynucleotide, encoding rituximab HC_ULBP2.R80W
  source 1..1902: mol_type = other DNA
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgcagcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcg

SEQ ID NO: 133 - protein, 405 aa, synthetic construct
  REGION 1..405: note = Synthetic peptide trastuzumab LC_ULBP2.R
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 134 - DNA, 1215 bp, synthetic construct
  misc_feature 1..1215: note = Synthetic polynucleotide, encoding trastuzumab LC_ULBP2.R
  source 1..1215: mol_type = other DNA
gatatccaaatgactcaatcaccatcttcactctccgcgagcgtgggtgatcgggtcaccatcacatgtagggcgagccaagatgtgaataccgccgtcgcgtggtatcaacaaaagccgggaaaagcaccaaaactgcttatatactctgcatccttcctgtactctggggtgccaagccggttctccggtagtagatctggtactgactttacactcactatcagcagtctgcaacctgaggactttgcgacatactattgccagcagcactacacaaccccacctacatttggtcaggggacaaaggtggagatcaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactttgttgtgggggtggtcgatgggagactgcatcggttggctggaagatttcctcatgggtatggactccactttggagccatcg

SEQ ID NO: 135 - protein, 156 aa, synthetic construct
  REGION 1..156: note = Synthetic peptide NKG2D.wt ectodomain
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 136 - DNA, 468 bp, synthetic construct
  misc_feature 1..468: note = Synthetic polynucleotide, encoding NKG2D.wt ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagctaccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtg

SEQ ID NO: 137 - protein, 156 aa, synthetic construct
  REGION 1..156: note = Synthetic peptide NKG2D.YA ectodomain
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 138 - DNA, 468 bp, synthetic construct
  misc_feature 1..468: note = Synthetic polynucleotide, encoding NKG2D.YA ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtg

SEQ ID NO: 139 - protein, 156 aa, synthetic construct
  REGION 1..156: note = Synthetic peptide NKG2D.AF ectodomain
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTV

SEQ ID NO: 140 - DNA, 468 bp, synthetic construct
  misc_feature 1..468: note = Synthetic polynucleotide, encoding NKG2D.AF ectodomain
  source 1..468: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggcttcatcgagaactgcagcacccctaacacctacatctgtatgcagcggaccgtg

SEQ ID NO: 141 - protein, 69 aa, synthetic construct
  REGION 1..69: note = Synthetic peptide CD8alpha hinge and transmembrane domain
TTTPAPRPPTPAPTIASQPLSLRPEACRPAAGGAVHTRGLDFACDIYIWAPLAGTCGVLLLSLVITLYC

SEQ ID NO: 142 - DNA, 207 bp, synthetic construct
  misc_feature 1..207: note = Synthetic polynucleotide, encoding CD8alpha hinge and transmembrane domain
  source 1..207: mol_type = other DNA
accaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgc

SEQ ID NO: 143 - protein, 42 aa, synthetic construct
  REGION 1..42: note = Synthetic peptide 4-1BB
KRGRKKLLYIFKQPFMRPVQTTQEEDGCSCRFPEEEEGGCEL

SEQ ID NO: 144 - DNA, 126 bp, synthetic construct
  misc_feature 1..126: note = Synthetic polynucleotide, encoding 4-1BB
  source 1..126: mol_type = other DNA
aagcggggcagaaagaagctgctgtacatctttaagcagcccttcatgcggcccgtgcagaccacacaagaggaagatggctgctcctgcagattccccgaggaagaagaaggcggctgcgagctg

SEQ ID NO: 145 - protein, 112 aa, synthetic construct
  REGION 1..112: note = Synthetic peptide CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - DNA, 336 bp, synthetic construct
  misc_feature 1..336: note = Synthetic polynucleotide, encoding CD3zeta
  source 1..336: mol_type = other DNA
agagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 147 - protein, 239 aa, synthetic construct
  REGION 1..239: note = Synthetic peptide EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 148 - DNA, 717 bp, synthetic construct
  misc_feature 1..717: note = Synthetic polynucleotide, encoding EGFP
  source 1..717: mol_type = other DNA
atggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 149 - protein, 473 aa, synthetic construct
  REGION 1..473: note = Synthetic peptide CD8hingeTM_4-1BB_CD3zeta_EGFP
TTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 150 - DNA, 1419 bp, synthetic construct
  misc_feature 1..1419: note = Synthetic polynucleotide, encoding CD8hingeTM_4-1BB_CD3zeta_EGFP
  source 1..1419: mol_type = other DNA
accaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 151 - protein, 629 aa, synthetic construct
  REGION 1..629: note = Synthetic peptide NKG2D.wt_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 152 - DNA, 1887 bp, synthetic construct
  misc_feature 1..1887: note = Synthetic polynucleotide, encoding NKG2D.wt_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagctaccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 153 - protein, 629 aa, synthetic construct
  REGION 1..629: note = Synthetic peptide NKG2D.YA_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 154 - DNA, 1887 bp, synthetic construct
  misc_feature 1..1887: note = Synthetic polynucleotide, encoding NKG2D.YA_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 155 - protein, 629 aa, synthetic construct
  REGION 1..629: note = Synthetic peptide NKG2D.AF_CD8hingeTM_4-1BB_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGFIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 156 - DNA, 1887 bp, synthetic construct
  misc_feature 1..1887: note = Synthetic polynucleotide, encoding NKG2D.AF_CD8hingeTM_4-1BB_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggcttcatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 157 - protein, 473 aa, synthetic construct
  REGION 1..473: note = Synthetic peptide NKG2D.YA_CD8hingeTM_EGFP silent chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 158 - DNA, 1419 bp, synthetic construct
  misc_feature 1..1419: note = Synthetic polynucleotide, encoding NKG2D.YA_CD8hingeTM_EGFP silent chimeric antigen receptor
  source 1..1419: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgctcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 159 - protein, 225 aa, synthetic construct
  REGION 1..225: note = Synthetic peptide NKG2D.YA_CD8hingeTM silent chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYC

SEQ ID NO: 160 - DNA, 675 bp, synthetic construct
  misc_feature 1..675: note = Synthetic polynucleotide, encoding NKG2D.YA_CD8hingeTM silent chimeric antigen receptor
  source 1..675: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgc

SEQ ID NO: 161 - protein, 629 aa, synthetic construct
  REGION 1..629: note = Synthetic peptide NKG2D.YA_CD8hingeTM_4-1BBtraf2_CD3zeta_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQAAAGCSCRFPEAAAGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 162 - DNA, 1887 bp, synthetic construct
  misc_feature 1..1887: note = Synthetic polynucleotide, encoding NKG2D.YA_CD8hingeTM_4-1BBtraf2_CD3zeta_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagccgctgcaggctgctcctgcagattccccgaggctgccgcaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 163 - protein, 629 aa, synthetic construct
  REGION 1..629: note = Synthetic peptide NKG2D.YA_CD8hingeTM_4-1BB_CD3itam_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 164 - DNA, 1887 bp, synthetic construct
  misc_feature 1..1887: note = Synthetic polynucleotide, encoding NKG2D.YA_CD8hingeTM_4-1BB_CD3itam_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgttcaacgagctgaacctggggagaagagaagagttcgacgtgctggacaagcggagaggcagagatcctgagatgggcggcaagcccagacggaagaatcctcaagagggcctgtttaatgagctgcagaaagacaagatggccgaggccttcagcgagatcggaatgaagggcgagcgcagaagaggcaagggacacgatggactgttccagggcctgagcaccgccaccaaggatacctttgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 165 - protein, 629 aa, synthetic construct
  REGION 1..629: note = Synthetic peptide NKG2D.YA_CD8hingeTM_4-1BBtraf2_CD3itam_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQAAAGCSCRFPEAAAGGCELRVKFSRSADAPAYKQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 166 - DNA, 1887 bp, synthetic construct
  misc_feature 1..1887: note = Synthetic polynucleotide, encoding NKG2D.YA_CD8hingeTM_4-1BBtraf2_CD3itam_EGFP complete chimeric antigen receptor
  source 1..1887: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagccgctgcaggctgctcctgcagattccccgaggctgccgcaggcggctgcgagctgagagtgaagttcagccgttctgccgacgctcccgcctataagcagggacagaaccagctgttcaacgagctgaacctggggagaagagaagagttcgacgtgctggacaagcggagaggcagagatcctgagatgggcggcaagcccagacggaagaatcctcaagagggcctgtttaatgagctgcagaaagacaagatggccgaggccttcagcgagatcggaatgaagggcgagcgcagaagaggcaagggacacgatggactgttccagggcctgagcaccgccaccaaggatacctttgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 167 - protein, 517 aa, synthetic construct
  REGION 1..517: note = Synthetic peptide NKG2D.YA_CD8hingeTM_4-1BB_EGFP complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 168 - DNA, 1551 bp, synthetic construct
  misc_feature 1..1551: note = Synthetic polynucleotide, encoding NKG2D.YA_CD8hingeTM_4-1BB_EGFP complete chimeric antigen receptor
  source 1..1551: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgtcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 169 - protein, 269 aa, synthetic construct
  REGION 1..269: note = Synthetic peptide NKG2D.YA_CD8hingeTM_4-1BB complete chimeric antigen receptor
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCEL

SEQ ID NO: 170 - DNA, 807 bp, synthetic construct
  misc_feature 1..807: note = Synthetic polynucleotide, encoding NKG2D.YA_CD8hingeTM_4-1BB complete chimeric antigen receptor
  source 1..807: mol_type = other DNA
atggcattgcctgttacagctctgctgctgcccctggctctgcttctgcatgctgccagacctctgttcaatcaagaggtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctg

SEQ ID NO: 171 - protein, 736 aa, synthetic construct
  REGION 1..736: note = Synthetic peptide CD19scFv_CD8hingeTM_4-1BB_CD3zeta_EGFP complete chimeric antigen receptor
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 172 - DNA, 2208 bp, synthetic construct
  misc_feature 1..2208: note = Synthetic polynucleotide, encoding CD19scFv_CD8hingeTM_4-1BB_CD3zeta_EGFP complete chimeric antigen receptor
  source 1..2208: mol_type = other DNA
atggcattgcctgttacagctctgctgctgcccctggctctgcttctgcatgctgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaa

SEQ ID NO: 173 - protein, 1115 aa, synthetic construct
  REGION 1..1115: note = Synthetic peptide CD19scFv_CD8hingeTM_4-1BB_CD3zeta _EGFP-T2A-NKG2D.YA_CD8hingeTM
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYKLEGGGEGRGSLLTCGDVEENPGPRMLLLVTSLLLCELPHPAFLLIPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYC

SEQ ID NO: 174 - DNA, 3345 bp, synthetic construct
  misc_feature 1..3345: note = Synthetic polynucleotide, encoding CD19scFv_CD8hing eTM_4-1BB_CD3zeta_EGFP-T2A-NKG2D.YA_CD8hingeTM
  source 1..3345: mol_type = other DNA
atggcattgcctgttacagctctgctgctgcccctggctctgcttctgcatgctgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctaccaccacaccagctcctagacctccaactcctgctcctacaatcgccagccagcctctgtctctgaggccagaagcttgtagacctgctgcaggcggagccgtgcatacaagaggactggatttcgcctgcgacatctacatctgggcccctctggctggaacatgtggcgtgctgctgctgagcctggtcatcaccctgtactgcagcctgaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagccgttctgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatcaggctctggttctggcagcggcagcatggtgtctaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagtccgccatgcctgagggctacgtgcaagagcggaccatcttctttaaggacgacggcaactacaagaccagggccgaagtgaagtttgagggcgacaccctggtcaaccggatcgagctgaagggcatcgacttcaaagaggatggcaacatcctgggccacaagctcgagtacaactacaacagccacaacgtgtacatcatggccgacaagcagaagaacggcatcaaggccaacttcaagatccggcacaacatcgaggacggcagcgttcagctggccgatcactaccagcagaacacccctatcggagatggccctgtgctgctccccgacaatcactacctgagcacacagagcgccctgagcaaggaccccaacgagaagagggatcacatggtgctgctggaatttgtgaccgccgcaggcatcaccctcggcatggacgaactgtacaaactcgagggcggcggagagggcagaggaagtcttctaacatgcggtgacgtggaggagaatcccggccctaggatgcttctcctggtgacaagccttctgctctgtgagttaccacacccagcattcctcctgatcccactgttcaaccaggaggttcagattccgctgaccgaaagctactgtggaccatgtcccaagaactggatctgctacaaaaacaattgttatcaatttttcgacgaaagcaagaattggtacgaaagccaggcttcatgcatgagccaaaatgcatccctgctgaaggtgtacagcaaggaggaccaggacctgctgaaacttgtcaaatccgctcactggatgggactggtccatattcctacaaatggatcctggcagtgggaggatggctctatcctgtctcccaatctcctgactatcatagagatgcagaagggagattgtgcattgtatgctagttcattcaagggatacattgaaaactgctcaacacctaatacctacatatgtatgcagcgcacagtgcagatccctctgaccgagagctactgtggcccctgtcctaagaactggatctgctacaagaacaactgctaccagttcttcgacgagagcaagaattggtacgagagccaggccagctgcatgagccagaatgccagcctgctgaaggtgtacagcaaagaggaccaggatctgctgaagctggtcaagagcgcccactggatgggactcgtgcacatccctacaaacggcagctggcagtgggaggacggctctatcctgtctcctaacctgctgaccatcatcgagatgcagaagggcgactgcgccctgtacgccagcagctttaagggctacatcgagaactgcagcacccctaacacctacatctgtatgcagcggaccgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 175 - protein, 161 aa, synthetic construct
  REGION 1..161: note = Synthetic peptide FLAG_6xHis_mutIL2
GSSGSSDYKDDDDKHHHHHHHHGSSGSSAPTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 176 - DNA, 483 bp, synthetic construct
  misc_feature 1..483: note = Synthetic polynucleotide, encoding FLAG_6xHis_mutIL2
  source 1..483: mol_type = other DNA
ggaagtagcggtagtagtgattacaaggacgatgacgacaagcaccaccatcatcatcatcaccacggtagcagcggcagcagtgcccccacctctagcagcacaaagaagacccagctgcaactggaacacctcctgctggacctgcagatgatcctgaacggcatcaacaactacaagaaccccaagctgaccgccatgctgaccaaaaagttttacatgcccaagaaggccaccgagcttaaacacctgcaatgccttgaggaggagctgaagcccctggaggaggtactgaacctggcccagagcaagaactttcatctgaggcccagggacctgattagcaacatcaacgtgatcgtgttggagttgaagggcagcgagaccacgttcatgtgcgagtacgccgacgagacggccaccatagtggagtttcttaacaggtggatcaccttctcacagtctatcatcagcaccctgacc

SEQ ID NO: 177 - protein, 343 aa, synthetic construct
  REGION 1..343: note = Synthetic peptide ULBP2.R80W_FLAG_6xHis_mutIL2
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSGTGSSGSSDYKDDDDKHHHHHHHHGSSGSSAPTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 178 - DNA, 1029 bp, synthetic construct
  misc_feature 1..1029: note = Synthetic polynucleotide, encoding ULBP2.R80W_FLAG_6xHis_mutIL2
  source 1..1029: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcgggtaccggtagtagcggtagtagtgattacaaggacgatgacgacaagcaccaccatcatcatcatcaccacggtagcagcggcagcagtgcccccacctctagcagcacaaagaagacccagctgcaactggaacacctcctgctggacctgcagatgatcctgaacggcatcaacaactacaagaaccccaagctgaccgccatgctgaccaaaaagttttacatgcccaagaaggccaccgagcttaaacacctgcaatgccttgaggaggagctgaagcccctggaggaggtactgaacctggcccagagcaagaactttcatctgaggcccagggacctgattagcaacatcaacgtgatcgtgttggagttgaagggcagcgagaccacgttcatgtgcgagtacgccgacgagacggccaccatagtggagtttcttaacaggtggatcaccttctcacagtctatcatcagcaccctgacc

SEQ ID NO: 179 - protein, 343 aa, synthetic construct
  REGION 1..343: note = Synthetic peptide ULBP2.S2_FLAG_6xHis_mutIL2
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMRIWSMGDCIGWLEDFLMGMDSTLEPSGTGSSGSSDYKDDDDKHHHHHHHHGSSGSSAPTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 180 - DNA, 1029 bp, synthetic construct
  misc_feature 1..1029: note = Synthetic polynucleotide, encoding ULBP2.S2_FLAG_6xHis_mutIL2
  source 1..1029: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactctgatgaggatttggtcgatgggagactgcatcggttggctggaagatttcctcatgggtatggactccactttggagccatcgggtaccggtagtagcggtagtagtgattacaaggacgatgacgacaagcaccaccatcatcatcatcaccacggtagcagcggcagcagtgcccccacctctagcagcacaaagaagacccagctgcaactggaacacctcctgctggacctgcagatgatcctgaacggcatcaacaactacaagaaccccaagctgaccgccatgctgaccaaaaagttttacatgcccaagaaggccaccgagcttaaacacctgcaatgccttgaggaggagctgaagcccctggaggaggtactgaacctggcccagagcaagaactttcatctgaggcccagggacctgattagcaacatcaacgtgatcgtgttggagttgaagggcagcgagaccacgttcatgtgcgagtacgccgacgagacggccaccatagtggagtttcttaacaggtggatcaccttctcacagtctatcatcagcaccctgacc

SEQ ID NO: 181 - protein, 412 aa, synthetic construct
  REGION 1..412: note = Synthetic peptide ULBP2.R80W_Fc1
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 182 - DNA, 1236 bp, synthetic construct
  misc_feature 1..1236: note = Synthetic polynucleotide, encoding ULBP2.R80W_Fc1
  source 1..1236: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgatgtcattccactatttctcgatgggagactgcatcggttggctggaagatttcctcatgggtatggactccactttggagccatcggagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactatgacactaccccgcccgtgctggattccgacggaagcttcttcctgtactccgacctgaccgtggacaagtcgagatggcagcagggaaatgtgttcagctgctccgtgatgcatgaggcgctgcacaaccactacacccagaagtcactgagcctctcccccggaaag

SEQ ID NO: 183 - protein, 368 aa, synthetic construct
  REGION 1..368: note = Synthetic peptide Fc2_mutIL2
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSAPTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 184 - DNA, 1104 bp, synthetic construct
  misc_feature 1..1104: note = Synthetic polynucleotide, encoding Fc2_mutIL2
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgcccccacctctagcagcacaaagaagacccagctgcaactggaacacctcctgctggacctgcagatgatcctgaacggcatcaacaactacaagaaccccaagctgaccgccatgctgaccaaaaagttttacatgcccaagaaggccaccgagcttaaacacctgcaatgccttgaggaggagctgaagcccctggaggaggtactgaacctggcccagagcaagaactttcatctgaggcccagggacctgattagcaacatcaacgtgatcgtgttggagttgaagggcagcgagaccacgttcatgtgcgagtacgccgacgagacggccaccatagtggagtttcttaacaggtggatcaccttctcacagtctatcatcagcaccctgacc

SEQ ID NO: 185 - protein, 349 aa, synthetic construct
  REGION 1..349: note = Synthetic peptide Fc2_mutIL15
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 186 - DNA, 1047 bp, synthetic construct
  misc_feature 1..1047: note = Synthetic polynucleotide, encoding Fc2_mutIL15
  source 1..1047: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcaattgggtgaacgtgatttctgacttgaagaaaatcgaggacttgattcaaagtatgcacatagacgcaacgctctatactgagagtgatgttcatccctcttgtaaagttaccgctatgaagtgtttcttgctcgaactccaagttatcagtctggagagcggtgatgcctccatacacgatacagtcgaaaaccttatcattctcgcaaataactcattgagtagcaatggcaatgttacagagtctgggtgtaaggaatgcgaagaacttgaagagaaaaacataaaggagttcctccaatcattcgtgcatattgtccagatgtttatcaacactagc

SEQ ID NO: 187 - protein, 412 aa, synthetic construct
  REGION 1..412: note = Synthetic peptide ULBP2.S2_Fc1
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMRIWSMGDCIGWLEDFLMGMDSTLEPSEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 188 - DNA, 1236 bp, synthetic construct
  misc_feature 1..1236: note = Synthetic polynucleotide, encoding ULBP2.S2_Fc1
  source 1..1236: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactctgatgaggatttggtcgatgggagactgcatcggttggctggaagatttcctcatgggtatggactccactttggagccatcggagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactatgacactaccccgcccgtgctggattccgacggaagcttcttcctgtactccgacctgaccgtggacaagtcgagatggcagcagggaaatgtgttcagctgctccgtgatgcatgaggcgctgcacaaccactacacccagaagtcactgagcctctcccccggaaag

SEQ ID NO: 189 - protein, 412 aa, synthetic construct
  REGION 1..412: note = Synthetic peptide ULBP2.S3_Fc1
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPSEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 190 - DNA, 1236 bp, synthetic construct
  misc_feature 1..1236: note = Synthetic polynucleotide, encoding ULBP2.S3_Fc1
  source 1..1236: mol_type = other DNA
gagccccatagtctgagctacgacatcacagttattcccaagttcaggcccggaccgcgctggtgtgccgtgcaaggacaagtcgacgaaaaaacctttcttcattacgattgcggaaataagactgtaacgccagtctctcctttaggtaagaagttaaacgtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcggagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactatgacactaccccgcccgtgctggattccgacggaagcttcttcctgtactccgacctgaccgtggacaagtcgagatggcagcagggaaatgtgttcagctgctccgtgatgcatgaggcgctgcacaaccactacacccagaagtcactgagcctctcccccggaaag

SEQ ID NO: 191 - protein, 368 aa, synthetic construct
  REGION 1..368: note = Synthetic peptide Fc2_wtIL2 (wild-type)
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 192 - DNA, 1104 bp, synthetic construct
  misc_feature 1..1104: note = Synthetic polynucleotide, encoding Fc2_wtIL2
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagcgctccaacctctagctctacgaagaaaactcaactccagcttgagcatttgcttttggaccttcaaatgatattgaacgggattaacaattacaagaaccccaaactgacgcgaatgcttacattcaaattttacatgccaaagaaggccaccgagctgaaacacttgcaatgtcttgaggaagagctgaagccattggaggaggtcctgaatttggctcaaagtaagaacttccacctgcggccaagggatctcataagcaacataaatgtcattgtattggaactgaaagggagcgaaaccacctttatgtgcgagtatgcagatgaaactgcaaccatagtcgagttcctcaatcgctggataactttcgcgcagtcaattatatcaacactcacc

SEQ ID NO: 193 - protein, 368 aa, synthetic construct
  REGION 1..368: note = Synthetic peptide Fc2_IL21.wt (wild-type)
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSQGQDRHMIRMRQLIDIVDQLKNYVNDLVPEFLPAPEDVETNCEWSAFSCFQKAQLKSANTGNNERIINVLIKKLKRKPPSTNAGRRQKHRLTCPSCDSYEKKPPKEFLERFKSLLQKMIHQHLSSRTHGSEDS

SEQ ID NO: 194 - DNA, 1104 bp, synthetic construct
  misc_feature 1..1104: note = Synthetic polynucleotide, encoding Fc2_IL21.wt (wild-type)
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagccagggtcaagaccgccatatgatccgaatgcgacagctcattgatattgtcgatcaattgaaaaattacgtgaatgatcttgtaccggagttcctccccgcaccggaggacgttgaaacgaattgtgagtggtcagcattttcttgctttcagaaggctcaactcaagagtgcaaacacgggtaacaacgagcgcattatcaatgttctcatcaaaaagctgaaacgaaaaccgcctagcaccaacgcaggcagacgacagaagcaccggctcacgtgcccaagttgcgattcttatgagaaaaagccaccgaaagaattcctggagcggttcaagtccctcttgcagaaaatgattcatcagcatctctccagcaggacacacggctccgaggactcc

SEQ ID NO: 195 - protein, 368 aa, synthetic construct
  REGION 1..368: note = Synthetic peptide Fc2_IL21.D18A
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSQGQDRHMIRMRQLIDIVAQLKNYVNDLVPEFLPAPEDVETNCEWSAFSCFQKAQLKSANTGNNERIINVLIKKLKRKPPSTNAGRRQKHRLTCPSCDSYEKKPPKEFLERFKSLLQKMIHQHLSSRTHGSEDS

SEQ ID NO: 196 - DNA, 1104 bp, synthetic construct
  misc_feature 1..1104: note = Synthetic polynucleotide, encoding Fc2_IL21.D18A
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagccagggtcaagaccgccatatgatccgaatgcgacagctcattgatattgtcgcacaattgaaaaattacgtgaatgatcttgtaccggagttcctccccgcaccggaggacgttgaaacgaattgtgagtggtcagcattttcttgctttcagaaggctcaactcaagagtgcaaacacgggtaacaacgagcgcattatcaatgttctcatcaaaaagctgaaacgaaaaccgcctagcaccaacgcaggcagacgacagaagcaccggctcacgtgcccaagttgcgattcttatgagaaaaagccaccgaaagaattcctggagcggttcaagtccctcttgcagaaaatgattcatcagcatctctccagcaggacacacggctccgaggactcc

SEQ ID NO: 197 - protein, 368 aa, synthetic construct
  REGION 1..368: note = Synthetic peptide Fc2_IL21.E109R
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGGGGSQGQDRHMIRMRQLIDIVDQLKNYVNDLVPEFLPAPEDVETNCEWSAFSCFQKAQLKSANTGNNERIINVLIKKLKRKPPSTNAGRRQKHRLTCPSCDSYEKKPPKEFLRRFKSLLQKMIHQHLSSRTHGSEDS

SEQ ID NO: 198 - DNA, 1104 bp, synthetic construct
  misc_feature 1..1104: note = Synthetic polynucleotide, encoding Fc2_IL21.E109R
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagccagggtcaagaccgccatatgatccgaatgcgacagctcattgatattgtcgatcaattgaaaaattacgtgaatgatcttgtaccggagttcctccccgcaccggaggacgttgaaacgaattgtgagtggtcagcattttcttgctttcagaaggctcaactcaagagtgcaaacacgggtaacaacgagcgcattatcaatgttctcatcaaaaagctgaaacgaaaaccgcctagcaccaacgcaggcagacgacagaagcaccggctcacgtgcccaagttgcgattcttatgagaaaaagccaccgaaagaattcctgcggcggttcaagtccctcttgcagaaaatgattcatcagcatctctccagcaggacacacggctccgaggactcc

SEQ ID NO: 199 - protein, 368 aa, synthetic construct
  REGION 1..368: note = Synthetic peptide Fc2_IL21.D18A.E109R
EPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSQGQDRHMIRMRQLIDIVAQLKNYVNDLVPEFLPAPEDVETNCEWSAFSCFQKAQLKSANTGNNERIINVLIKKLKRKPPSTNAGRRQKHRLTCPSCDSYEKKPPKEFLRRFKSLLQKMIHQHLSSRTHGSEDS

SEQ ID NO: 200 - DNA, 1104 bp, synthetic construct
  misc_feature 1..1104: note = Synthetic polynucleotide, encoding Fc2_IL21.D18A.E109R
  source 1..1104: mol_type = other DNA
gagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaggaggtggcagccagggtcaagaccgccatatgatccgaatgcgacagctcattgatattgtcgcacaattgaaaaattacgtgaatgatcttgtaccggagttcctccccgcaccggaggacgttgaaacgaattgtgagtggtcagcattttcttgctttcagaaggctcaactcaagagtgcaaacacgggtaacaacgagcgcattatcaatgttctcatcaaaaagctgaaacgaaaaccgcctagcaccaacgcaggcagacgacagaagcaccggctcacgtgcccaagttgcgattcttatgagaaaaagccaccgaaagaattcctgcggcggttcaagtccctcttgcagaaaatgattcatcagcatctctccagcaggacacacggctccgaggactcc

SEQ ID NO: 201 - protein, 233 aa, synthetic construct
  REGION 1..233: note = Synthetic peptide Fc2
MDPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 202 - DNA, 699 bp, synthetic construct
  misc_feature 1..699: note = Synthetic polynucleotide, encoding Fc2
  source 1..699: mol_type = other DNA
atggaccccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccggaaggagctgaccaaaaaccaagtgtccctgacttgccttgtgaaggggttctacccttctgacattgccgtcgaatgggaatcgaacggacagcctgaaaacaactataagactaccccgcccgtgctgaagtccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag